(19) United States
(12) Patent Application Publication
(10) Pub. No.: US 2025/0177301 A1
(43) Pub. Date: Jun. 5, 2025

(54) TRIPLEX NANOPARTICLES

(71) Applicant: UNM Rainforest Innovations, Albuquerque, NM (US)

(72) Inventors: Achraf Noureddine, Albuquerque, NM (US); Charles Jeffrey Brinker, Albuquerque, NM (US); Bindica Poudel, Charlottesville, VA (US); Lien Tang, Albuquerque, NM (US)

(21) Appl. No.: 18/834,513

(22) PCT Filed: Jan. 31, 2023
(86) PCT No.: PCT/US2023/061675

§ 371 Date: Jul. 30, 2024

Related U.S. Application Data
(60) Provisional application No. 63/304,962, filed on Jan. 31, 2022.

(51) Int. Cl. A61K 9/1271; A61K 31/4745; A61K 31/475; A61K 31/52; A61K 31/573; A61K 31/675; A61K 31/7068

(52) U.S. Cl. CPC A61K 9/1271; A61K 31/4745; A61K 31/475; A61K 31/52; A61K 31/573; A61K 31/675; A61K 31/7068

(57) ABSTRACT

A multi-layer (multi-lamellar) coated nanoparticle is provided wherein the nanoparticle comprises a first coat comprising a positively charged polymer or a positively charged lipd bilayer and a second coat comprising an anionic bilayer and optionally one or more distinct cargo molecules, one or more distinct targeting moieties, or any combination thereof, as well as methods of making and using the nanoparticle.

### CROSS-REFERENCE TO RELATED APPLICATIONS

This application claims the benefit of the filing date of U.S. application No. 63/304,962, filed on Jan. 31, 2022, the disclosure of which is incorporated by reference herein.

### INCORPORATION BY REFERENCE OF SEQUENCE LISTING

A Sequence Listing is provided herewith as an xml file, “2305487.xml” created on Jan. 30, 2023 and having a size of 28,071 bytes. The content of the xml file is incorporated by reference herein in its entirety.

### SUMMARY

The present disclosure provides for nanoparticles, e.g., mesoporous silica nanoparticles (MSNs), surrounded by two layers (coats), one of which is positively charged and the other of which is negatively charged. In one embodiment, one of the layers comprises lipids, e.g., a lipid bilayer, thereby forming a lipid coated nanoparticle. In one embodiment, the nanoparticles comprise a cargo, e.g., a therapeutic, prophylactive or diagnostic agent, and/or one or more targeting moieties. In one embodiment, a first cargo may be between the core particle, e.g., MSN, and the first (inner) layer of the nanoparticle, which optionally comprises a targeting molecule in the negatively charged (outer) layer. In one embodiment, a first cargo may be within the core particle, e.g., MSN. In one embodiment, a first cargo may be embedded in the first layer of the nanoparticle. In one embodiment, a first cargo may be between the first layer of the nanoparticle and the negatively charged layer which optionally comprises a targeting molecule. In one embodiment, the nanoparticles are monodisperse and are stable in vivo.

In one embodiment, a population of lipid coated nanoparticles comprising a population of mesoporous silica nanoparticles is provided, each of said nanoparticles comprising at least one lipid layer, e.g., a bilayer, and another layer which is in one embodiment not a lipid layer so as to form a multi-lamellar coating. In one embodiment, a population of monosized lipid coated nanoparticles comprising a population of monosized mesoporous silica nanoparticles (mMSNs) is provided, each of said nanoparticles comprising a lipid layer, e.g., a bilayer, and another layer which is in one embodiment not a lipid layer so as to form a multi-lamellar coating (e.g., fused thereto), e.g., completely covering the surface of the MSNs. In one embodiment said population of lipid coated nanoparticles exhibits a polydispersity index (Pdl) of less than about 0.1 to no more than about 0.2. In certain embodiments, the population of lipid coated nanoparticles exhibits a polydispersity index of less than about 0.1.

In one embodiment, a population of lipid coated nanoparticles is provided comprising a population of MSNs to each of which is coated with a positively charged layer, which optionally is not a positively charged lipid layer, and an anionic lipid bilayer, said positively charged layer covering the surface of the nanoparticle (a “coating”) and said lipid bilayer covering the surface of said coated MSNs. In one embodiment, at least one lipid in the bilayer is at a weight ratio of at least about 200% by weight, e.g., about 200% to about 1000% by weight (e.g., about 2:1 to about 10:1) of said population of nanoparticles, wherein said lipid is at least one anionic lipid, e.g., at least one zwitterionic lipid, optionally comprising cholesterol and further optionally comprising a lipid containing a functional group to which may be covalently or non-covalently bonded a targeting or other functional moiety.

MSNs may range in diameter from about 1 nm to about 500 nm, about 2 nm to about 10 nm, about 5 nm to about 350 nm, about 10 nm to about 300 nm, about 15 nm to about 250 nm, about 20 nm to about 200 nm, about 25 nm to about 350 nm, or about 20 nm to about 100 nm. In one embodiment, the pores of the MSNs are about 1 to about 10 nm, about 5 nm to about 10 nm or about 6 nm to about 14 nm in diameter. In one embodiment, the MSNs are about 80 to about 100 nm in diameter. In one embodiment, the lipid nanoparticles are less than about 400 nm in diameter. In one embodiment, the lipid nanoparticles are greater than about 150 nm in diameter. In one embodiment, the lipid nanoparticles are about 200 nm to about 350 nm in diameter. In one embodiment, in a population of monodisperse MSNs, each MSN does not vary more than about 5% from the average diameter of the MSNs in the population and exhibits a polydispersity index (Pdl) of less than about 0.1, or less than about 0.2, e.g., less than about 0.1.

Lipid coated nanoparticles exhibit colloidal and/or storage stability. In particular, monosized lipid coated nanoparticles exhibit colloidal stability and storage stability in aqueous solution (water, buffer, blood, plasma, etc.) such that the lipid coated nanoparticles maintain their monodispersity for a period of at least several hours (about 2, 3, 4, 5 or 6 hours), at least about 12 hours, at least about 24 hours, at least about two days, three days, four days, five days, six days, one week, two weeks, four weeks, two months, three months, four months, five months, six months, one year or longer. In one embodiment, the lipid coated nanoparticles are stored in phosphate buffered saline solutions, saline solution (isotonic saline solution), other aqueous buffer solutions, or water (especially distilled water). The monosized lipid coated nanoparticles maintain their monodispersity in blood, plasma, serum and/or other body fluids for extended periods of time.

Lipid coated nanoparticles may further comprise at least one additional component, for example, a cell targeting moiety (species), e.g., a peptide, antibody, such as a monoclonal antibody, an affibody or a small molecule moiety which binds to a cell, among others); a fusogenic peptide that promotes endosomal escape of lipid coated nanoparticles; a cargo, including one or more drugs (e.g., an anti-cancer agent, anti-viral agent, antibiotic, antifungal agent, etc.); a polynucleotide, such as encapsulated DNA, double stranded linear DNA, a plasmid DNA, small interfering RNA, small hairpin RNA, microRNA, a peptide, polypeptide or protein, an imaging agent, or a mixture thereof, among others), wherein one of said cargo components is optionally conjugated further with a nuclear localization sequence.

In certain embodiments, lipid coated nanoparticles comprise a nanoporous silica core; a positively charged layer; a negatively charged lipid bilayer; and a cargo comprising at least one therapeutic agent (for example, an anti-viral agent, antibiotic or an anti-cancer agent which optionally facilitates cancer cell death, such as a traditional small molecule, a macromolecular cargo, e.g., mRNA, antisense oligonucletoides (ASOs), siRNA such as S565, S7824 and/or S10234, among others, shRNA or a protein toxin such as a ricin toxin A-chain or diphtheria toxin A-chain) and/or a packaged plasmid DNA (in certain embodiments-histone packaged) disposed within the nanoporous silica core (e.g., supercoiled as otherwise described herein in order to more efficiently package the DNA into lipid coated nanoparticles as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Lipid coated nanoparticles may include a targeting peptide which targets cells for therapy (e.g., cancer cells in tissue to be treated, infected cells or other cells requiring therapy) such that binding of the lipid coated nanoparticle to the targeted cells is specific and enhanced and a fusogenic peptide that promotes endosomal escape of lipid coated nanoparticles and encapsulated DNA. Lipid coated nanoparticles may be used in therapy or diagnostics, more specifically to treat cancer and other diseases, including viral infections, including hepatocellular (liver) and other cancers which occur secondary to viral infection. In other aspects, lipid coated nanoparticles use binding peptides which selectively bind to cancer tissue (MET peptides for example, as disclosed in WO 2012/149376, and CRLF2 peptides, for example, as disclosed in WO 2013/103614, relevant portions of which applications are incorporated by reference herein).

In another embodiment, a storage stable composition is provided comprising a population of monosized lipid coated nanoparticles in an aqueous solution such as buffered saline, water, or isotonic saline solutions, among others.

In an additional embodiment, pharmaceutical compositions (e.g., storage stable compositions) are provided comprising an effective amount of a population of lipid coated nanoparticles as described herein, in combination with at least one carrier, additive and/or excipient.

In still another embodiment, a method of producing lipid coated nanoparticles is provided. The method includes providing a population of MSNs and exposing said nanoparticles to, for example, a polyamine, and a population of lipids or monosized liposomes comprising at least one lipid (the lipid mixture may be simple or complex, depending on the ultimate function of the lipid coated nanoparticle), the liposome to MSN mass ratio being at least, for example, 1:1 or 2:1 (the liposomes may have an internal surface area which is greater than the external surface area of the nanoparticles), wherein the nanoparticles are exposed to, for example, the polyamine and liposomes in an aqueous solution (e.g., an aqueous buffer solution such as phosphate buffered saline solution, although other solutions, including buffered saline solutions may be used). In one embodiment, the lipid coated nanoparticles have a hydrodynamic diameter of greater than about 100 nm and low PDI value of less than about 0.2, or less than 0.1. In one embodiment, the liposomes, e.g., monosized liposomes, poiyamine and MSNs are combined in buffered saline solution, sonicated or otherwise agitated for several seconds up to a minute or more) to allow the polyamine to coat the MSNs and the liposomal lipid to coat/fuse to the polyamine and the non-coated liposomes in solution may be removed/separated from the lipid coated nanoparticles, for instance, by centrifugation. The pelleted lipid coated nanoparticles are redispersed at least once (e.g., in phosphate buffered saline solution or other solutions in which the lipid coated nanoparticles are to be stored and/or used) via agitation (e.g., sonication).

In still another embodiment, therapeutic methods comprise administering a pharmaceutical composition comprising a population of lipid coated nanoparticles to a patient in need in order to treat a disease state or condition from which the patient is suffering. The disease state includes but is not limited to cancer, a viral infection, a bacterial infection, a fungal infection or other infection.

Thus, the disclosure provides therapeutic formulations with increased therapeutic efficacy in vivo. The dramatic therapeutic efficacy of numerous targeted nanoparticle-based delivery platforms observed in vitro has rarely translated into similar performance in vivo. In exceedingly complex living systems, particle polydispersity, sequestration, and instability have limited the delivery of cargos to specific cell types despite the presence of effective targeting agents. Described herein is a process for the synthesis and characterization of monodisperse mesoporous silica-supported lipid bilayer nanoparticles (e.g., lipid coated nanoparticles) designed to exhibit in vivo stability and targeted cell binding. Specific aspects of the modular synthesis protocol allow for precise control of size, shape, pore structure, and surface chemistry that can be tailored to achieve colloidal stability and targeted binding for a range of applications. The demonstrated in vitro stability attributed to the supported lipid bilayer may be confirmed in vivo using real-time, high resolution microscopic analysis in a chicken embryo chorioallantoic membrane (CAM) model combined with hydrodynamic size analysis. Moreover, by establishing synthetic protocols that enabled colloidal stability and avoided non-specific binding of non-targeted lipid coated nanoparticles, antibody conjugation may be demonstrated to direct highly selective binding in vivo.

In another embodiment, a multilamellar lipid coated nanoparticle T cell vaccine is provided that delivers full length viral protein and/or plasmid encoded viral protein to antigen presenting cells (APCs). The multilamellar lipid coated nanoparticle contains a nanoparticle core and at least an inner polyamine layer and an outer lipid bilayer and, optionally, an inner aqueous layer which separates the core from the inner layer and further optionally, an outer aqueous layer which separates the inner layer from the outer lipid bilayer. The outer lipid bilayer of the lipid coated nanoparticle is functionalized with a Toll-like receptor (TLR) agonist (e.g., monophosphoryl lipid A (MPLA) and/or flagellin) to facilitate and initiate an immunological signaling cascade, said outer bilayer further including a fusogenic peptide such as octa-arginine (R8) peptide to induce cellular uptake of the lipid coated nanoparticle. In addition, full length viral proteins may be distributed throughout the outer lipid bilayer or said optional inner aqueous layer or outer aqueous layer, e.g., the outer aqueous layer, to be processed in the endosome and presented to CD4+ T cells through the MHC Class II pathway. The inner polyamine layer is functionalized with an endosomolytic peptide such as H5WYG (or alternatively, INF7, GALA, KALA, or RALA) which enhances endosomal escape. In some embodiments, the lipid coated nanoparticle includes an internal porous silica core loaded with plasmid DNA encoding viral proteins and/or viral proteins fused to ubiquitin to be processed in the cytoplasm and presented to CD8+ T cells through the MHC Class I pathway. The plasmid is transcribed into a template and further translated into viral proteins, which are labeled with ubiquitin, a regulatory protein that tags and directs proteins to the proteasome for further degradation in preparation for antigen presentation.

In one embodiment, a multilamellar lipid coated nanoparticle is provided comprising a nanoporous silica or metal oxide core and a multilamellar polyamine coating and a lipid bilayer coating, said core comprising an inner, for example, polyamine layer and an outer anionic lipid bilayer and optionally, an inner aqueous layer separating said core and said inner polyamine layer and an optional outer aqueous layer separating said inner layer and said outer lipid bilayer, said outer lipid bilayer of said multilamellar lipid bilayer comprising: at least targeting molecule, e.g., GRP78 or a TLR ligand such as MPLA and/or flaggellin to initiate an immunological signaling cascade; optionally a fusogenic peptide (e.g., octa-arginine (R8) peptide) to induce cellular uptake of the lipid coated nanoparticle; and optionally at least one cell targeting species which selectively binds to a target (peptide, receptor or other target) on APCs; said inner polyamine layer of said multilamellar bilayer comprising an endosomolytic peptide (e.g., H5WYG) to enhance endosomal escape, and said outer lipid bilayer and/or said inner layer and/or said optional outer aqueous layer and/or said optional inner aqueous layer optionally further comprising an antigen such as at least one viral antigen (e.g., a full length viral protein, which is optionally ubiquitylated as a fusion protein) distributed throughout said outer lipid bilayer, said inner layer and/or said optional outer aqueous layer and/or said optional inner aqueous layer; said nanoporous core of said lipid coated nanoparticle optionally being loaded with a cargo such as a pre-ubiquitylated viral protein (e.g., as a single peptide chain that includes ubiquitin or a ubiquitylated viral antigen) or a plasmid DNA encoding viral protein, which is optionally labeled with ubiquitin.

Multilamellar lipid coated nanoparticles may also comprise a drug (including, for example, a nucleic acid such as miRNA, siRNA, shRNA, RNAi, mRNA or a plasmid, an anti-microbial agent or chemotherapeutic) or other agent, e.g., to enhance an immunogenic response such as an adjuvant.

### BRIEF DESCRIPTION OF THE FIGURES

FIG. 1. Exemplary embodiments to make triplex nanoparticles.

FIGS. 2A-2F. Physico-chemical features of the siRNA loaded Triplex nanoplatform FIGS. 3A-3B. CryoTEM images show the halo indicative of the coating by soft shell (polymer/lipid) and the incorporation of protein/targeting agents.

FIG. 4. Cell viability assay using the mesoporous silica nanoparticle with different components. Ctrl sirna, PCA3, sh3 and sh4 are siRNA loaded within the MSN-PEI-lipid system. TAR: Targeting agent conjugated.

FIG. 5. Targeting agent (GRP78) that targets prostate cancer cell line is conjugated at different ratio to the nanoparticle surface. The more targeting agent we have, the quicker and more pronounced is the uptake. Lipid coated nanoparticle=lipid-coated nanoparticle.

FIG. 6. Delivery of GFP mRNA with triplex nanoparticles and other control (negative and lipofectamine). Data show that highest GFP expression occurred with a triplex using 12 nm pore size MSN. Cells are LNCAP prostate cancer cells. mRNA amount incubated follows the lipofectamine online procedure.

### DETAILED DESCRIPTION

These and/or other embodiments of may be readily gleaned from the following description.

### Definitions

Where a range of values is provided, it is understood that each intervening value, to the tenth of the unit of the lower limit unless the context clearly dictates otherwise, between the upper and lower limit of that range and any other stated or intervening value in that stated range is encompassed. The upper and lower limits of these smaller ranges may independently be included in the smaller ranges is also encompassed, subject to any specifically excluded limit in the stated range. Where the stated range includes one or both of the limits, ranges excluding either both of those included limits are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described.

It must be noted that as used herein and in the appended claims, the singular forms “a,” “and” and “the” include plural references unless the context clearly dictates otherwise.

Reference to “about” a value or parameter herein includes (and describes) variations that are directed to that value or parameter per se. For example, description referring to “about X” includes description of “X”.

The term “monodisperse” and “monosized” are used synonymously to describe both mesoporous particles, e.g., nanoparticles (although the particles may range up to about 6 microns in diameter) and lipid coated nanoparticles (i.e., mesoporous nanoparticles having a fused lipid bilayer on the surface of the nanoparticles) which are monodisperse.

The term “monosized mesoporous silica nanoparticles” or MSNs is used to describe a population of monosized (monodispersed) mesoporous silica nanoparticles. Example particles are produced using a solution-based surfactant directed self-assembly strategy conducted under basic conditions, followed by hydrothermal treatment to provide MSNs with tunable core structure, pore sizes and shape. Certain methods for producing silica nanoparticles are described in Lin et al., 2005; Lin et al., 2010; Lin et al., 2011; Chen et al., 2013; Bayu et al., 2009; Wang et al., 2012; Shen et al., 2014; Huang et al., 2011; and Yu et al., 2011, among others. MSNs may be provided in various shapes, including spherical, oval, hexagonal, dendritic, cylindrical, rod-shaped, disc-like, tubular and polyhedral pursuant to the above-described methods. Monodispersity can also be described as having a polydispersity index (Pdl or DPI) of about 0.1 to about 0.2, less than about 0.2, or less than about 0.1.

The synthetic procedures for providing monodisperse MSNs may be varied to vary the contents and size of the MSNs, as well as the pore size. In typical synthesis, MSNs are produced using a solution based surfactant directed self-assembly strategy conducted under basic conditions (e.g., triethylamine or other weak base), followed by a hydrothermal treatment. Size adjustment may be facilitated by increasing the concentration of catalyst (e.g., ammonium hydroxide). Increasing the concentration of the catalyst will increase the size of the resulting MSNs, whereas decreasing the concentration of the catalyst will decrease the size of the resulting MSNs. Increasing the amount of silica precursor (e.g., TEOS) will also increase the particle size, as will decreasing the temperature during synthesis. Decreasing the amount of silica precursor and/or increasing the temperature during synthesis will decrease the particle size. All of the above parameters may be modified to adjust the sizes of the mesopores within the nanoparticles. To change the nature of the silica particles, amine-containing silanes such as N-(2-aminoethyl)-3-aminopropyltrimethoxysilane (AEPTMS) or 3-aminopropyltriethoxysilane (APTES) may be added to the solution containing TEOS or other silica precursor. The addition of an amine-containing silane will produce a silica particle with a zeta potential (mV) with a less negative to neutral/positive zeta potential, depending on the amount of amine-containing silane including in the reaction mixture to form the nanoparticles. The nanoparticles have a zeta potential (mV) ranging from about −50 mV to about +35 mV depending upon the amount of amine containing silane added to the synthesis (e.g., from about 0.01% up to about 50% by weight, often about 0.1% to about 20% by weight, about 0.25% to about 15% by weight, about 0.5% to about 10% by weight), with a greater amount of amine containing silane increasing the zeta potential and a lesser amount (to none) providing a nanoparticle with a negative zeta potential.

Surfactants which can be used in the synthesis of MSNs include for example, octyltrimethylammonium bromide, decyltrimethylammonium bromide, dodecyltrimethylammonium bromide, tetradecyltrimethylammonium bromide, benzyldimethylhexadecylammonium chloride, hexadecyltrimethylammonium bromide, hexadecyltrimethylammonium chloride, octadecyltrimethylammonium bromide, octadecyltrimethylammonium chloride, dihexadecyldimethylammonium bromide, dimethyldioctadecylammonium bromide, dimethylditetradecylammonium bromide, didodecyldimethylammonium bromide, didecyldimethylammonium bromide and didecyldimethylammonium bromide, among others.

The term “lipid coated nanoparticle” is used to describe a porous nanoparticle surrounded by a lipid bilayer. In some embodiments, the porous nanoparticle is made of a material comprising silica, polystyrene, alumina, titania, zirconia, or generally metal oxides, organometallates, organosilicates or mixtures thereof.

The term “lipid” is used to describe the components which are used to form lipid bilayers on the surface of nanoparticles.

Porous nanoparticulates used in lipid coated nanoparticles include mesoporous silica nanoparticles and core-shell nanoparticles. The porous nanoparticulates can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycaprolactone (PCL), polyanhydrides, poly(ortho) esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

A porous spherical silica nanoparticle may be used for the lipid coated nanoparticles and is surrounded by a supported lipid or polymer bilayer or multi-layer. Various embodiments provide nanostructures and methods for constructing and using the nanostructures and providing lipid coated nanoparticles. Many of the lipid coated nanoparticles in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Texas, USA or from Discovery Scientific, Inc., Vancouver, British Columbia. Multimodal silica nanoparticles may be readily prepared using the procedure of Carroll, et al., Langmuir, 25, 13540-13544 (2009). Lipid coated nanoparticles can be readily obtained using methodologies known in the art. The examples section of the present application provides certain methodology for obtaining lipid coated nanoparticles. Lipid coated nanoparticles may be readily prepared, including lipid coated nanoparticles comprising lipids which are fused to the surface of the silica nanoparticle. See, for example, Liu et al., 2009; Liu et al., 2009: Liu et al., 2009; Lu et al., 1999, Lipid coated nanoparticles may be prepared according to the procedures which are presented in Ashley et al., 2011; Lu et al., 1999; Caroll et al., 2009, and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, a prism or a whisker. A nanoparticle may include particles having two or more of the aforementioned shapes. In one embodiment, a cross-sectional geometry of the particle may be one or more of circular, ellipsoidal, triangular, toroidal, rectangular or polygonal. In one embodiment, a nanoparticle may consist essentially of non-spherical particles, especially prisms. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. In one embodiment, a plurality of nanoparticles may consist essentially of hexagonal prism nanoparticles.

The term “monosized lipid coated nanoparticles” is used to describe a population of monosized (monodisperse) lipid coated nanoparticles comprising a lipid bilayer fused onto a MSNs as otherwise described herein. In some embodiments, monosized lipid coated nanoparticles are prepared by fusing the lipids in monosized unilamellar liposomes onto the MSNs in aqueous buffer (e.g., phosphate buffered solution) or other solution at about room temperature, although slightly higher and lower temperatures may be used. The unilamellar liposomes which are fused onto the MSNs are prepared by sonication and extrusion according to the method of Akbarzadeh et al., 2013 and are monodisperse with hydrodynamic diameters of less than about 100 nm, often about 65-95 nm, most often about 90-95 nm, although unilamellar liposomes which can be used may fall outside this range depending on the size of the MSNs to which lipids are to be fused and low PDI values (generally, less than about 0.5, e.g., less than 0.2). The mass ratio of liposomes to MSNs used to create monosized lipid coated nanoparticles which have a single lipid bilayer completely surrounding the MSNs is that amount sufficient to provide a liposome interior surface area which equals or exceeds the exterior surface area of the MSNs to which the lipid is to be fused. This often is provided in a mass ratio of liposomes to MSNs of at least about 2:1, often up to about 10:1 or more, with a range often used being about 2:1 to about 5:1. The resulting lipid coated nanoparticles are monosized (monodisperse). Monosized lipid coated nanoparticles may exhibit extended storage stability in aqueous solution, e.g., providing a SLB on the lipid coated nanoparticle which has a transition temperature Tm which is greater than the storage, experimental or administration/therapeutic conditions under which the lipid coated nanoparticles are stored and/or used. Often the lipid coated nanoparticle is at least about 25-30 nm in diameter larger than the diameter of the MSNs.

The phrase “effective average particle size” as used herein to describe a multiparticulate (e.g., a porous nanoparticulate) means that all particles therein are of an average diameter or within +5% of the average diameter. In certain embodiments, nanoparticulates have an effective average particle size (diameter) of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 35 nm, less than about 25 nm, as measured by light-scattering methods, microscopy, or other appropriate methods. In exemplary aspects, the average diameter of MSNs ranges from about 75 nm to about 150 nm, often about 75 to about 130 nm, often about 75 nm to about 100 nm.

The term “patient” or “subject” is used throughout the specification within context to describe an animal, generally a mammal, especially including a domesticated animal and for example a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment. Exemplary bioactive agents include the compounds which are used to treat cancer or a disease state or condition which occurs secondary to cancer and may include anti-viral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease state or condition, including improvement in the disease state or condition through lessening, inhibition, suppression or elimination of at least one symptom, delay in progression of the disease, prevention, delay in or inhibition of the likelihood of the onset of the disease state and/or condition, etc. In the case of microbial infections, these terms also apply to microbial (e.g., viral or bacterial) infections and may include, in certain particularly favorable embodiments the eradication or elimination (as provided by limits of diagnostics) of the microbe (e.g., a virus or a bacterium) which is the causative agent of the infection.

Treatment, as used herein, encompasses both prophylactic and therapeutic treatment, e.g., of cancer (including inhibiting metastasis or recurrence of a cancer in remission), but also of other disease states, including microbial infections such as bacterial, fungal, protest, aechaea, and viral infections, especially including HBV and/or HCV. Compounds can, for example, be administered prophylactically to a mammal in advance of the occurrence of disease to reduce the likelihood of that disease. Prophylactic administration, e.g., a vaccine, is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections. In another embodiment of therapeutic administration, administration of the present compounds is effective to decrease the likelihood of infection or re-infection by a microbe and/or to decrease the symptom(s) or severity of an infection.

The term “prophylactic administration” refers to any action in advance of the occurrence of disease to reduce the likelihood of that disease or any action to reduce the likelihood of the subsequent occurrence of disease in the subject. Compositions can, for example, be administered prophylactically to a mammal in advance of the occurrence of disease to enhance an immunogenic effect and/or reduce the likelihood of that disease, generally a viral disease. Prophylactic administration is effective to reduce or decrease the likelihood of the subsequent occurrence of disease in the mammal, or decrease the severity of disease (inhibition) that subsequently occurs, especially including a microbial (e.g., a viral or bacterial) infection and/or cancer, its metastasis or recurrence.

The term “antihepatocellular cancer agent” is used throughout the specification to describe an anti-cancer agent which may be used to Inhibit, treat or reduce the likelihood of hepatocellular cancer, or the metastasis of that cancer, especially secondary to a viral infection such as HBV and/or HCV. Anti-cancer agents which may find use Include for example nexavar (sorafenib), sunitinib, bevacizumab, tarceva (erlotinib), tykerb (lapatinib), and mixtures thereof. In addition, other anti-cancer agents may also be used, where such agents are found to inhibit metastasis of cancer, in particular, hepatocellular cancer.

The term “targeting active species” is used to describe a compound or moiety which is complexed or covalently bonded to the surface of a lipid coated nanoparticle which binds to a moiety on the surface of a cell to be targeted so that the lipid coated nanoparticle may selectively bind to the surface of the targeted cell and deposit its contents into the cell. In one embodiment, the targeting active species is a “targeting peptide” including a polypeptide including an antibody or antibody fragment, an aptamer, or a carbohydrate, among other species which bind to a targeted cell. A targeting active species may be peptide of a particular sequence which binds to a receptor or other polypeptide in cancer cells and allows the targeting of lipid coated nanoparticles to particular cells which express a peptide (be it a receptor or other functional polypeptide) to which the targeting peptide binds. Exemplary targeting peptides include, for example, SP94 free peptide (H2N-SFSIILTPILPL-COOH, SEQ ID NO: 3), SP94 peptide modified with a C-terminal cysteine for conjugation with a crosslinking agent (H2N-GLFHAIAHFIHGGWHGLIHGWYGGC-COOH (SEQ ID. NO: 4) or an 8 mer polyarginine (H₂N-RRRRRRRR-COOH, SEQ ID NO:5), a modified SP94 peptide (H₂N-SFSIILTPILPLEEEGGC-COOH, SEQ ID NO:6) or a MET binding peptide or CRLF2 binding peptide as disclosed in WO 2012/149376, published Nov. 1, 2012 and CRLF2 peptides, for example as disclosed in WO 2013/103614, published Jul. 11, 2013, relevant portions of which applications are incorporated by reference herein. Other targeting peptides are known in the art. Targeting peptides may be complexed or covalently linked to the lipid bilayer through use of a crosslinking agent as otherwise described herein.

The term “MET binding peptide” or “MET receptor binding peptide” is used to describe any peptide that binds the MET receptor. MET binding peptides include at least five (5) 7-mer peptides which have been shown to bind MET receptors on the surface of cancer cells with enhanced binding efficiency. Several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g., hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7 mer peptide sequences show substantial binding to MET receptor and may be useful as targeting peptides for use on lipid coated nanoparticles.

(SEQ ID NO: 7)
ASVHFPP (Ala-Ser-Val-His-Phe-Pro-Pro)
(SEQ ID NO: 8)
TATFWFQ (Thr-Ala-Thr-Phe-Trp-Phe-Gln)
(SEQ ID NO: 9)
TSPVALL (Thr-Ser-Pro-Val-Ala-Leu-Leu)
(SEQ ID NO: 10)
IPLKVHP (lle-Pro-Leu-Lys-Val-His-Pro)
(SEQ ID NO: 11)
WPRLTNM (Trp-Pro-Arg-Leu-Thr-Asn-Met)

Each of these peptides may be used alone or in combination with other MET peptides within the above group or with other targeting peptides which may assist in binding lipid coated nanoparticles n to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally crosslinked onto the lipid bilayer surface of the lipid coated nanoparticles. Fusogenic peptides are incorporated onto lipid coated nanoparticles in order to facilitate or assist escape from endosomal bodies and to facilitate the introduction of lipid coated nanoparticles into targeted cells to effect an intended result (therapeutic and/or diagnostic as otherwise described herein). Representative fusogenic peptides for use in lipid coated nanoparticles include but are not limited to H5WYG peptide, H₂N-GLFHAIAHFIHGGWHGLIHGWYGGC-COOH (SEQ ID. NO: 12) or an 8 mer polyarginine (H₂N-RRRRRRRR-COOH, SEQ ID NO:13), among others known in the art. Additional fusogenic peptides include RALA peptide (NH₂-WEARLARALARALARHLARALARALRAGEA-COOH, SEQ ID NO: 14), KALA peptide (NH₂-WEAKLAKALAKALAKHLAKALAKALKAGEA-COOH), SEQ ID. NO: 15), GALA (NH₂-WEAALAEALAEALAEHLAEALAEALEALAA-COOH, SEQ ID NO:16) and INF7 (NH₂-GLFEAIEGFIENGWEGMIDGWYG-COOH, SEQ ID. NO: 17), among others.

Thus, the terms “cell penetration peptide,” “fusogenic peptide” and “endosomolytic peptide” are used to describe a peptide which aids lipid coated nanoparticle translocation across a lipid bilayer, such as a cellular membrane or endosome lipid bilayer and is optionally crosslinked onto a lipid bilayer surface of the lipid coated nanoparticles. Endosomolytic peptides are a sub-species of fusogenic peptides as described herein. In both the multilamellar and single layer lipid coated nanoparticle embodiments, the non-endosomolytic fusogenic peptides (e.g., electrostatic cell penetrating peptide such as R8 octaarginine) are incorporated onto the lipid coated nanoparticles at the surface of the lipid coated nanoparticle in order to facilitate the introduction of lipid coated nanoparticles into targeted cells (APCs) to effect an intended result (to instill an immunogenic and/or therapeutic response as described herein). The endosomolytic peptides (often referred to in the art as a subset of fusogenic peptides) may be incorporated in the surface lipid bilayer of the lipid coated nanoparticle or in a lipid sublayer of the multilamellar lipid coated nanoparticle in order to facilitate or assist in the escape of the lipid coated nanoparticle from endosomal bodies. Representative electrostatic cell penetration (fusogenic) peptides for use in lipid coated nanoparticles include an 8 mer polyarginine (H₂N-RRRRRRRR-COOH, SEQ ID NO: 1), among others known in the art, which are included in lipid coated nanoparticles in order to enhance the penetration of the lipid coated nanoparticle into cells. Representative endosomolytic fusogenic peptides (“endosomolytic peptides) include H5WYG peptide, H₂N-GLFHAIAHFIHGGWHGLIHGWYGGC-COOH (SEQ ID. NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH), SEQ ID. NO: 19), GALA (NH₂-WEAALAEALAEALAEHLAEALAEALEALAA-COOH, SEQ ID NO: 20) and INF7 (NH₂-GLFEAIEGFIENGWEGMIDGWYG-COOH, SEQ ID. NO: 21), among others. At least one endosomolytic peptide is included in lipid coated nanoparticles in combination with a viral antigen (often pre-ubiquitinylated) and/or a viral plasmid (which expresses viral protein or antigen) in order to produce CD8+ cytotoxic T cells pursuant to a MHC class I pathway.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides and other functional moieties (for example toll receptor agonists for immunogenic) to the phospholipid bilayer, to link nuclear localization sequences to histone proteins for packaging supercoiled plasmid DNA and in certain instances, to crosslink lipids in the lipid bilayer of the lipid coated nanoparticles. There are a large number of crosslinking agents which may be used in many commercially available or available in the literature. Exemplary crosslinking agents for use, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[ß-Maleimidopropionic acid]hydrazide (BMPH), NHS-(PEG)ₙ-maleimide, succinimidyl-[(N-maleimidopropionamido)-tetracosaethyleneglycol]ester (SM (PEG)₂₄), and succinimidyl 6-[3′-(2-pyridyldithio)-propionamido]hexanoate (LC-SPDP), among others.

The term “antigen presenting cell” “APC” or “accessory cell” is a cell in the body that displays foreign antigens complexed with major histocompatibility complexes (MHCs) on their surfaces through antigen presentation. These cells include dendritic cells (DCs), macrophages, B-cells which express a B cell receptor (BCR) and specific antibody which binds to the BCR, certain activated epithelial cells (any cell which expresses MHC class II molecules) and any nucleated cell which expresses MHC class I molecules). T cells often recognize these complexes through T-cell receptors. APCs process antigens and present them to T-cells.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility desired. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the lipid coated nanoparticles. There are a large number of crosslinking agents which may be used, many commercially available or available in the literature. Exemplary crosslinking agents for use include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[β-Maleimidopropionamido]hexanoate (SMPH), N-[ß-Maleimidopropionic acid]hydrazide (BMPH), NHS-(PEG)ₙ-maleimide, succinimidyl-[(N-maleimidopropionamido)-tetracosaethyleneglycol]ester (SM (PEG)₂₄), and succinimidyl 6-[3′-(2-pyridyldithio)-propionamido]hexanoate (LC-SPDP), among others.

The term “targeting active species” is used to describe a compound or moiety which binds to a moiety on the surface of a targeted cell so that the lipid coated nanoparticle may selectively bind to the surface of the targeted cell and deposit its contents into the cell. The targeting active species for use may be a targeting peptide as otherwise described herein, a polypeptide including an antibody or antibody fragment, an aptamer, or a carbohydrate, among other species which bind to a targeted cell, especially an antigen presenting cell.

The term “toll-like receptor (TLR) agonist” or “TLR agonist” refers to a moiety on the surface of the lipid coated nanoparticles which are provided to bind to toll-like receptors on cells containing these receptors and initiate an immunological signaling cascade in providing an immunogenic response to lipid coated nanoparticles. These agonists enhance or otherwise favorably influence the engagement of T-cell subsets to both stimulate immune responses and make certain cells better targets for immune-mediated destruction TLR agonists which can be used in lipid coated nanoparticles include a number of compounds/compositions which have shown activity as agonists for toll-like receptors 1 through 9 (TLR 1, TLR 2, TLR 3, TLR 4, TLR 5, TLR 6, TLR 7, TLR 8 and TLR 9). These compounds/compositions include Pam3Cys, HMGB1, Porins, HSP, GLP (agonists for TLR1/2); BCG-CWS, HP-NAP, Zymosan, MALP2, PSK (agonists for TLR 2/6); dsRNA, Poly AU, Poly ICLC, Poly I: C (agonists for TLR 3); LPS, EDA, HSP, Fibrinogen, Monophosphoryl Lipid A (MPLA) (agonists for TLR 4); Flagellin (agonist for TLR 5); Imiquimod (agonist for TLR 7); and ssRNA, PolyG10 and CpG (agonists for TLR 8), as described by Kaczanowka et al., 2013. TLR agonists are covalently linked to components of the lipid bilayer using conventional chemistry as described herein above for the fusogenic peptides.

The term “ubiquitin” or “ubiquitinylation” is used throughout the present specification to refer to the use of a ubiquitin protein in combination with a viral antigen (e.g., a full length viral protein) as a fusion protein or conjugated via an isopeptide bond. Ubiquitylation of viral proteins generally speeds the development of immunogenicity. Ubiquitin, also referred to as ubiquitous immunopoietic polypeptide, is a protein involved in ubiquitination in the cell and, facilitates the immunogenic response raised after the lipid coated nanoparticles are introduced into antigen presenting cells (APCs) by facilitating/regulating the degradation of proteins (via the proteasome and lysosome), coordinating the cellular localization of proteins, activating and inactivating proteins and modulating protein-protein interactions, resulting in an enhancement in antigen processing in both professional and non-professional APCs through exogenous and endogenous pathways.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions, alone or in combination with another agent. It is noted that prophylaxis will rarely be 100% effective; consequently the terms prevention and reducing the likelihood are used to denote the fact that within a given population of patients or subjects, administration with compounds will reduce the likelihood or inhibit a particular condition or disease state (in particular, the worsening of a disease state such as the growth or metastasis of cancer) or other accepted indicators of disease progression from occurring.

“Amine-containing silanes” include, but are not limited to, a primary amine, a secondary amine or a tertiary amine functionalized with a silicon atom, and may be a monoamine or a polyamine such as diamine. For example, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used to modify the MSNs.

The term “reporter” is used to describe an imaging agent or moiety which is incorporated into the phospholipid bilayer or cargo of lipid coated nanoparticles according to an embodiment and provides a signal which can be measured. The moiety may provide a fluorescent signal or may be a radioisotope which allows radiation detection, among others. Exemplary fluorescent labels for use in lipid coated nanoparticles (e.g., via conjugation or adsorption to the lipid bilayer or silica core, although these labels may also be incorporated into cargo elements such as DNA, RNA, polypeptides and small molecules which are delivered to cells by the lipid coated nanoparticles, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-IT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of cancer (cancer tissue) in a patient and or the progress of therapy in a patient or subject.

The term “histone-packaged supercoiled plasmid DNA” is used to describe an exemplary component of lipid coated nanoparticles, which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the lipid coated nanoparticles). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into lipid coated nanoparticles (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments which include a MET binding peptide complexed to the lipid coated nanoparticle.

The terms “coadminister” and “coadministration” are used synonymously to describe the administration of at least one of the lipid coated nanoparticle compositions in combination with at least one other agent, often at least one additional anti-cancer agent (as otherwise described herein), which are specifically disclosed herein in amounts or at concentrations which would be considered to be effective amounts at or about the same time. While it is envisioned that coadministered compositions/agents be administered at the same time, agents may be administered at times such that effective concentrations of both (or more) compositions/agents appear in the patient at the same time for at least a brief period of time. Alternatively, in certain aspects, it may be possible to have each coadministered composition/agent exhibit its inhibitory effect at different times in the patient, with the ultimate result being the inhibition and treatment of cancer, especially including hepatocellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising lipid coated nanoparticles and optionally, to treat any type of cancer, in particular hepatocellular or cervical cancer, among numerous others. Anti-cancer compounds which can be formulated with compounds include, for example, Exemplary anti-cancer agents which may be used include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespen, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES (diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser (But) 6,Azglyl0](pyro-Glu-His-Trp-Ser-Tyr-D-Ser (But)-Leu-Arg-Pro-Azgly-NH₂ acetate [C59H84N18014-(C₂H₄O₂) x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erlotinib, lapatinib, canertinib, ABX-EGF antibody, erbitux, EKB-569, PKI-166, GW-572016, Ionafarnib, BMS-214662, tipifarnib, amifostine, NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anti-cancer agent which may be used to inhibit, treat or reduce the likelihood of hepatocellular cancer, or the metastasis of that cancer. Anti-cancer agents which may find use include for example, nexavar (sorafenib), sunitinib, bevacizumab, tarceva (erlotinib), tykerb (lapatinib) and mixtures thereof. In addition, other anti-cancer agents may also be used, where such agents are found to inhibit metastasis of cancer, in particular, hepatocellular cancer.

The term “anti (HCV)-viral agent” is used to describe a bioactive agent/drug which inhibits the growth and/or elaboration of a virus, including mutant strains such as drug resistant viral strains. Exemplary anti-viral agents include anti-HIV agents, anti-HBV agents and anti-HCV agents. In certain aspects, especially where the treatment of hepatocellular cancer is the object of therapy, the inclusion of an anti-hepatitis C agent or anti-hepatitis B agent may be combined with other traditional anti-cancer agents to effect therapy, given that hepatitis B virus (HBV) and/or hepatitis C virus (HCV) is often found as a primary or secondary infection or disease state associated with hepatocellular cancer. Anti-HBV agents which may be used, either as a cargo component in the lipid coated nanoparticle or as an additional bioactive agent in a pharmaceutical composition which includes a population of lipid coated nanoparticles includes such agents as Hepsera (adefovir dipivoxil), lamivudine, entecavir, telbivudine, tenofovir, emtricitabine, clevudine, vallorcitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in include such agents as boceprevir, daclatasvir, asunaprevir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use which can be included as cargo in lipid coated nanoparticles include, for example, including nucleoside reverse transcriptase inhibitors (NRTI), other non-nucleoside reverse transcriptase inhibitors (i.e., those which are not representative), protease inhibitors, fusion inhibitors, among others, exemplary compounds of which may include, for example, 3TC (Lamivudine), AZT (Zidovudine), (-)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof.

### Exemplary Monosized Nanostructures

In an embodiment, the nanostructures include a mesoporous silica core-shell structure which comprises a porous particle core surrounded by a shell of lipid such as a bilayer, but possibly a monolayer or multi-layer. The porous silica particle coreinclude, for example, a porous nanoparticle surrounded by a polyamine layer and a lipid bilayer. In some non-limiting instances, these lipid bilayer surrounded nanostructures are referred to as “lipid coated nanoparticles” or “functional lipid coated nanoparticles” and have a supported lipid bilayer membrane structure. However, the porous nanoparticle may be surrounded by other naturally occurring or synthetic polymers and those may also be referred to as “lipid coated nanoparticles.” In some embodiments, the porous particle core of the lipid coated nanoparticles can be loaded with various desired species (“cargo”), including small molecules (e.g., anti-cancer agents as otherwise described herein), large molecules (e.g., including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g., fluorescent green protein, among others) or semiconductor quantum dots or combinations thereof. In certain exemplary aspects, the lipid coated nanoparticles are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-cancer agents, anti-viral agents and antibiotics, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In some embodiments, the lipid bilayer of the lipid coated nanoparticles can provide biocompatibility and can be modified to possess targeting species including, for example, targeting peptides including antibodies, aptamers, and PEG (polyethylene glycol) to allow, for example, further stability of the lipid coated nanoparticles and/or a targeted delivery into a bioactive cell.

In some embodiments, the lipid coated nanoparticles particle size distribution is monodisperse. In certain embodiments, lipid coated nanoparticles generally range in size from greater than about 8-10 nm to about 5 μm in diameter, e.g., about 20-nm-3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm. As discussed above, the lipid coated nanoparticle population is considered monodisperse based upon the mean or median diameter of the population of lipid coated nanoparticles. Size is very important to therapeutic and diagnostic aspects as particles smaller than about 8-nm diameter are excreted through kidneys, and those particles larger than about 200 nm are often trapped by the liver and spleen. Thus, an embodiment on smaller monosized lipid coated nanoparticles are provided of less than about 150 nm for drug delivery and diagnostics in the patient or subject.

In certain embodiments, lipid coated nanoparticles are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-30 nm; they can be monosized or bimodal or graded-they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g., if a templating moiety in the aerosol process is not used. They could also be enlarged to macropores, i.e., 50-nm in diameter.

Pore surface chemistry of the nanoparticle material can be very diverse-all organosilanes yielding cationic, anionic, hydrophilic, hydrophobic, reactive groups-pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

In certain embodiments, the surface area of nanoparticles, as measured by the N2 BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the lipid coated nanoparticles are loaded with cargo to a capacity up to over 100 weight %: defined as (cargo weight/weight of lipid coated nanoparticle)×100. The optimal loading of cargo is often about 0.01 to 30% but this depends on the drug or drug combination which is incorporated as cargo into the lipid coated nanoparticle. This is generally expressed in μM of cargo per 10¹⁰ particles where values often ranging from 2000-100 μM per 10¹⁰ particles are used. Exemplary lipid coated nanoparticles exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the lipid coated nanoparticles according to one embodiment, the surface area is mainly internal as opposed to the external geometric surface area of the nanoparticle. The lipid bilayer supported on the porous particle according to one embodiment has a lower melting transition temperature, e.g., is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

The lipid bilayer may vary significantly in composition. Ordinarily, any lipid or polymer used in liposomes may also be used in lipid coated nanoparticles. Exemplary lipids are as otherwise described herein. Particular lipid bilayers for use in lipid coated nanoparticles comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the lipid coated nanoparticle. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of lipid coated nanoparticles according to an embodiment are that they are stable at pH 7, i.e., they don't leak their cargo, but at pH 5.5, which is that of the endosome lipid or polymer coating becomes destabilized initiating cargo release. This pH-triggered release is important for maintaining stability of the lipid coated nanoparticle up until the point that it is internalized in the cell by endocytosis, whereupon several pH triggered events cause release into the endosome and consequently, the cytosol of the cell. The lipid coated nanoparticle core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted lipid coated nanoparticles illicit only a weak immune response, because they do not support T-Cell help required for higher affinity IgG, a favorable result.

Lipid coated nanoparticles may exhibit at least one or more a number of characteristics (depending upon the embodiment) which distinguish them from prior art lipid coated nanoparticles: 1) In contrast to the prior art, an embodiment specifies monosized nanoparticles whose average size (diameter) is less than about 200-nm—this size is engineered to enable efficient cellular uptake by receptor mediated endocytosis and to minimize binding and uptake by non-target cells and organs; 2) Monodisperse sizes to enable control of biodistribution of the lipid coated nanoparticles; 3) To targeted nanoparticles that bind selected to cells based upon the inclusion of a targeting species on the lipid coated nanoparticle; 4) To targeted nanoparticles that induce receptor mediated endocytosis; 5) Induces dispersion of cargo into cytoplasm of targeted cells through the inclusion of fusogenic or endosomolytic peptides; 6) Provides particles with pH triggered release of cargo; 7) Exhibits controlled time dependent release of cargo (via extent of thermally induced crosslinking of silica nanoparticle matrix); 8) Exhibit time dependent pH triggered release; 9) Contain and provide cellular delivery of complex multiple cargoes; 10) Cytotoxicity of target cancer cells; 11) Diagnosis of target cancer cells; 12) Selective entry of target cells; 13) Selective exclusion from off-target cells (selectivity); 14) Enhanced fluidity of the supported lipid bilayer; 15) Sub-nanomolar and controlled binding affinity to target cells; 16) Sub-nanomolar binding affinity with targeting ligand densities; and/or 17) Colloidal and storage stability of compositions comprising lipid coated nanoparticles.

Various embodiments provide nanostructures which are constructed from nanoparticles which support a lipid bilayer(s). In some embodiments, the nanostructures include, for example, a core-shell structure including a porous particle core surrounded by a shell of lipid bilayer(s). The nanostructure, e.g., a porous silica nanostructure as described above, supports the lipid bilayer membrane structure.

In some embodiments, the lipid bilayer of the lipid coated nanoparticles can provide biocompatibility and can be modified to possess targeting species including, for example, targeting peptides, fusogenic peptides, antibodies, aptamers, and PEG (polyethylene glycol) to allow, for example, further stability of the lipid coated nanoparticles and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 40 to 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bilayer.

Numerous lipids which are used in liposome delivery systems may be used to form the lipid bilayer on nanoparticles to provide lipid coated nanoparticles. Virtually any lipid or polymer which is used to form a liposome or polymersome may be used in the lipid bilayer which surrounds the nanoparticles to form lipid coated nanoparticles according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bilayer of lipid coated nanoparticles according to an embodiment. Often cholesterol is incorporated into lipid bilayers of lipid coated nanoparticles in order to enhance structural integrity of the bilayer. These lipids are all readily available commercially from Avanti Polar Lipids, Inc. (Alabaster, Alabama, USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycaprolactone (PCL), polyanhydrides, poly(ortho) esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

Core-shell nanoparticles comprise a core and shell. The core, in one embodiment, comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer; see also Pluedemann, 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosures of which are incorporated herein by reference.

In certain embodiments of a lipid coated nanoparticle, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0]having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a lipid coated nanoparticle with a surface zeta potential which is neutral or close to neutral in character.

In other embodiments: (a) the lipid bilayer is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0]having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid bilayer comprises one or more PEG-containing phospholipids, for example 1,2-dioleoyl-sn-glycero-3-phosphoethanolamine-N-[methoxy (polyethylene glycol)](ammonium salt) (DOPE-PEG), 1,2-distearoyl-sn-glycero-3-phosphoethanolamine-N-[methoxy (polyethylene glycol)](ammonium salt) (DSPE-PEG), 1,2-distearoyl-sn-glycero-3-phosphoethanolamine-N-[amino (polyethylene glycol)](DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy (polyethylene glycol)-2000](ammonium salt) (DOPE-PEG₂₀₀₀), 1,2-distearoyl-sn-glycero-3-phosphoethanolamine-N-[methoxy (polyethylene glycol)-2000](ammonium salt) (DSPE-PEG₂₀₀₀), 1,2-distearoyl-sn-glycero-3-phosphoethanolamine-N-[amino (polyethylene glycol)-2000] (DSPE-PEG₂₀₀₀—NH₂) which can be used to covalent bind a functional moiety to the lipid bilayer.

In one illustrative embodiment of a lipid coated nanoparticle: (a) the one or more pharmaceutically-active agents include at least one anti-cancer agent; (b) less than around 10% to around 20% of the anti-cancer agent is released from the porous nanoparticulates in the absence of a reactive oxygen species; and (c) upon disruption of the lipid bilayer as a result of contact with a reactive oxygen species, the porous nanoparticulates release an amount of anti-cancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anti-cancer agent that would have been released had the lipid bilayer been lysed with 5% (w/V) Triton X-100.

One illustrative embodiment of a lipid coated nanoparticle comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3)N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; (b) are loaded with a siRNA or ricin toxin A-chain; and (c) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

Monosized lipid coated nanoparticles can comprise a wide variety of pharmaceutically-active ingredients such as nucleic acid, e.g., DNA.

The term “nuclear localization sequence” refers to a peptide sequence incorporated or otherwise crosslinked into histone proteins which comprise the histone-packaged supercoiled plasmid DNA. In certain embodiments, lipid coated nanoparticles may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR [PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998, TIBS, 23, 185-9 (1998); and Murat Cokol et al., “Finding nuclear localization signals”, at the website ubic.bioc.columbia.edu/papers/2000 nls/paper.html #tab2.

In general, lipid coated nanoparticles are biocompatible. Drugs and other cargo components are often loaded by adsorption and/or capillary filling of the pores of the particle core up to approximately 50% by weight of the final lipid coated nanoparticle (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

The porous nanoparticle core used to prepare the lipid coated nanoparticles can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In some aspects, the lipid bilayer is fused onto the porous particle core to form the monosized lipid coated nanoparticles. Lipid coated nanoparticles can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare lipid coated nanoparticles are monosized liposomes which can be prepared, for example, by extrusion of liposomes prepared by bath sonication through a filter with pore size of, for example, about 100 nm, using standard protocols known in the art or as otherwise described herein. Alternatively, the monosized liposomes are prepared from lipids using bath and probe sonication without extrusion. While the majority of the monosized liposomes are unilamellar when prepared using extrusion, in the absence of extrusion, the monosized liposomes will have an appreciable percent of multilamellar liposomes. The monosized liposomes can then be fused with the porous particle cores, for example, by sonicating (e.g., bath sonication, other) a mixtures of monosized liposomes and MSNs in buffered saline solution (e.g., PBS), followed by separation (centrifugation) and redispersing the pelleted lipid coated nanoparticles via sonication in a saline or other solution. In exemplary embodiments, excess amount of liposome (e.g., at least twice the amount of liposome to MSN) is used. To improve the lipid coated nanoparticle colloidal and/or storage stability of the lipid coated nanoparticle composition, the transition melting temperature (Tm) of the lipid bilayer should be greater than the temperature at which the lipid coated nanoparticles are to be stored and/or used. For storage stable liposomes, the inclusion of appreciable amounts of saturated phospholipids in the lipid bilayer is often used to increase the Tm of the lipid bilayer.

In certain diagnostic embodiments, various dyes or fluorescent (reporter) molecules can be included in the lipid coated nanoparticle cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed lipid coated nanoparticle can then be observed by, for example, confocal fluorescence for use in diagnostic applications. In addition, as discussed herein, plasmid DNA can be used as cargo in lipid coated nanoparticles, such that the plasmid may express one or more fluorescent proteins such as fluorescent green protein or fluorescent red protein which may be used in diagnostic applications.

In various embodiments, the lipid coated nanoparticle is used in a synergistic system where the lipid bilayer fusion or liposome fusion (i.e., on the porous particle core) is loaded and sealed with various cargo components with the pores (e.g., mesopores) of the particle core, thus creating a loaded lipid coated nanoparticle useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final lipid coated nanoparticle

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or anti-viral drugs such as anti-HBV or anti-HCV drugs), peptides, proteins, antibodies, DNA (especially plasmid DNA, including the exemplary histone-packaged super coiled plasmid DNA), RNAs (including shRNA and siRNA (which may also be expressed by the plasmid DNA incorporated as cargo within the lipid coated nanoparticles) fluorescent dyes, including fluorescent dye peptides which may be expressed by the plasmid DNA incorporated within the lipid coated nanoparticle.

In some embodiments, the cargo can be loaded into the pores (mesopores) of the porous particle cores to form the loaded lipid coated nanoparticle. In various embodiments, any conventional technology that is developed for liposome-based drug delivery, for example, targeted delivery using PEGylation, can be transferred and applied to the lipid coated nanoparticles.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded lipid coated nanoparticle having a concentration exceed about 100 times as compared with the charged cargo components in a solution. In other embodiments, by varying the charge of the mesoporous particle and the lipid bilayer, positively charged cargo components can be readily loaded into lipid coated nanoparticles.

Once produced, the loaded lipid coated nanoparticles can have a cellular uptake for cargo delivery into a desirable site after administration. For example, the cargo-loaded lipid coated nanoparticles can be administered to a patient or subject and the lipid coated nanoparticle comprising a targeting peptide can bind to a target cell and be internalized or uptaken by the target cell, for example, a cancer cell in a subject or patient. Due to the internalization of the cargo-loaded lipid coated nanoparticles in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA for example modified with a nuclear localization sequence can be directly delivered or internalized by the targeted cells. Thus, the DNA or RNA can be loaded first into a lipid coated nanoparticle and then into then through the target cells through the internalization of the loaded lipid coated nanoparticles.

As discussed, the cargo loaded into and delivered by the lipid coated nanoparticle to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded lipid coated nanoparticles can deliver their cargo into targeted cells for therapy or diagnostics.

In various embodiments, the lipid coated nanoparticles and/or the loaded lipid coated nanoparticles can provide a targeted delivery methodology for selectively delivering the lipid coated nanoparticles or the cargo components to targeted cells (e.g., cancer cells). For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a MET binding peptide as otherwise described herein.

For example, by providing a targeting active species (e.g., a targeting peptide) on the surface of the loaded lipid coated nanoparticle, the lipid coated nanoparticle selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bilayer, a large number of the cargo-loaded lipid coated nanoparticles can be recognized and internalized by this specific cancer cells due to the specific targeting of the exemplary SP94 or a MET or a CRLF2 binding peptide with the cancer (including liver) cells. In most instances, if the lipid coated nanoparticles are conjugated with the targeting peptide, the lipid coated nanoparticles will selectively bind to the cancer cells and no appreciable binding to the non-cancerous cells occurs.

Once bound and taken up by the target cells, the loaded lipid coated nanoparticles can release cargo components from the porous particle and transport the released cargo components into the target cell. For example, sealed within the lipid coated nanoparticle by the liposome fused bilayer on the porous particle core, the cargo components can be released from the pores of the lipid bilayer, transported across the lipid coated nanoparticle membrane of the lipid bilayer and delivered within the targeted cell. In embodiments, the release profile of cargo components in lipid coated nanoparticles can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the lipid coated nanoparticles can be pH-dependent.

In certain embodiments, the pH value for cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the lipid coated nanoparticle, the release of cargo can be relative short (a few hours to a day or so) or span for several days to about 20-30 days or longer. Thus, the lipid coated nanoparticle compositions may accommodate immediate release and/or sustained release applications from the lipid coated nanoparticles themselves.

In certain embodiments, the inclusion of surfactants can be provided to rapidly rupture the lipid bilayer, transporting the cargo components across the lipid bilayer of the lipid coated nanoparticle as well as the targeted cell. In certain embodiments, the phospholipid bilayer of the lipid coated nanoparticles can be ruptured by the application/release of a surfactant such as sodium dodecyl sulfate (SDS), among others to facilitate a rapid release of cargo from the lipid coated nanoparticle into the targeted cell. Other than surfactants, other materials can be included to rapidly rupture the bilayer. One example would be gold or magnetic nanoparticles that could use light or heat to generate heat thereby rupturing the bilayer. Additionally, the bilayer can be tuned to rupture in the presence of discrete biophysical phenomena, such as during inflammation in response to increased reactive oxygen species production. In certain embodiments, the rupture of the lipid bilayer can in turn induce immediate and complete release of the cargo components from the pores of the particle core of the lipid coated nanoparticles. In this manner, the lipid coated nanoparticle platform can provide an increasingly versatile delivery system as compared with other delivery systems in the art. For example, when compared to delivery systems using nanoparticles only, the disclosed lipid coated nanoparticle platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the lipid coated nanoparticle platform can provide a more stable system and can take advantage of the mesoporous core to control the loading and/or release profile and provide increased cargo capacity.

In addition, the lipid bilayer and its fusion on porous particle core can be fine-tuned to control the loading, release, and targeting profiles and can further comprise fusogenic peptides and related peptides to facilitate delivery of the lipid coated nanoparticles for greater therapeutic and/or diagnostic effect. Further, the lipid bilayer of the lipid coated nanoparticles can provide a fluidic interface for ligand display and multivalent targeting, which allows specific targeting with relatively low surface ligand density due to the capability of ligand reorganization on the fluidic lipid interface. Furthermore, the disclosed lipid coated nanoparticles can readily enter targeted cells while empty liposomes without the support of porous particles cannot be internalized by the cells.

Pharmaceutical compositions may comprise an effective population of lipid coated nanoparticles as otherwise described herein formulated to effect an intended result (e.g., therapeutic result and/or diagnostic analysis, including the monitoring of therapy) formulated in combination with a pharmaceutically acceptable carrier, additive or excipient. The lipid coated nanoparticles within the population of the composition may be the same or different depending upon the desired result to be obtained. Pharmaceutical compositions may also comprise an addition bioactive agent or drug, such as an anti-cancer agent or an anti-viral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The disclosure contemplates immediate and/or sustained/controlled release compositions, including compositions which comprise both immediate and sustained release formulations. This is particularly true when different populations of lipid coated nanoparticles are used in the pharmaceutical compositions or when additional bioactive agent(s) are used in combination with one or more populations of lipid coated nanoparticles as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 95%, about 0.25% to about 85%, about 0.5% to about 75% by weight of a compound/composition or compounds/compositions, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of lipid coated nanoparticles (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or would be apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic lipid coated nanoparticles and optionally at least one additional bioactive (e.g., anti-viral) agent.

Diagnostic methods may comprise administering to a patient in need (a patient suspected of having cancer) an effective amount of a population of diagnostic lipid coated nanoparticles (e.g., lipid coated nanoparticles which comprise a target species, such as a targeting peptide which binds selectively to cancer cells and a reporter component to indicate the binding of the lipid coated nanoparticles to cancer cells if the cancer cells are present) whereupon the binding of lipid coated nanoparticles to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic lipid coated nanoparticles (e.g., lipid coated nanoparticles which comprise a target species, such as a targeting peptide which binds selectively to cancer cells or other target cells and a reporter component to indicate the binding of the lipid coated nanoparticles to cancer cells if the cancer cells are present) to a patient or subject prior to treatment, determining the level of binding of diagnostic lipid coated nanoparticles to target cells in said patient and during and/or after therapy, determining the level of binding of diagnostic lipid coated nanoparticles to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Composition Embodiments

Historically, vaccines have worked by eliciting long lived soluble antibody production. These B cell vaccines are capable of neutralizing or blocking the spread of pathogens in the body. This long lived antibody response primarily targets and neutralizes pathogens as they are spreading from cell to cell, however, they are less effective at eliminating the pathogen once it has entered the host cell. On the other hand, T cell vaccines generate a population of immune cells capable of identifying infected cells and, through affinity dependent mechanisms, kill the cell; thereby eliminating pathogen production at its source. The CD4+ T cells activate innate immune cells, promote B cell antibody production, and provide growth factors and signals for CD8+ T cell maintenance and proliferation. The CD8+ T cells directly recognize and kill virally infected host cells. The ultimate goal of a T cell vaccine is to develop long lived CD8+ memory T cells capable of rapid expansion to combat microbial, e.g., viral, infection.

In some embodiments of a vaccine, a lipid coated nanoparticle includes a porous nanoparticle core which is made of a material comprising silica, polystyrene, alumina, titania, zirconia, or generally metal oxides, organometallates, organosilicates or mixtures thereof. A porous spherical silica nanoparticle core is used for the exemplary lipid coated nanoparticles and is surrounded by a supported lipid or polymer bilayer or multi-layer (multilamellar). Various embodiments provide nanostructures and methods for constructing and using the nanostructures and providing lipid coated nanoparticles. Porous silica particles are often used and are of varying sizes ranging in size (diameter) from less than 5 nm to 200 nm or 500 nm or more are readily available in the art or can be readily prepared using methods known in the art or alternatively, can be purchased from Melorium Technologies, Rochester, New York SkySpring Nanomaterials, Inc., Houston, Texas, USA or from Discovery Scientific, Inc., Vancouver, British Columbia. Multimodal silica nanoparticles may be readily prepared using the procedure of Carroll et al., 2009. Lipid coated nanoparticles can be readily obtained using methodologies known in the art. Lipid coated nanoparticles may be readily prepared, including lipid coated nanoparticles comprising lipids which are fused to the surface of the silica nanoparticle. See, for example, Liu et al. (2009), Liu et al. (2009), Liu et al. (2009), Lu et al. (1999). Other lipid coated nanoparticles for use are prepared according to the procedures which are presented in Ashley et al. (2010), Lu et al., (1999), Caroll et al., (2009), and as otherwise presented in the experimental section which follows. Multilamellar lipid coated nanoparticles may be prepared according to the procedures which are set forth in Moon et al., (2011), among others well known in the art. Another approach would be to hydrate lipid films and bath sonicate (without extrusion) and use polydisperse liposome fusion onto monodisperse cores loaded with cargo.

In some embodiments of the vaccine, the lipid coated nanoparticles include a core-shell structure which comprises a porous particle core surrounded by a shell of lipid which is often a multi-layer (multilamellar), but may include a single bilayer (unilamellar), (see Liu et al., 2009). The porous particle core can include, for example, a porous nanoparticle made of an inorganic and/or organic material as set forth above surrounded by a lipid bilayer. In some embodiments of the vaccine, the porous particle core of the lipid coated nanoparticles can be loaded with various desired species (“cargo”), especially including plasmid DNA which encodes for a microbial protein such as a bacterial protein, e.g., for a vaccine for tetanus, anthrax, haemophilus, pertussis, diphtheria, cholera, lyme disease, bacterial meningitis, Streptococcus pneumoniae, and typhoid, fungal protein, protist protein, archaea protein or a viral protein (fused to ubiquitin or not) or other microbial antigen (each of which may be ubiquitinylated) and additionally, depending upon the ultimate therapeutic goal, small molecules bioactive agents (e.g., antibiotics and/or anti-cancer agents as otherwise such as adjuvants as described herein), large molecules (e.g., especially including plasmid DNA, other macromolecules such as RNA, including small interfering RNA or siRNA or small hairpin RNA or shRNA or a polypeptide. In certain aspects, the lipid coated nanoparticles are loaded with super-coiled plasmid DNA, which can be used to deliver the microbial protein or optionally, other macromolecules such as a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-microbial agents and/or anti-cancer agents, nucleic acids (DNA and RNA, including siRNA and shRNA and plasmids which, after delivery to a cell, express one or more polypeptides, especially a full length microbial protein, e.g., fused to ubiquitin as a fusion protein or RNA molecules), such as for a particular purpose, as an immunogenic material which may optionally include a further therapeutic application or a diagnostic application.

In some embodiments, the lipid bilayer of the lipid coated nanoparticles can provide biocompatibility and can be modified to possess targeting species including, for example, targeting peptides including oligopeptides, antibodies, aptamers, and PEG (polyethylene glycol) (including PEG covalently linked to specific targeting species), among others, to allow, for example, further stability of the lipid coated nanoparticles and/or a targeted delivery into an antigen presenting cell (APC).

The lipid coated nanoparticle particle size distribution, according to the vaccine embodiment, depending on the application and biological effect, may be monodisperse or polydisperse. The silica cores can be rather monodisperse (i.e., a uniform sized population varying no more than about 5% in diameter e.g., +10-nm for a 200 nm diameter lipid coated nanoparticle especially if they are prepared using solution techniques) or rather polydisperse (i.e., a polydisperse population can vary widely from a mean or medium diameter, e.g., up to +200-nm or more if prepared by aerosol. Polydisperse populations can be sized into monodisperse populations. All of these are suitable for lipid coated nanoparticle formation. Lipid coated nanoparticles may be no more than about 500 nm in diameter, e.g., no more than about 200 nm in diameter in order to afford delivery to a patient or subject and produce an intended therapeutic effect. The pores of the lipid coated nanoparticles may vary in order to load plasmid DNA and/or other macromolecules into the core of the lipid coated nanoparticle. These may be varied pursuant to methods which are well known in the art.

Lipid coated nanoparticles according to the vaccine embodiment generally range in size from greater than about 8-10 nm to about 5 μm in diameter, about 20-nm-3 μm in diameter, about 10 nm to about 500 nm, or about 20-200-nm (including about 150 nm, which may be a mean or median diameter). As discussed above, the lipid coated nanoparticle population may be considered monodisperse or polydisperse based upon the mean or median diameter of the population of lipid coated nanoparticles. Size is very important to immunogenic aspects as particles smaller than about 8-nm diameter are excreted through kidneys, and those particles larger than about 200 nm are often trapped by the liver and spleen. Thus, an embodiment focuses in smaller sized lipid coated nanoparticles for drug delivery and diagnostics in the patient or subject. Lipid coated nanoparticles may be characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Pore sizes of mesopores range from about 2-30 nm; they can be monosized or bimodal or graded-they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) may be ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm, e.g., if a templating moiety in the aerosol process is not used. They could also be enlarged to macropores, i.e., 50-nm in diameter.

Pore surface chemistry of the nanoparticle material can be very diverse-all organosilanes yielding cationic, anionic, hydrophilic, hydrophobic, reactive groups-pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions, as further explained below.

The surface area of nanoparticles, as measured by the N2 BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the lipid coated nanoparticles are loaded with cargo to a capacity up to about 50 weight %: defined as (cargo weight/weight of loaded lipid coated nanoparticle)×100. The optimal loading of cargo is often about 0.01 to 10% but this depends on the drug or drug combination which is incorporated as cargo into the lipid coated nanoparticle. This is generally expressed in μM of cargo per 10¹⁰ lipid coated nanoparticle particles with values ranging, for example, from 2000-100 μM per 10¹⁰ particles. Exemplary lipid coated nanoparticles exhibit release of cargo at pH about 5.5, which is that of the endosome, but are stable at physiological pH of 7 or higher (7.4). The surface area of the internal space for loading is the pore volume whose value ranges from about 1.1 to 0.5 cubic centimeters per gram (cc/g). Note that in the lipid coated nanoparticles according to one embodiment, the surface area is mainly internal as opposed to the external geometric surface area of the nanoparticle. The lipid bilayer supported on the porous particle according to one embodiment has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In some embodiments, the lipid bilayer may vary significantly in composition. Ordinarily, any lipid or polymer which may be used in liposomes may also be used in lipid coated nanoparticles. Exemplary lipids are as otherwise described herein.

Particular lipid bilayers for use in lipid coated nanoparticles comprise mixtures of lipids (as otherwise described herein).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the lipid coated nanoparticle. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS or other silica amine incorporated into the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of lipid coated nanoparticles are that they are stable at pH 7, i.e., they don't leak their cargo, but at pH 5.5, which is that of the endosome lipid or polymer coating becomes destabilized initiating cargo release. This pH-triggered release is important for maintaining stability of the lipid coated nanoparticle up until the point that it is internalized in the cell by endocytosis, whereupon several pH triggered events cause release into the endosome and consequently, the cytosol of the cell. Quantitative experimental evidence has shown that targeted lipid coated nanoparticles illicit only a weak immune response in the absence of the components which are incorporated into lipid coated nanoparticles, because they do not support T-Cell help required for higher affinity IgG, a favorable result.

Various embodiments provide nanostructures which are constructed from nanoparticles which support a lipid bilayer(s). In embodiments according to the vaccine, the nanostructures may include, for example, a core-shell structure including a porous particle core surrounded by a shell of lipid bilayer(s). The nanostructure, e.g., a porous silica nanostructure as described above, supports the lipid bilayer membrane structure. In some embodiments, the lipid bilayer of the lipid coated nanoparticles can provide biocompatibility and can be modified to possess targeting species including, for example, targeting peptides, antibodies, aptamers, and PEG (polyethylene glycol) linked to targeting species to allow, for example, further stability of the lipid coated nanoparticles and/or a targeted delivery into a bioactive cell, in particular an APC. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bilayer.

Numerous lipids which are used in liposome delivery systems may be used to form the lipid bilayer on nanoparticles to provide lipid coated nanoparticles. Virtually any lipid or polymer which is used to form a liposome or polymersome may be used in the lipid bilayer which surrounds the nanoparticles to form lipid coated nanoparticles according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol is included as a lipid. Often cholesterol is incorporated into lipid bilayers of lipid coated nanoparticles in order to enhance structural integrity of the bilayer. These lipids are all readily available commercially from Avanti Polar Lipids, Inc. (Alabaster, Alabama, USA). DOPE and DPPE are particularly useful for conjugating (through an appropriate crosslinker) peptides, polypeptides, including antibodies, RNA and DNA through the amine group on the lipid.

In certain embodiments, the nanoparticulate cores can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycaprolactone (PCL), polyanhydrides, poly(ortho) esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

In still other embodiments, the lipid coated nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the lipid coated nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

The cores can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. In some embodiments, the cores have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In one embodiment, the cores are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

In certain embodiments, the core-shell nanoparticles comprise a core and shell. The core comprises silica and an optional absorber molecule. The absorber molecule is incorporated into the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like. The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patents applications Ser. Nos. 10/306,614, 10/536,569, the disclosure of such processes therein are incorporated herein by reference.

In certain embodiments, the lipid bilayer is comprised of one or more lipids selected from the group consisting of phosphatidyl-cholines (PCs) and cholesterol. In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0]having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0]having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%. In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In one embodiment a lipid coated nanoparticle which is included in compositions may include at least one anti-cancer agent, especially an anti-cancer agent which treats a cancer which occurs secondary to a viral infection.

One illustrative embodiment of a lipid coated nanoparticle comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3)N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; and (b) are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

Lipid coated nanoparticles can comprise a wide variety of pharmaceutically-active ingredients.

In certain embodiments, the lipid coated nanoparticles may include a reporter for diagnosing a disease state or condition. The term “reporter” is used to describe an imaging agent or moiety which is incorporated into the phospholipid bilayer or cargo of lipid coated nanoparticles according to an embodiment and provides a signal which can be measured. The moiety may provide a fluorescent signal or may be a radioisotope which allows radiation detection, among others. Exemplary fluorescent labels for use in lipid coated nanoparticles (e.g., via conjugation or adsorption to the lipid bilayer or silica core, although these labels may also be incorporated into cargo elements such as DNA, RNA, polypeptides and small molecules which are delivered to cells by the lipid coated nanoparticles, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of a disease state in a patient and or the progress of therapy in a patient or subject.

The term “histone-packaged supercoiled plasmid DNA” is used to describe a component of lipid coated nanoparticles which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the lipid coated nanoparticles). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into lipid coated nanoparticles (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced. The DNA may also be double stranded linear DNA, instead of plasmid DNA, which also may be optionally supercoiled and/or packaged with histones or other packaging components.

In certain embodiments, lipid coated nanoparticles comprise a plasmid (which may be a histone-packaged supercoiled plasmid DNA) which encodes an anti-microbial or a microbial protein, e.g., viral protein, antigen often complexed with ubiquitin protein (e.g., as a fusion protein). The plasmid, including a histone-packaged supercoiled plasmid DNA, may be modified (crosslinked) with a nuclear localization sequence (note that the histone proteins may be crosslinked with the nuclear localization sequence or the plasmid itself can be modified to express a nuclear localization sequence) in order to enhance the ability of the histone-packaged plasmid to penetrate the nucleus of a cell and deposit its contents there (to facilitate expression and ultimately cell death). These peptide sequences assist in carrying the histone-packaged plasmid DNA and the associated histones into the nucleus of a cell to facilitate expression and antigen presentation. Any number of crosslinking agents, well known in the art and as otherwise described herein, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell. In alternative embodiments, the DNA plasmid is included in the absence of histone packaging and/or a nuclear localization sequence and the plasmid expresses a microbial protein (e.g., full length viral protein) in the cytosol of the cell (APC) to which the lipid coated nanoparticle is delivered.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR [PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998 and Murat Cokol et al., “Finding nuclear localization signals”, at the website ubic.bioc.columbia.edu/papers/2000 nls/paper.html #tab2.

Viruses that may raise an immunogenic response include any viral bioagent which is an animal virus. Viruses which affect animals, include, for example, Papovaviruses, e.g., polyoma virus and SV40; Poxviruses, e.g., vaccinia virus and variola (smallpox); Adenoviruses, e.g., human adenovirus; Herpesviruses, e.g., Human Herpes Simplex types I and II; Parvoviruses, e.g., adeno associated virus (AAV); Reoviruses, e.g., rotavirus and reovirus of humans; Picornaviruses, e.g., poliovirus; Togaviruses, including the alpha viruses (group A), e.g., Sindbis virus and Semliki forest virus (SFV) and the flaviviruses (group B), e.g., dengue virus, yellow fever virus and the St. Louis encephalitis virus; Retroviruses, e.g., HIV I and II, Rous sarcoma virus (RSV), and mouse leukemia viruses; Rhabdoviruses, e.g., vesicular stomatitis virus (VSV) and rabies virus; Paramyxoviruses, e.g., mumps virus, measles virus and Sendai virus;

Arena viruses, e.g., lassa virus; Bunyaviruses, e.g., bunyamwera (encephalitis); Coronaviruses, e.g., common cold, GI distress viruses, Orthomyxovirus, e.g., influenza; Caliciviruses, e.g., Norwalk virus, Hepatitis E virus; Filoviruses, e.g., ebola virus and Marburg virus; and Astroviruses, e.g., astrovirus, among others.

Virus such as Sin Nombre virus, Nipah virus, Influenza (especially H5N1 influenza), Herpes Simplex Virus (HSV1 and HSV-2), Coxsackie virus, Human immunodeficiency virus (I and II), Andes virus, Dengue virus, Papilloma, Epstein-Barr virus (mononucleosis), Variola (smallpox) and other pox viruses and West Nile virus, among numerous others viruses.

A short list of animal viruses which are particularly relevant includes the following viruses: Reovirus, Rotavirus, Enterovirus, Rhinovirus, Hepatovirus, Cardiovirus, Aphthovirus, Parechovirus, Erbovirus, Kobuvirus, Teschovirus, Norwalk virus, Hepatitis E virus, Rubella virus, Lymphocytic choriomeningitis virus, HIV-1, HIV-2, HTLV (especially HTLV-1), Herpes Simplex Virus 1 and 2, Sin Nombre virus, Nipah virus, Coxsackie Virus, Dengue virus, Yellow fever virus, Hepatitis A virus, Hepatitis B virus, Hepatitis C virus, Influenzavirus A, B and C, Isavirus, Thogotovirus, Measles virus, Mumps virus, Respiratory syncytial virus, California encephalitis virus, Hantavirus, Rabies virus, Ebola virus, Marburg virus, Corona virus, Astrovirus, Borna disease virus, and Variola (smallpox virus).

In certain embodiments, compositions may include lipid coated nanoparticles which contain an anti-cancer agent as a co-therapy, but principally as a separate distinguishable population from immunogenic lipid coated nanoparticles otherwise described herein. In such an embodiment, lipid coated nanoparticles which target cancer cells and which contain an anti-cancer agent may be co-administered with immunogenic lipid coated nanoparticles.

APCs fall into two categories: professional and non-professional. T cells cannot recognize or respond to “free” antigen. Recognition by T cells occurs when an antigen has been processed and presented by APCs via carrier molecules like MHC and CD1 molecules. Most cells in the body can present antigen to CD8+ T cells via MHC class I molecules and, thus, act as “APCs”; however, the term is often limited to specialized cells that can prime T cells (i.e., activate a T cell that has not been exposed to antigen), termed a naive T cell. These professional APCs, in general, express MHC class II as well as MHC class I molecules, and can stimulate CD4+ “helper” T-cells as well as CD8+ “cytotoxic” T cells respectively. The cells that express MHC class II molecules are often referred to as professional antigen-presenting cells an include dendritic cells (DCs), macrophages, B-cells which express a B cell receptor (BCR) and specific antibody which binds to the BCR and certain activated epithelial cells. Professional APCs internalize antigens, generally by phagocytosis or by receptor-mediated endocytosis and then display a fragment of the antigen on the membrane surface of the cell through its binding to a class II MHC molecule. Non-professional APCs do not express the Major Histocompatibility Complex class II (MHC class II) proteins required for interaction with naïve T cells; these are only expressed upon stimulation of the non-professional APC by cytokines such as IFN-γ. All nucleated cells express MHC class I molecules and consequently all are considered non-professional APCs. Erythrocytes do not have a nucleus; consequently, they are one of the few cells in the body that cannot display antigens.

Compositions provide their principal immunological reaction through interaction with either professional APCs or non-professional APCs. Non-professional antigen presenting cells include virally infected cells and cancer cells.

In order to covalently link any of the fusogenic peptides or endosomolytic peptides to components of the lipid bilayer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, etc.) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition we could use click chemistry to modify the peptides with azide or alkyne for cu-catalyzed crosslinking, and we could also use a cu-free click chemistry reaction.

The plasmids described herein are used to express a microbial antigen (e.g., a viral protein). Optionally the antigen is in combination with ubiquitin as a fusion protein. In some embodiments, the plasmid vectors are adenoviral, lentiviral and/or retroviral vectors many, of which may readily accommodate the viral protein. Exemplary recombinant adenovirus vectors include those commercialized as the AdEasy™ System by many companies including Stratagene® (stratagene.com), QBiogene® (qbiogene.com), and the ATCC® (atcc. org). AdEasy™ vectors include pShuttle, pShuttle-CMV, and pAdEasy-1. The pAdEasy-1 vector is devoid of E1 and E3 regions so that the recombinant virus will not replicate in cells other than complementing cells, such as human embryonic kidney 293 (HEK293). These methods are described by He et al., Proc. Natl. Acad. Sci., USA, 95, pp. 2509-2514 (1998). An exemplary lentiviral expression system is the The ViraPower™ Lentiviral Expression System (Invitrogen, Carlsbad, California 92008, invitrogen.com) is loosely based on the HIV-1 strain NL4-3. Other commercial adenoviral, lentiviral and retroviral vectors are well known in the art.

The crystal structure of ubiquitin evidences two accessible lysine groups which are used with the crosslinker chemistry described above to anchor the ubiquitin to a component (e.g., viral protein or peptide or a lipid, phospholipid, other) of a lipid bilayer of the lipid coated nanoparticle. Ubiquitination does not have to occur in any specific part of the target peptide, it only acts as a marker to signal degradation. This is only intended to speed up the process; the cell would ubiquitinate a foreign peptide naturally delivering ubiquitinated microbial antigens potentially skip this step and speed up the process. Accordingly, ubiquitin is an optional element of the lipid coated nanoparticles.

As discussed in detail above, the porous nanoparticle core can include porous nanoparticles having at least one dimension, for example, a width or a diameter of about 3000 nm or less, about 1000 nm or less, about 500 nm or less, about 200 nm or less. In one embodiment, the nanoparticle core is spherical with an exemplary diameter of about 500 nm or less, e.g., about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, lipid coated nanoparticles are biocompatible. Drugs and other cargo components are often loaded by adsorption and/or capillary filling of the pores of the particle core up to approximately 50% by weight of the final lipid coated nanoparticle (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

The porous nanoparticle core used to prepare the lipid coated nanoparticles can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In exemplary aspects, the lipid bilayer is fused onto the porous particle core to form the lipid coated nanoparticle. Lipid coated nanoparticles can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare lipid coated nanoparticles can be prepared, for example, by extrusion of hydrated lipid films containing other components through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the lipid coated nanoparticle in order to improve the lipid coated nanoparticle colloidal stability.

In various embodiments, the lipid coated nanoparticle is used in a synergistic system where the lipid bilayer fusion or liposome fusion (i.e., on the porous particle core) is loaded and sealed with various cargo components with the pores (mesopores) of the particle core, thus creating a loaded lipid coated nanoparticle useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final lipid coated nanoparticle.

A fusion and synergistic loading mechanism can be included for cargo delivery. For example, cargo can be loaded, encapsulated, or sealed, synergistically through liposome fusion on the porous particles. In addition to microbial proteins, fusion proteins (e.g., viral proteins, including full length viral proteins and fusion proteins based upon viral proteins and ubiquitin) and/or plasmid vectors which can express microbial protein or micrbial protein fused with ubiquitin. The cargo can also include, for example, small molecule drugs (e.g., especially including anti-cancer drugs and/or anti-viral drugs such as anti-HBV or anti-HCV drugs), peptides, proteins, antibodies, DNA (other plasmid

DNA, RNAs (including shRNA and siRNA (which may also be expressed by plasmid DNA incorporated as cargo within the lipid coated nanoparticles), fluorescent dyes, including fluorescent dye peptides which may be expressed by the plasmid DNA incorporated within the lipid coated nanoparticle as reporters for diagnostic methods associated with establishing the mechanism of immunogenicity of lipid coated nanoparticles.

Loading of plasmid within the porous core may be difficult to achieve. One approach is to synthesize large pore particles; however, it is somewhat likely that the plasmid will interact with the exterior of the MSN core regardless of pore size. Therefore, modification of the MSN framework to incorporate cationic amine groups to form the core as described above will enhance the plasmid/MSN association due to electrostatic attraction (plasmid carries a net negative charge). Another approach would be to incorporate a small amount of cationic lipids (DOPE, DPPE, DSPE, DOTAP, etc.) into the bilayer formulation to encourage plasmid/MSN association.

Protein cargo loading can be electrostatically driven, cationic cores/net negative protein or anionic cores/net positive protein. It is possible to conjugate the protein to the MSN core using the previously mentioned conjugation strategies by modifying the core with amine, carboxylic acid, thiol, click chemistry, etc. We can also make better use of the pores since protein should be much smaller and more compact than the plasmid constructs. Another approach is to digest the protein into smaller pieces and load the particle with fragments of the protein.

In some embodiments, the cargo can be loaded into the pores (mesopores) of the porous particle cores to form the loaded lipid coated nanoparticle. In various embodiments, any conventional technology that is developed for liposome-based drug delivery, for example, targeted delivery using PEGylation, can be transferred and applied to the lipid coated nanoparticles.

As discussed above, electrostatics and pore size can play a role in cargo loading. For example, porous silica nanoparticles can carry a negative charge and the pore size can be tunable from about 2 nm to about 10 nm or more, e.g., 6, 7, 8, 9, 10, 11, 12, 13, 14, 15, 20 or up to 25 nm, or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively charged species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded lipid coated nanoparticle having a concentration exceed about 100 times as compared with the charged cargo components in a solution. In other embodiments, by varying the charge of the mesoporous particle and the lipid bilayer, positively charged cargo components can be readily loaded into lipid coated nanoparticles.

Once produced, the loaded lipid coated nanoparticles can have a cellular uptake for cargo delivery into a desirable site after administration. For example, the cargo-loaded lipid coated nanoparticles can be administered to a patient or subject and the lipid coated nanoparticle comprising a targeting peptide can bind to a target cell and be internalized by the target cell, for example, an APC in a subject or patient. Due to the internalization of the cargo-loaded lipid coated nanoparticles in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA, e.g., modified with a nuclear localization sequence, can be directly delivered or internalized by the targeted cells. Thus, the DNA or RNA can be loaded first into a lipid coated nanoparticle and then into then through the target cells through the internalization of the loaded lipid coated nanoparticles.

As discussed, the cargo loaded into and delivered by the lipid coated nanoparticle to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded lipid coated nanoparticles can deliver their cargo into targeted cells for therapy or diagnostics.

In various embodiments, the lipid coated nanoparticles and/or the loaded lipid coated nanoparticles can provide a targeted delivery methodology for selectively delivering the lipid coated nanoparticles or the cargo components to targeted cells (e.g., cancer cells). For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a peptide which targets APC or other cells as otherwise described herein.

For example, by providing a targeting active species (for example, a targeting peptide) on the surface of the loaded lipid coated nanoparticle, the lipid coated nanoparticle selectively binds to the targeted cell in accordance with the present teachings. In most instances, if the lipid coated nanoparticles are conjugated with the targeting peptide, the lipid coated nanoparticles will selectively bind to the cancer cells and no appreciable binding to the non-cancerous cells occurs.

Once bound and taken up by the target cells, the loaded lipid coated nanoparticles can release cargo components from the porous particle and transport the released cargo components into the target cell. For example, sealed within the lipid coated nanoparticle by the liposome fused bilayer on the porous particle core, the cargo components can be released from the pores of the lipid bilayer, transported across the lipid coated nanoparticle membrane of the lipid bilayer and delivered within the targeted cell. In embodiments, the release profile of cargo components in lipid coated nanoparticles can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the lipid coated nanoparticles can be pH-dependent.

In certain embodiments, the pKa for the cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the lipid coated nanoparticle, the release of cargo can be relative short (a few hours to a day or so) or span for several days to about 20-30 days or longer. Thus, the lipid coated nanoparticle compositions may accommodate immediate release and/or sustained release applications from the lipid coated nanoparticles themselves.

In certain embodiments, the inclusion of surfactants can be provided to rapidly rupture the lipid bilayer, transporting the cargo components across the lipid bilayer of the lipid coated nanoparticle as well as the targeted cell. In certain embodiments, the phospholipid bilayer of the lipid coated nanoparticles can be ruptured by the application/release of a surfactant such as sodium dodecyl sulfate (SDS), among others to facilitate a rapid release of cargo from the lipid coated nanoparticle into the targeted cell. Other than surfactants, other materials can be included to rapidly rupture the bilayer. One example would be gold or magnetic nanoparticles that could use light or heat to generate heat thereby rupturing the bilayer. Additionally, the bilayer can be tuned to rupture in the presence of discrete biophysical phenomena, such as during inflammation in response to increased reactive oxygen species production. In certain embodiments, the rupture of the lipid bilayer can in turn induce immediate and complete release of the cargo components from the pores of the particle core of the lipid coated nanoparticles. In this manner, the lipid coated nanoparticle platform can provide an increasingly versatile delivery system as compared with other delivery systems in the art. For example, when compared to delivery systems using nanoparticles only, the disclosed lipid coated nanoparticle platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the lipid coated nanoparticle platform can provide a more stable system and can take advantage of the mesoporous core to control the loading and/or release profile and provide increased cargo capacity.

In addition, the lipid bilayer and its fusion on porous particle core can be fine-tuned to control the loading, release, and targeting profiles and can further comprise fusogenic peptides and related peptides to facilitate delivery of the lipid coated nanoparticles for greater therapeutic and/or diagnostic effect. Further, the lipid bilayer of the lipid coated nanoparticles can provide a fluidic interface for ligand display and multivalent targeting, which allows specific targeting with relatively low surface ligand density due to the capability of ligand reorganization on the fluidic lipid interface. Furthermore, the disclosed lipid coated nanoparticles can readily enter targeted cells while empty liposomes without the support of porous particles cannot be internalized by the cells.

Exemplary multilamellar liposomes can be produced by the method of Moon, et al., “Interbilayer-crosslinked multilamellar vesicles as synthetic vaccines for potent humoral and cellular immune responses”, Nature Materials, 2011, 10, pp. 243-251 through crosslinking by divalent cation crosslinking with dithiol chemistry. Another approach would be to hydrate lipid films and bath sonicate (without extrusion) and use polydisperse liposome fusion onto monodisperse cores loaded with cargo.

Pharmaceutical compositions comprise an effective population of lipid coated nanoparticles as otherwise described herein formulated to effect an intended result (e.g., therapeutic result and/or diagnostic analysis, including the monitoring of therapy) formulated in combination with a pharmaceutically acceptable carrier, additive or excipient. The lipid coated nanoparticles within the population of the composition may be the same or different depending upon the desired result to be obtained. Pharmaceutical compositions may also comprise an addition bioactive agent or drug, such as an anti-cancer agent or an anti-microbial agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The present disclosure contemplates immediate and/or sustained/controlled release compositions, including compositions which comprise both immediate and sustained release formulations. This is particularly true when different populations of lipid coated nanoparticles are used in the pharmaceutical compositions or when additional bioactive agent(s) are used in combination with one or more populations of lipid coated nanoparticles as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of lipid coated nanoparticles (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic lipid coated nanoparticles and optionally at least one additional bioactive (e.g., anti-viral) agent.

Diagnostic methods comprise administering to a patient in need (a patient suspected of having cancer) an effective amount of a population of diagnostic lipid coated nanoparticles (e.g., lipid coated nanoparticles which comprise a target species, such as a targeting peptide which binds selectively to APC cells or virus infected cells and a reporter component to indicate the binding of the lipid coated nanoparticles to APC or virus infected cells if the infection is present) whereupon the binding of lipid coated nanoparticles to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic lipid coated nanoparticles (e.g., lipid coated nanoparticles which comprise a target species, such as a targeting peptide which binds selectively to APC cells or other target cells and a reporter component to indicate the binding of the lipid coated nanoparticles to the target cells) to a patient or subject prior to treatment, determining the level of binding of diagnostic lipid coated nanoparticles to target cells in said patient and during and/or after therapy, determining the level of binding of diagnostic lipid coated nanoparticles to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

In certain embodiments, lipid coated nanoparticles on are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded-they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2 nm in diameter) all the way down to about 0.03-nm e.g. if a templating moiety in the aerosol process is not used. They could also be enlarged to macropores, e.g., 50 nm in diameter.

Pore surface chemistry of the nanoparticle material can be very diverse-all organosilanes yielding cationic, anionic, hydrophilic, hydrophobic, reactive groups-pore surface chemistry, especially charge and hydrohobicity, affect loading capacity. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m2/g to >about 1200 m2/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the lipid coated nanoparticles are loaded with cargo to a capacity up to over 100 weight %: defined as (cargo weight/weight of lipid coated nanoparticle)×100. The optimal loading of cargo is often about 0.01 to 30% but this depends on the drug or drug combination which is incorporated as cargo into the lipid coated nanoparticle. This is generally expressed in μM per 10¹⁰ particles where we have values ranging from 2000-100 μM per 10¹⁰ particles. In one embodiment, lipid coated nanoparticles exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in certain lipid coated nanoparticles, the surface area is mainly internal as opposed to the external geometric surface area of the nanoparticle.

The lipid bilayer supported on the porous particle according to one embodiment has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In one embodiment, the lipid bilayer may vary significantly in composition. Ordinarily, any lipid or polymer which may be used in liposomes may also be used in lipid coated nanoparticles. In one embodiment, lipid bilayers for use in lipid coated nanoparticles comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the lipid coated nanoparticle. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely.

This in turn controls the release rate of the internal cargo. This occurs because molecules that are strongly attracted to the internal surface area of the pores diffuse slowly out of the particle cores, so dissolution of the particle cores controls in part the release rate.

Further characteristics of lipid coated nanoparticles are that they are stable at pH 7, i.e. they don't leak their cargo, but at pH 5.5, which is that of the endosome lipid or polymer coating becomes destabilized initiating cargo release. This pH-triggered release is important for maintaining stability of the lipid coated nanoparticle up until the point that it is internalized in the cell by endocytosis, whereupon several pH triggered events cause release into the endosome and consequently, the cytosol of the cell. The lipid coated nanoparticle core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted lipid coated nanoparticles illicit only a weak immune response, because they do not support T-Cell help required for higher affinity IgG, a favorable result.

Various embodiments provide nanostructures which are constructed from nanoparticles which support a lipid bilayer(s). In some embodiments, the nanostructures include, for example, a core-shell structure including a porous particle core surrounded by a shell of lipid bilayer(s). The nanostructure, e.g., a porous silica nanostructure as described above, supports the lipid bilayer membrane structure.

In some embodiments, the lipid bilayer of the lipid coated nanoparticles can provide biocompatibility and can be modified to possess targeting species including, for example, targeting peptides, fusogenic peptides, antibodies, aptamers, and PEG (polyethylene glycol) to allow, for example, further stability of the lipid coated nanoparticles and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20 about 5% to about 15%, or about 10% by weight of the lipids which are included in the lipid bilayer.

Numerous lipids which are used in liposome delivery systems may be used to form the lipid bilayer on nanoparticles to provide lipid coated nanoparticles. Virtually any lipid or polymer which is used to form a liposome or polymersome may be used in the lipid bilayer which surrounds the nanoparticles to form lipid coated nanoparticles according to an embodiment. In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-Oleoyl-2-[12-[(7-nitro-2-1, 3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of lipid coated nanoparticles according to an embodiment. Often cholesterol is incorporated into lipid bilayers of lipid coated nanoparticles in order to enhance structural integrity of the bilayer. These lipids are all readily available commercially from Avanti Polar Lipids, Inc. (Alabaster, Alabama, USA). DOPE and

DPPE are particularly useful for conjugating (through an appropriate crosslinker) peptides, polypeptides, including antibodies, RNA and DNA through the amine group on the lipid.

Pegylated phospholipids include for example, pegylated 1,2-distearoyl-sn-glycero-3-phosphoethanolamine (PEG-DSPE), pegylated 1,2-dioleoyl-sn-glycero-3-phosphoethanolamine (PEG-DOPE), pegylated 1,2-dipalmitoyl-sn-glycero-3-phosphoethanolamine (PEG-DPPE), and pegylated 1,2-dimyristoyl-sn-glycero-3-phosphoethanolamine (PEG-DMPE), among others, including a pegylated ceramide (e.g. N-octanoyl-sphingosine-1-succinylmethoxy-PEG or N-palmitoyl-sphingosine-1-succinylmethoxy-PEG, among others). The PEG generally ranges in size (average molecular weight for the PEG group) from about 350-7500, about 350-5000, about 500-2500, about 1000-2000. Pegylated phospholipids may comprise the entire phospholipid monolayer of hybrid phospholipid lipid coated nanoparticles, or alternatively they may comprise a minor component of the lipid monolayer or be absent. Accordingly, the percent by weight of a pegylated phospholipid in phospholipid monolayers ranges from 0% to 100% or 0.01% to 99%, e.g., about 5%, 10%, 15%, 20%, 25%, 30%, 35%, 40%, 50%, 55%, 60% and the remaining portion of the phospholipid monolayer comprising at least one additional lipid (such as cholesterol, usually in amounts less than about 50% by weight), including a phospholipid.

In certain embodiments, the porous nanoparticulates can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycarprolactone (PCL), polyanhydrides, poly(ortho) esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof. The silica nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known (see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982). The organo-silane can cause gels, so it may be desirable to employ an alcohol or other known stabilizers. Processes to synthesize core-shell nanoparticles using modified Stoeber processes can be found in U.S. patents applications Ser. Nos. 10/306,614 and 10/536,569, the disclosure of such processes therein are incorporated herein by reference.

In certain embodiments of a lipid coated nanoparticle, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0]having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1]. In other embodiments: (a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0]having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

Lipid coated nanoparticles can comprise a wide variety of pharmaceutically-active ingredients. The term “hydrophobic drug” or “hydrophobic active agent” is used to describe an active agent which is lipophilic/hydrophobic in nature. Exemplary lipophilic/hydrophobic drugs which are useful include, for example, analgesics and anti-inflammatory agents, such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; Anthelmintics, such as albendazole, bephenium hydroxynaphthoate, cambendazole, dichlorophen, ivermectin, mebendazole, oxamniquine, oxfendazole, oxantel embonate, praziquantel, pyrantel embonate, thiabendazole; Anti-arrhythmic agents such as amiodarone HCl, disopyramide, flecainide acetate, quinidine sulphate; Anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; Anti-coagulants such as dicoumarol, dipyridamole, nicoumalone, phenindione; Anti-depressants such as amoxapine, maprotiline HCl, mianserin HCL, nortriptyline HCl, trazodone HCL, trimipramine maleate; Anti-diabetics such as acetohexamide, chlorpropamide, glibenclamide, gliclazide, glipizide, tolazamide, tolbutamide; Anti-epileptics such as beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid; Anti-fungal agents such as amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid; Anti-gout agents such as allopurinol, probenecid, sulphin-pyrazone; Anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; Anti-malarials such as amodiaquine, chloroquine, chlorproguanil HCl, halofantrine HCl, mefloquine HCl, proguanil HCl, pyrimethamine, quinine sulphate; Anti-migraine agents such as dihydroergotamine mesylate, ergotamine tartrate, methysergide maleate, pizotifen maleate, sumatriptan succinate; Anti-muscarinic agents such as atropine, benzhexol HCl, biperiden, ethopropazine HCl, hyoscyamine, mepenzolate bromide, oxyphencylcimine HCl, tropicamide; Anti-neoplastic agents and Immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; Anti-protozoal agents such as benznidazole, clioquinol, decoquinate, diiodohydroxyquinoline, diloxanide furoate, dinitolmide, furzolidone, metronidazole, nimorazole, nitrofurazone, ornidazole, tinidazole; Anti-thyroid agents such as carbimazole, propylthiouracil; Anxiolytic, sedatives, hypnotics and neuroleptics such as alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone; β-Blockers such as acebutolol, alprenolol, atenolol, labetalol, metoprolol, nadolol, oxprenolol, pindolol, propranolol; Cardiac Inotropic agents such as amrinone, digitoxin, digoxin, enoximone, lanatoside C, medigoxin; Corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; Diuretics such as acetazolamide, amiloride, bendrofluazide, bumetanide, chlorothiazide, chlorthalidone, ethacrynic acid, frusemide, metolazone, spironolactone, triamterene; Anti-parkinsonian agents such as bromocriptine mesylate, lysuride maleate; Gastro-intestinal agents such as bisacodyl, cimetidine, cisapride, diphenoxylate HCl, domperidone, famotidine, loperamide, mesalazine, nizatidine, omeprazole, ondansetron HCL, ranitidine HCl, sulphasalazine; Histamine H,-Receptor Antagonists such as acrivastine, astemizole, cinnarizine, cyclizine, cyproheptadine HCl, dimenhydrinate, flunarizine HCl, loratadine, meclozine HCl, oxatomide, terfenadine; Lipid regulating agents such as bezafibrate, clofibrate, fenofibrate, gemfibrozil, probucol; Nitrates and other anti-anginal agents such as amyl nitrate, glyceryl trinitrate, isosorbide dinitrate, isosorbide mononitrate, pentaerythritol tetranitrate; Nutritional agents such as betacarotene, vitamin A, vitamin B2, vitamin D, vitamin E, vitamin K; Opioid analgesics such as codeine, dextropropyoxyphene, diamorphine, dihydrocodeine, meptazinol, methadone, morphine, nalbuphine, pentazocine; Sex hormones such as clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone; and Stimulants such as amphetamine, dexamphetamine, dexfenfluramine, fenfluramine, mazindol, among others. Other hydrophobic drugs include rapamycin, docetaxel, paclitaxel, carbazitaxel, thiazolidinediones (e.g., rosiglitazone, pioglitazone, lobeglitazone, troglitazone, netoglitazone, riboglitazone and ciglitazone) and curcumin, among others.

Targeting peptides are known in the art. Targeting peptides may be complexed or optionally, covalently linked to the lipid bilayer through use of a crosslinking agent as otherwise described herein.

In order to covalently link any of the fusogenic peptides or endosomolytic peptides to components of the lipid bilayer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, and the like) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition click chemistry may be used to modify the peptides with azide or alkyne for cu-catalyzed crosslinking, and we could also use a cu-free click chemistry reaction.

Exemplary crosslinking agents include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[ß-Maleimidopropionamido]hexanoate (SMPH), N-[ß-Maleimidopropionic acid]hydrazide (BMPH), NHS-(PEG)ₙ-maleimide, succinimidyl-[(N-maleimidopropionamido)-tetracosaethyleneglycol]ester (SM (PEG)₂₄), and succinimidyl 6-[3′-(2-pyridyldithio)-propionamido]hexanoate (LC-SPDP), among others.

As discussed in detail above, the porous nanoparticle core can include porous nanoparticles having at least one dimension, for example, a width or a diameter of about 3000 nm or less, about 1000 nm or less, about 500 nm or less, about 200 nm or less. In one embodiment, the nanoparticle core is spherical with a diameter of about 500 nm or less, or about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, lipid coated nanoparticles are biocompatible. Drugs and other cargo components are often loaded by adsorption and/or capillary filling of the pores of the particle core up to approximately 50% by weight of the final lipid coated nanoparticle (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In one embodiment, the porous nanoparticle core used to prepare the lipid coated nanoparticles can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In certain aspects, the lipid bilayer is fused onto the porous particle core to form the lipid coated nanoparticle. Lipid coated nanoparticles can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. In one embodiment, the lipid monolayer includes a PEGylated lipid.

The lipid bilayer which is used to prepare lipid coated nanoparticles can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the lipid coated nanoparticle in order to improve the lipid coated nanoparticle colloidal stability.

In certain diagnostic embodiments, various dyes or fluorescent (reporter) molecules can be included in the lipid coated nanoparticle cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed lipid coated nanoparticle can then be observed by, for example, confocal fluorescence for use in diagnostic applications. In addition, as discussed herein, plasmid DNA can be used as cargo in lipid coated nanoparticles such that the plasmid may express one or more fluorescent proteins such as fluorescent green protein or fluorescent red protein which may be used in diagnostic applications.

In various embodiments, the lipid coated nanoparticle may be used in a synergistic system where the lipid bilayer fusion or liposome fusion (i.e., on the porous particle core) is loaded and sealed with various cargo components with the pores (mesopores) of the particle core, thus lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final lipid coated nanoparticle

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or antiviral drugs such as anti-HBV or anti-HCV drugs) and other hydrophobic cargo such as fluorescent dyes.

In other embodiments, the cargo can be loaded into the pores (mesopores) of the porous particle cores to form the loaded lipid coated nanoparticle. In various embodiments, any conventional technology that js developed for liposome-based drug delivery, for example, targeted delivery using PEGylation, can be transferred and applied to the the lipid coated nanoparticles.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded lipid coated nanoparticle having a concentration exceed about 100 times as compared with the charged cargo components in a solution. In other embodiments, by varying the charge of the mesoporous particle and the lipid bilayer, positively charged cargo components can be readily loaded into lipid coated nanoparticles.

Once produced, the loaded lipid coated nanoparticles can have a cellular uptake for cargo delivery into a desirable site after administration. For example, the cargo-loaded lipid coated nanoparticles can be administered to a patient or subject and the lipid coated nanoparticle comprising a targeting peptide can bind to a target cell and be internalized or uptaken by the target cell, for example, a cancer cell in a subject or patient. Due to the internalization of the cargo-loaded lipid coated nanoparticles in the target cell, cargo components can then be delivered into the target cells. In certain embodiments the cargo is a small molecule, which can be delivered directly into the target cell for therapy.

### Exemplary Method

### Materials

All chemicals and reagents were used as received. Ammonium hydroxide (NH₄OH, 28-30%), 3-aminopropyltriethoxysilane (98%, APTES), ammonium nitrate (NH₄NO₃), benzyldimethylhexadecylammonium chloride (BDHAC), n-cetyltrimethylammonium bromide (CTAB), N,N-dimethyl formamide (DMF), dimethyl sulfoxide (DMSO), rhodamine B isothiocyanate (RITC), tetraethyl orthosilicate (TEOS), and Triton X-100 were purchased from Sigma-Aldrich (St. Louis, MO). Hydrochloric acid (36.5-38%, HCl) was purchased from EMD Chemicals (Gibbstown, NJ). Absolute (99.5%) and 95% ethanol were obtained from PHARMCO-AAPER (Brookfield, CT). 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine-N-[methoxy (polyethylene glycol)-2000](ammonium salt) (DOPE-PEG₂₀₀₀), 1,2-distearoyl-sn-glycero-3-phosphoethanolamine-N-[methoxy (polyethylene glycol)-2000](ammonium salt) (DSPE-PEG₂₀₀₀), 1,2-distearoyl-sn-glycero-3-phosphoethanolamine-N-[amino (polyethylene glycol)-2000] (DSPE-PEG₂₀₀₀—NH₂)phospholipids and cholesterol (Chol, ovine wool, >98%) were purchased from Avanti Polar Lipids (Birmingham, AL). Hoechst 33342, Traut's reagent, and maleimide-activated NeutrAvidin protein were obtained from Thermo Scientific (Rockford, IL). Alexa Fluor®488 phalloidin and CellTracker™ green CMFDA dye were purchased from Life Technologies (Eugene, OR). Heat inactivated fetal bovine serum (FBS), 10× phosphate buffered saline (PBS), 1× trypsin-EDTA solution, and penicillin streptomycin (PS) were purchased from Gibco (Logan, UT). Dulbecco's Modification of Eagle's Medium with 4.5 g/L glucose, L-glutamine and sodium pyruvate (DMEM) and RPMI-1640 medium were obtained from CORNING cellgro (Manassas, VA). Doxorubicin was purchased from LC Laboratories (Woburn, MA). Anti-EGFR antibody [EGFR1](Biotin) (ab24293) was purchased from Abcam (Cambridge, MA).

Synthesis of mMSNs composed of hexagonally arranged cylindrical pores (2.8 nm pore size). To prepare monosized dye-labeled mMSNs (about 95 nm in diameter, about 130 nm in hydrodynamic size in D.I. water), 3 mg of RITC was dissolved in 2 mL of DMF followed by addition of 1.5 μL APTES Townson et al., 2013). The synthesis conditions of mMSNs are based on reported literature (Lin and Haynes, 2011). The RITC-APTES solution was incubated at room temperature for at least 1 hour. Next, 290 mg of CTAB was dissolved in 150 mL of 0.51 M ammonium hydroxide solution in a 250 ml beaker, sealed with parafilm (Neenah, WI), and placed in a mineral oil bath at 50° C. After continuously stirring for 1 hour, 3 mL of 0.88 M TEOS solution (prepared in ethanol) and 1 mL of RITC-APTES solution were combined and added immediately to the surfactant solution. After another 1 hour of continuous stirring, the particle solution was stored at 50° C. for about 18 hours under static conditions. Next, solution was passed through a 1.0 μm Acrodisc 25 mm syringe filter (PALL Life Sciences, Ann Arbor, MI) followed by a hydrothermal treatment at 70° C. for 24 hours. Followed procedure for CTAB removal was as described in literature (Lin et al., 2011). Briefly, mMSNs were transferred to 75 mM ammonium nitrate solution (prepared in ethanol) then placed in an oil bath at 60° C. for 1 hour with reflux and stirring. The mMSNs were then washed in 95% ethanol and transferred to 12 mM HCl ethanolic solution and heated at 60° C. for 2 hours with reflux and stirring. Lastly, mMSNs were washed in 95% ethanol, then 99.5% ethanol, and stored in 99.5% ethanol.

Synthesis of spherical mMSNs with isotropic pores (2.5 nm pore size). To prepare monosized spherical mMSNs composed of isotropic mesopores, the same procedure described above for synthesis of mMSNs with hexagonally arranged pore structure was used. However, cationic surfactant BDHAC was substituted for CTAB as the template. The 3-dimensional isotropic pore arrangement is due to a larger micelle packing parameter of BDHAC, compared to CTAB surfactant (Chen et al., 2013).

Synthesis of dendrimer-like mMSNs composed of large pores (5 nm and 9 nm pore size). The large pore mMSNs were synthesized by a published biphase method (Bayu et al., 2009; Wang et al., 2012; Shen et al., 2014). Syntheses of 5 nm and 9 nm pore mMSNs are based on a modified condition reported by Zhao et al. (2014). For preparation of dendritic 5 nm pore mMSNs, 0.18 g of TEA was dissolved in 36 mL of DI water and 24 mL of 25 w % CTAC in a 100 mL round bottom flask. The surfactant solution was stirred at 150 rpm and heated at 50° C. in an oil bath. After 1 hour, 20 ml of 20 v/v % TEOS (in cyclohexane) was added to the CTAC-TEA aqueous solution. After 12 hours, the particle solution was washed with DI water twice by centrifugation. Further surfactant removal achieved by following the previously described conditions used in preparation of small pore mMSNs. For synthesis of 9 nm pore mMSNs, we adjusted the stirring rate and organic phase concentration to 300 rpm and 10 v/v % TEOS, respectively. All other steps were identical.

Synthesis of rod-shaped mMSNs with hexagonally arranged cylindrical pores (2.8 nm pore size). The shape of mMSNs can be simply tuned to rod-like morphology by altering the CTAB concentration, stirring rate, and ammonia concentration (Huang et al., 2011; Uy et al., 2011). Briefly, 0.5 g CTAB was dissolved in 150 ml of 0.22 M ammonium hydroxide solution at 25° C. under continuous stirring (300 rpm). Next, of 1 mL TEOS was added (drop wise) to the surfactant solution with stirring. After 1 hour, the particle solution was aged under static conditions for 24 hours, then subsequently transferred to a sealed container and heated to 70° C. for 24 hours. The removal of surfactant was followed the same procedures described previously.

Liposome preparation. Lipids and cholesterol ordered from Avanti Polar Lipids were presolubilized in chloroform at 25 mg/mL and were stored at −20° C. To prepare liposomes, lipids were mixed at different mol % ratios including (54/44/2) for DOPC/Chol/DOPE-PEG₂₀₀₀ and DSPC/Chol/DSPE-PEG₂₀₀₀, and (49/49/2) for DSPC/Chol/DSPE-PEG₂₀₀₀—NH₂. Lipid films were prepared by drying lipid mixtures (in chloroform) under high vacuum to remove the organic solvent. Then the lipid film was hydrated in 0.5× PBS and bath sonicated for 30 minutes to obtain a liposome solution. Finally, the liposome solution was further passed through a 0.05 μm polycarbonate filter membrane (minimum 21 passes) using a mini-extruder to produce uniform and unilamellar vesicles with hydrodynamic diameters less than 100 nm.

Lipid coated nanoparticle preparation. To form lipid coated nanoparticles, mMSNs are transferred to D.I. water at 1 mg/mL concentration by centrifugation (15,000 g, 10 minutes) and added to liposome solution in 0.5× PBS (1:1 v/v and 1:2 w/w ratios). The mixture was bath sonicated about 10 seconds and non-fused liposomes were removed by centrifugation (15,000 g, 10 minutes). Pelleted lipid coated nanoparticles were redispersed in 1×PBS via bath sonication, this step is repeated twice.

Anti-EGFR lipid coated nanoparticle preparation. First, DSPC/Chol/DSPE-PEG-NH₂ liposomes were prepared according to the method described previously. Next, a ratio (2:1, w: w) of DSPC/Chol/DSPE-PEG₂₀₀₀—NH₂ liposomes to bare RITC labeled mMSN were combined in a conical tube at room temperature for 30 minutes. The excess liposomes were removed by centrifugation (15,000 g, 10 minutes). The pelleted lipid coated nanoparticles were redispersed in 1 mL of PBS with bath sonication. To convert the surface-NH₂ to —SH groups, 50 μL of freshly prepared Traut's reagent (250 mM in PBS) was added to the lipid coated nanoparticles. After 1 hour, the particles were centrifuged, and the supernatant was removed. The particles were again redispersed in 1 mL of PBS. Then, 0.15 mg of maleimide-activated NeutrAvidin protein was added to 0.25 mL of thiolated lipid coated nanoparticles and incubated at room temperature for 12 hours. The NeutrAvidin conjugated lipid coated nanoparticles were washed with PBS via centrifugation and suspended in 0.25 mL of PBS. Then, 50 μL of biotinylated EGFR antibody (0.1 mg/mL) was mixed with 50 μL of NeutrAvidin conjugated lipid coated nanoparticles for at least 30 minutes. Finally, the antibody conjugated lipid coated nanoparticles were pelleted and redispersed in 100 μL PBS for in vitro targeting experiments.

In vitro red blood cell compatibility. Whole human blood was acquired from healthy donors with informed consent and stabilized in K2EDTA tubes (BD Biosciences). hRBCs were purified following reported procedure (Liao et al., 2010), then incubated with either bare mMSNs or lipid coated nanoparticles (25, 50, 100, 200, and 400 μg/mL) at 37° C. After 3 hours of exposure, samples were centrifuged at 300 g for 3 minutes, then 100 μL of supernatant from each sample was transferred to a 96-well plate. Hemoglobin absorbance was measured using a BioTek microplate reader (Winooski, VT) at 541 nm. The percent hemolysis of each sample was quantified using a reported equation (Liao et al., 2011).

Cell culture and nanoparticle nonspecific binding/uptake. Human endothelial cells, EA.hy926 (CRL-2922) were purchased from American Type Culture Center (ATCC, Manassas, VA). We seeded 5×10⁵ EA.hy926 cells in 6-well plates with 2 mL of DMEM+10% FBS and 1% PS at 37° C. in 5% CO2 humidified atmosphere. After 24 hours, the media was removed and replaced with 2 mL of fresh complete media supplemented with 20 μg/mL of bare mMSNs or lipid coated nanoparticles for 4 hours at 37° C. under 5% CO₂. After nanoparticle incubation, the media was removed and the cells were gently washed twice with PBS. For imaging purposes, the nanoparticle treated cells were fixed in 3.7% formaldehyde (in PBS) at room temperature for 10 minutes, washed with PBS, then treated with 0.1% Triton X-100 for another 10 minutes. The fixed cells were washed with PBS and stored in 1 mL of PBS. The cell nuclei and F-actin were stained with 1 mL of Hoechst 33342 (3.2 μM in PBS) and 0.5 mL of Alexa Fluor®488 phalloidin (20 nM in PBS) for 20 minutes, respectively. After staining, the cells were washed with PBS twice and stored in PBS prior to fluorescence microscope imaging. For preparation of flow cytometry samples, the control and nanoparticle treated cells were removed from plate bottom using Trypsin-EDTA (0.25%). The suspended cells were centrifuged, washed with PBS, and suspended in PBS for flow cytometry measurements.

Cell-nanoparticle interactions in ex ovo avian embryos. Ex ovo avian embryos were handled according to published methods (Leong et al., 2010), with all experiments conducted following an institutional approval protocol (11-100652-T-HSC). This method included incubation of fertilized eggs (purchased from East Mountain Hatchery-Edgewood, NM) in a GQF 1500 Digital Professional egg incubator (Savannah, GA) for 3-4 days. Following initial in ovo incubation, embryos were removed from shells by cracking into 100 mL polystyrene weigh boats (VWR, Radnor, PA). Ex ovo embryos were then covered and incubated (about 39° C.) with constant humidity (about 70%). For nanoparticle injections, about 50 μg (at 1 mg/mL) of bare mMSNs or lipid coated nanoparticles in PBS were injected into secondary or tertiary veins of the CAM via pulled glass capillary needles. CAM vasculature and fluorescent nanoparticles were imaged using a customized avian embryo chamber (humidified) and a Zeiss AxioExaminer upright microscope modified with a heated stage. High speed videos were acquired on the same microscope using a Hamamatsu Orca Flash 4.0 camera.

Post-circulation size and stability analyses. All animal care and experimental protocols were in accordance with the National Institutes of Health and University of New Mexico School of Medicine guidelines. Ten—to twelve-week-old female BALB/c mice (Charles River Laboratories, Wilmington, MA) were administered dose of RITC-labeled lipid coated nanoparticles (10 mg/mL) in 150 μL PBS via tail vein injection. After 10 minutes of circulation, mice were euthanized and blood was drawn by cardiac puncture. Whole blood was stabilized in K2EDTA microtainers (BD Biosciences) prior to analysis. Ex ovo avian embryos were administered dose of RITC-labeled lipid coated nanoparticles (1 mg/mL) in 50 μL PBS via secondary or tertiary veins of the CAM. After 10 minutes of circulation, blood was drawn via pulled glass capillary needles and analyzed immediately. Whole blood cells and lipid coated nanoparticle fluorescence in both mouse and avian samples were imaged on a glass slide with Zeiss AxioExaminer fixed stage microscope (Gottingen, Germany). To separate lipid coated nanoparticles from whole blood, samples were centrifuged at low speed to remove blood cells, supernatant fraction was transferred to a fresh tube then centrifuged at 15,000 g for 10 minutes. The pellets were washed (15,000g for 10 min) twice in PBS, then lipid coated nanoparticle size was analyzed on Malvern Zetasizer Nano-ZS equipment.

In vitro targeting. The pro-B-lymphocyte cell lines, Ba/F3 and Ba/F3+EGFR (Li et al., 1995) were a kind gift from Professor David F. Stern, Yale University. The Ba/F3 and Ba/F3+EGFR cells were suspended in RPMI 1640 supplemented with 10% FBS media at a concentration of about 1×10⁶ cells/mL. Then one mL of cells was incubated with anti-EGFR lipid coated nanoparticles at 5 μg/mL for 1 hour at 37° C. under 5% CO₂. The cell nuclei and membrane were stained by 1 μL of Hoechst 33342 (1.6 mM in DI) and 2 μL of CellTracker™ green CMFDA dye (2.7 mM in DMSO) for 10 minutes. The nanoparticle-treated cells were pelleted using a benchtop centrifuge, washed with PBS twice, and dispersed in PBS. The live cells were imaged on a glass slide using the Zeiss AxioExaminer upright microscope. To further examine the specificity of targeted lipid coated nanoparticles, the binding of particles was determined by a fluorescence shift measured by a Becton-Dickinson FACScalibur flow cytometer.

In vivo single cell targeting in ex ovo chicken embryos. First, about 1×10⁶ of BAF+EGFR cells were suspended in 1 mL PBS and incubated with 2 μL of CellTracker™ green CMFDA dye for 10 minutes at 37° C. The stained cells were centrifuged, washed, and suspended in 500 μL of PBS. Next, 50 μL of cell solution was administered to ex ovo avian embryos via the previously described procedure. After 30 minutes cell circulation, the anti-EGFR lipid coated nanoparticles (100 μL, 0.2 mg/mL) were injected into embryos intravenously. The binding and internalization of targeted lipid coated nanoparticles to cancer cells was imaged at different time points using the Zeiss AxioExaminer upright microscope.

Characterization. TEM images were acquired on a JEOL 2010 (Tokyo, Japan) equipped with a Gatan Orius digital camera system (Warrendale, PA) under a 200 kV voltage. The cryo-TEM samples were prepared using an FEI Vitrobot Mark IV (Eindhoven, Netherlands) on Quantifoil® R1.2/1.3 holey carbon grids (sample volume of 4 μL, a blot force of 1, and blot and drain times of 4 and 0.5 seconds, respectively). Imaging was taken with a JEOL 2010 TEM at 200 kV using a Gatan model 626 cryo stage. Nitrogen adsorption-desorption isotherms of mMSNs were obtained from on a Micromeritics ASAP 2020 (Norcross, GA) at 77 K. Samples were degassed at 120° C. for 12 hours before measurements. The surface area and pore size was calculated following the Brunauer-Emmet-Teller (BET) equation in the range of P/Po from 0.05 to 0.1 and standard Barrett-Joyer-Halenda (BJH) method. Flow cytometry data were performed on a Becton-Dickinson FACScalibur flow cytometer (Sunnyvale, CA). The raw data obtained from the flow cytometer was processed with FlowJo software (Tree Star, Inc. Ashland, OR). Hydrodynamic size and zeta potential data were acquired on a Malvern Zetasizer Nano-ZS equipped with a He-Ne laser (633 nm) and Non-Invasive Backscatter optics (NIBS). All samples for DLS measurements were suspended in various media (DI, PBS, and DMEM+10% FBS) at 1 mg/mL. Measurements were acquired at 25° C. or 37° C. DLS measurements for each sample were obtained in triplicate. The Z-average diameter was used for all reported hydrodynamic size measurements. The zeta potential of each sample was measured in 1×PBS using monomodal analysis. All reported values correspond to the average of at least three independent samples. The fluorescence images were captured with a Zeiss AxioExaminer fixed stage microscope (Gottingen, Germany). Additional information-Calculation for Examples

Calculations to identify optimal liposome to mMSN surface area ratio. To estimate the number of particles in solution (n), a spherical model was employed to calculate mMSN exterior surface area (SA) and volume (V_{mMSN}) from diameter (D) obtained from Z-average DLS measurements, pore volume (Vₚₒᵣₑ) measurements from nitrogen adsorption-desorption isotherms (0.73 cm³/g), and a mesoporous silica density (p) of 2 g/cm³.

The equations below were used to estimate the number of particles in solution per unit concentration.

$$S {A}_{m M S N} = 4 π * {( D / 2 )}^{2}$$

$${V}_{m M S N} = 4 / 3 * π * {( {D}_{m M S N} / 2 )}^{3}$$

$${n}_{m M S N} = \frac{( m / ρ ) + ( m * {V}_{p o r e} )}{{V}_{m M S N}}$$

Next we determined the theoretical inner and outer surface areas (SAᵢₙₙₙₑᵣ and SAₒᵤₜₑᵣ) of an individual liposome using the diameter (D) obtained from Z-average DLS measurements of mMSNs and assuming lipid bilayer thickness (d) of 5.1 nm

$$S {A}_{inner} = 4 π * {( D / 2 )}^{2}$$

$$S {A}_{outer} = 4 π * {[ ( D / 2 ) + d ]}^{2}$$

$$S {A}_{l i p o s o m e} = S {A}_{inner} + S {A}_{outer}$$

To find the number of component molecules needed to occupy the total theoretical liposome surface area we use the mass used (m) and assume 0.7 nm² to represent single lipid head group area [1] and 0.38 nm 2 for cholesterol group area. [2]

$${Moles}_{c o m ponent} = \frac{{m}_{c o m ponent}}{M {W}_{c o m ponent}}$$

$${Moles}_{l i p o s o m e} = {\sum }_{i = 1}^{n} {Moles}_{{i}_{c o m ponent}}$$

$$S {A}_{average component} = \frac{( 0.7 * {Σ}_{i = 1}^{n} {Moles}_{{i}_{c o m ponent}} + 0.38 * {Moles}_{c holesterol} )}{M o l e {s}_{l i p o s o m e}}$$

$${Molecules}_{n e e d e d} = \frac{( {n}_{m M S N} * S {A}_{L i p o s o m e} )}{S {A}_{average component}}$$

To find the optimal mass of lipid to a fixed mMSN amount, we use the total mass of the liposome components and convert the molecules needed to mass needed.

$$M {W}_{average liposomes} = \frac{{Σ}_{i = 1}^{n} [ {Moles}_{{i}_{c o m ponent}} * M {W}_{i} ]}{{Moles}_{liposome}}$$

$${m}_{liposomes} = {\sum }_{i = 1}^{n} {Concentation}_{{i}_{c o m ponent}} * {V}_{{i}_{c o m ponent}}$$

$${Moles}_{n e e d e d} = {Molecules}_{n e e d e d} / {N}_{A}$$

$${m}_{n e e d e d} = {Moles}_{n e e d e d} * M {W}_{average liposomes}$$

The calculated mass of fluorescent liposome (DSPC/Chol/DSPE-PEG₂₀₀₀/NBD-Chol-54/43/2/1 mol %) to mMSN (118.7 nm Z-average diameter) is 0.263 to 1. The experimental quantification of mass of fluorescent labeled liposome to mMSN is 0.276 to 1, as measured from fluorescence intensity of unbound liposomes in the supernatant following centrifugation of the lipid coated nanoparticles compared to a standard curve generated from known fluorescent liposome concentration. The calculated and experimental values are within 4.7% of each other, which is supportive of our method of surface area ratio calculations.

### Lipid Coated Nanoparticle Example

### Design, Synthesis, and Characterization of Nanocarrier Silica-Supported Multilamellar Lipid Bilayer (Lipid Coated Nanoparticles).

Lipid coated nanoparticles are composed of a nanoporous nanoparticle core that supports a lipid bilayer, which is further conjugated with targeting peptides and polyethylene glycol (PEG). Through engineering the pore size and surface chemistry, as well as the degree of condensation of the nanoporous particle core (which serves as a reservoir for arbitrary multicomponent cargos), the cargo loading and release characteristics we tailored to achieve optimized pharmacokinetics and biodistribution of therapeutic agents via in vitro and in vivo studies. The biophysical and biochemical properties of the supported lipid bilayer, such as fluidity and peptide types and concentrations, are refined through iterative studies to maximize binding to and internalization within target cells. The outer lipid coated nanoparticle surfaces are functionalized with octa-arginine (R8) peptide, to induce cellular uptake of the lipid coated nanoparticle through macropinocytosis. In addition, Toll-like receptor (TLR) agonists including Monophosphoryl lipid A (MPLA), a derivative of the lipopolysaccharide layer of Salmonella minnesota recognized by TLR-4, and Flagellin, a protein monomer that contains highly conserved regions recognized by TLR-5, among numerous others as described hereinabove. The innermost lipid bilayer will be functionalized with H5WYG, an endosomolytic peptide that promotes endosomal escape to allow for delivery of cargo components to the cytoplasm of the target cell. Cell culture studies of targeted lipid coated nanoparticle selectivity and fluorescently labeled cargo delivery.

Flow cytometry is employed to determine the specific affinity of lipid coated nanoparticles modified with various densities of TLR agonists to cultured peripheral blood mononuclear cell (PBMC) derived dendritic cells. The full length viral proteins incorporated into the lipid coated nanoparticle will be fluorescently labeled. In addition, the proteins encapsulated in the core will be ubiquitinylated to facilitate rapid proteasome degradation. The degree of R8/TLR induced lipid coated nanoparticle internalization and the intracellular fate of internalized cargo will be assessed using fluorescence confocal microscopy. As described above, the fluidity of the lipid coated nanoparticles is modified and the degree of PEG present on the nanocarrier surfaces altered to modulate targeting efficacy, maximize the ratio of internalized versus surface-bound nanoparticles, and increase colloidal stability in the presence of serum proteins and physiological salt concentrations. In vitro toxicology studies are performed by assessing the degree of oxidative stress induced in target and control cells by lipid coated nanoparticles.

### Targeting of APCs to Initiate Adaptive Immune Response to Nipah Virus-Specific Proteins in an Animal Model.

Animals are inoculated intramuscularly with multiple Lipid coated nanoparticle variations and compared to Nipah viral proteins alone. The animals are immunized two times at two-week intervals, and blood will be collected from animals two weeks after each inoculation via intraocular bleed. Activated T cells are isolated from whole blood and total T cell population will be compared to negative control to determine whether lipid coated nanoparticles effectively stimulate T cell proliferation. In addition, titers of the resulting anti-Nipah viral protein antibodies elicited are assayed by indirect ELISA. Following immunization, animals are challenged with live Nipah virus (BSL-4 in Texas). Animals are sacrificed day X post infection, and tissue including brain, lung, mediastinal lymph nodes, spleen, and kidney will be harvested for immunohistochemistry analysis using antisera to Nipah virus. Lastly, to examine the therapeutic potential of lipid coated nanoparticles, animals are infected with Nipah virus and at different time points after exposure, will be inoculated with Lipid coated nanoparticles. Blood will be collected from the animals at multiple time points and viral load will be assessed by indirect ELISA.

### Example 3

Cargo loading and release kinetics. Model drug loading was achieved by adding 1% volume YO-PRO®-1 (1 mM in DMSO) to mMSNs (1 mg/mL in H₂O) and stored for 12 hours at 4° C. After loading, targeted lipid coated nanoparticles were prepared using method described earlier in Anti-EGFR targeted lipid coated nanoparticle preparation. We observed a color change in the pelleted YO-PRO®-1 loaded lipid coated nanoparticles and did not observe any color in the supernatant during lipid coated nanoparticle assembly. The interaction between YO-PRO®-1 and mMSNs may largely be driven by electrostatics, since YO-PRO®-1 carries a positive charge. Moreover, YO-PRO®-1 is membrane impermeable, therefore, it should remain encapsulated by the SLB of the lipid coated nanoparticle until it is broken down in the intracellular environment. To quantify YO-PRO®-1 loading, lipid coated nanoparticles were pelleted by centrifugation and resuspended in DMSO with bath sonication, this step was repeated twice. Supernatants were pooled and concentration was determined using a microplate reader fluorescence measurement at 480/510 nm. A mean 25% loading efficiency of YO-PRO®-1 was calculated for lipid coated nanoparticles used in the model drug delivery experiments in vitro and ex ovo. To load and quantify gemcitabine (GEM), 0.5 mg of Hexagonal mMSNs (m_{mMSN}) were suspended in 50 μL of GEM dissolved in DI water at 10 mg/mL (m_{GEM}=0.5 mg) and stored for 12 hours at 4° C. After drug loading, targeted lipid coated nanoparticles were prepared using method described earlier in Anti-EGFR targeted lipid coated nanoparticle preparation. At each step, supernatant was collected, pooled (V1=2.55 mL), and GEM loading was determined using a microplate reader absorbance measurement at 265 nm. A standard curve generated from a serial dilution of GEM in PBS (n=3) was used to calculate the concentration of GEM in the supernatant. To account for absorbance signal from non-GEM components in the supernatant, unloaded lipid coated nanoparticles were prepared simultaneously under identical conditions and measured at 265 nm. This absorbance value (Abs_{control}) was subtracted from the value obtained from supernatant containing GEM (AbS_{GEM}) prior to calculation of GEM concentration based on standard curve [C₁=(Abs-0.0507)/7.7115]. For example, we used (m_{mMSN}=0.5 mg), and (M_{GEM}=0.5 mg) and we obtained (Abs_{GEM}=2.51) and (Abs_{control}=1.18). To solve for the amount loaded [AbS_{GEM}-AbS_{control}]=1.33, then GEM amount in the supernatant can be calculated by [C₁=(1.33-0.0507)/7.7115]=0.17 mg/mL. The total volume of the pooled supernatant is used to calculate the amount of GEM in the supernatant (m₁=C₁*v₁) or (m₁=0.17 mg/ml*2.55 mL)=0.43 mg. The supernatant amount (m₁) was then subtracted from the starting GEM amount (m_{GEM}) to estimate the total amount loaded into lipid coated nanoparticles [m_{loaded}=m₀-m₁] or (0.5 mg-0.43 mg)=0.07 mg. To estimate the loading capacity as a percentage of weight we use the formula [(m_{loaded}/M_{mMSN})*100%]or (0.07 mg/0.5 mg)*100%=14% (w/w). This experiment was repeated 4 times with different Hexagonal mMSN preparations and we determined the average GEM loading capacity of lipid coated nanoparticle=15.25%±1.6% (mean±SD). While the loading percentage of our lipid coated nanoparticles is lower than what was reported by Dr. Nel's group, the present loading conditions contain half the amount of GEM that was described by the Meng et al. (2015). Since GEM is neutral at physiological pH, and mMSNs are negatively charged, we do not suspect an electrostatic interaction to play a significant role in loading, instead suspect the GEM and mMSNs will reach an equilibrium state where the small molecule drug will occupy the high internal space of the pores and will then be encapsulated with the addition of the lipid bilayer in lipid coated nanoparticle assembly. A 3.5-5 kD MWCO Float-A-Lyzer was used to evaluate GEM release kinetics in either PBS (pH 7.4) or citrate buffer (pH 5.0). GEM was encapsulated into lipid coated nanoparticles as described above, then lipid coated nanoparticles were loaded into Float-A-lyzers and sealed in 50 ml conical tubes containing either PBS or citrate buffer, and stored at 37° C. while stirring. 0.5 mL of dialysate was removed for 265 nm absorbance analysis on BioTek microplate reader at multiple time points, then added 0.5 mL of fresh dialysate solution to the conical tube. To assess lipid coated nanoparticle size at 24 and 72 hours a sample removed from the Float-α-Lyzer, and the hydrodynamic size measured on Malvern Zetasizer Nano ZS, then it was placed back inside the Float-α-Lyzer and stored at 37° C. while stirring. Consistent with findings reported by Meng et al. (2015), there was no evidence of drug precipitation and the effective release of GEM was determined by cell viability analysis. In addition, the loaded and targeted lipid coated nanoparticles maintained monodispersity.

Targeted lipid coated nanoparticle GEM delivery and cytotoxicity assessment. About 1.5×10⁵ cells/mL of REH and REH+EGFR cell lines were incubated with either 0, 1, 5, 10, 25, or 50 μg/mL of GEM loaded (about 15% w/w) anti-EGFR targeted lipid coated nanoparticles in complete medium for 1 hour at 37° C. Cells were centrifuged (500 g, 3 minutes) and washed twice in complete media and transferred to a white 96-well plate for 24 hours at 37° C. In comparison, about 1.5×10⁵ cells/mL of REH and REH+EGFR cell lines were incubated with either 0, 0.6, 3, 6, 15, or 30 μM of free GEM, the equivalent doses based on 15% (w/w) GEM loading into lipid coated nanoparticles, under identical experimental conditions. Cell viability was assessed by CellTiter-Glo® 2.0 Assay as measured by BioTek microplate reader. The cell viability was calculated as a percentage of non-lipid coated nanoparticle treated sample.

In vitro internalization and cargo release assay. REH+EGFR cells were suspended in RPMI 1640 supplemented with 10% FBS media at a concentration of 5×10⁵ cells/mL. Then one mL of cells was incubated with YO-PRO®-1 loaded, RITC-labelled anti-EGFR lipid coated nanoparticles at 10 μg/mL for 60 minutes at 37° C., washed twice in media to remove unbound lipid coated nanoparticles, and incubated for 1, 8, 16, and 24 hours respectively at 37° C. under 5% CO₂. The lipid coated nanoparticle-treated cells were pelleted using a benchtop centrifuge, at each time point, and resuspended in an acid wash solution (0.2 M acetic acid, 0.5 M NaCl, pH 2.8) and incubated on ice for 5 minutes. Cells were then washed twice with PBS by centrifugation and lipid coated nanoparticle internalization was assessed by a red fluorescence shift and cargo release was assessed by a green fluorescence shift as measured by a BD Accuri™ C6 flow cytometer. Additionally, live cells were imaged on a glass slide using the Leica DMI3000 B inverted microscope.

Calculations to Identify Optimal Liposome to mMSN Surface Area Ratio.

To estimate the number of particles in solution (n), a shape applicable model was employed to calculate mMSN exterior surface area (SA) and volume (V_{mMSN}) from dimensional measurements obtained from TEM image analysis (n=50), pore volume (Vₚₒᵣₑ) measurements from nitrogen adsorption-desorption isotherms, a mesoporous silica density (p) of 2 g/cm³, and a sample mass (m). The equations below were used to estimate the number of particles in solution per unit concentration (mg/ml) and the external particle surface areas (nm²) used in determination of the lipid silica surface area ratio.

Monosized lipid coated nanoparticles prepared from MSNs provide an advantageous approach to treatment of a large variety of disease states and conditions, especially where targeted drug delivery provides an advantageous approach to such treatment by increasing the therapeutic effect and/or reducing side effects associated with the use of prior art formulations and methods. In addition, in certain embodiments, lipid coated nanoparticles exhibit enhanced colloidal and/or storage stability in solution.

### Exemplary Embodiments

In one embodiment, a multi-layer coated nanoparticle wherein the nanoparticle comprises a first coat comprising a polyamine or a cationic lipid and a second coat comprising an anionic lipid layer is provided. In one embodiment, the first coat comprises a polyamine. In one embodiment, the nanoparticle is a mesoporous silica nanoparticle. In one embodiment, the nanoparticle further comprises a cargo. In one embodiment, the cargo comprises one or more prophylactic or therapeutic agents. In one embodiment, the cargo comprises RNA, e.g., wherein the RNA comprises siRNA. In one embodiment, the cargo is negatively charged. In one embodiment, the first coat is between the nanoparticle and the second coat. In one embodiment, the nanoparticle further comprises a targeting ligand. In one embodiment, the anionic lipid layer is a bilayer. In one embodiment the polyamine has a molecular weight of about ot less than 20 KDa, e.g., from about 15 kDa to about 20 kDa. In one embodiment, the polyamine has a molecular weight of about or less than 10 KDa, e.g., from about 5 kDa to about 10 kDa. In one embodiment, the polyamine has a molecular weight of about or less than 5 KDa, e.g., from about 1 kDa to about 5 kDa. In one embodiment, the polyamine has a molecular weight of about 2 KDa. In one embodiment, the polyamine has a molecular weight of about 1 kDa to about 4 kDa. In one embodiment, th polyamine comprises polyethylenimine (PEI), polyacrylamide (PAM), poly-lysine, or spermine, spermidine, diamine. In one embodiment, the polyamine is a linear polyamine. In one embodiment, the polyamine is a branched polyamine. In one embodiment, the polyamine is a linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, or PEI-mannose-dextrose. In one embodiment, the nanoparticle has a diameter of about 100 nm to 400 nm. In one embodiment, the nanoparticle comprises pores. In one embodiment, the pores have a diameter of about 1 to 10 nm. In one embodiment, the weight ratio of polyamine to nanoparticle is 0.05 to 10. In one embodiment, the weight ratio of polyamine to nanoparticle is 0.05 to 2. In one embodiment, the weight ratio of polyamine to nanoparticle is 0.1 to 0.6.

In one embodiment, a method of making the nanoparticleis provided, comprising: combining an amount of nanoparticles and a composition comprising an amount of a polyamine so as to form a polyamine coat on the nanoparticles; and combining the polyamine coated nanoparticles and a composition comprising an amount of anionic lipid so as to form an anionic lipid coat on the polyamine coated nanoparticles. In one embodiment, the method further comprises combining one or more prophylactic or therapeutic agents with the nanoparticles and the polyamine. In one embodiment, the method further comprises combining one or more prophylactic or therapeutic agents with the nanoparticles before the nanoparticles are combined with the polyamine. In one embodiment, the method further comprises combining one or more prophylactic or therapeutic agents with the polyamine coated nanoparticles before the polyamine coated nanoparticles are combined with the anionic lipid. In one embodiment, the anionic lipid comprises a targeting moiety.

Also provided is a method of using the nanoparticle, e.g., by administering to a mammal a composition comprising the nanoparticle.

In one embodiment, the nanoparticles are monosized. In one embodiment, the solution comprises buffered saline. In one embodiment, the population of lipid coated nanoparticles has a polydispersity index of less than about 0.1. In one embodiment, said nanoparticles are spheroidal, ellipsoidal, triangular, rectangular polygonal or hexagonal prisms. In one embodiment, said liposomes have an internal surface area larger than an external surface area of said nanoparticles. In one embodiment, said lipid bilayer has a lipid transition temperature (Tm) which is greater than the temperature at which said population of lipid coated nanoparticles will be stored or used. In one embodiment, said lipid bilayer comprises more than about 50 mole percent an anionic phospholipid.

In one embodiment, said lipid bilayer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof. The population of lipid coated nanoparticles according to any one of claims 1-16 wherein said lipid bilayer comprises 1,2-distearoyl-sn-glycero-3-phosphocholine (DSPC), 1,2-dioleoyl-sn-glycero-3-phosphoethanolamine (DOPE), or a mixture thereof. The population of lipid coated nanoparticles according to any one of claims 1-17 wherein said lipid bilayer comprises cholesterol. The population of lipid coated nanoparticles according to any one of claims 1-18 wherein said lipid bilayer comprises about 0.1 mole percent to about 25 mole percent of at least one lipid comprising a functional group to which a functional moiety may be covalently attached. The population of lipid coated nanoparticles according to claim 19 wherein said lipid comprising a function group is a PEG-containing lipid.

In one embodiment, said PEG-containing lipid is selected from the group consisting of 1,2-dioleoyl-sn-glycero-3-phosphoethanolamine-N-[methoxy (polyethylene glycol)](ammonium salt) (DOPE-PEG), 1,2-distearoyl-sn-glycero-3-phosphoethanolamine-N-[methoxy (polyethylene glycol)](ammonium salt) (DSPE-PEG), 1,2-distearoyl-sn-glycero-3-phosphoethanolamine-N-[amino (polyethylene glycol)](DSPE-PEG-NH₂), or a mixture thereof.

In one embodiment, said lipid coated nanoparticles comprise at least one component selected from the group consisting of: a cell targeting species; a fusogenic peptide; and a cargo, wherein said cargo is optionally conjugated to a nuclear localization sequence. In one embodiment, said lipid coated nanoparticles comprise a cell targeting species. In one embodiment, said cell targeting species is a peptide, an antibody, an affibody or a small molecule moiety which binds to a cell. In one embodiment, said lipid coated nanoparticles comprise a fusogenic peptide. In one embodiment, said fusogenic peptide is H5WYG peptide, 8 mer polyarginine, RALA peptide, KALA peptide, GALA peptide, INF7 peptide, or a mixture thereof. In one embodiment, said lipid coated nanoparticles comprise a cargo. In one embodiment, said cargo is an anti-cancer agent, anti-viral agent, an antibiotic, an antifungal agent, a polynucleotide, a peptide, a protein, an imaging agent, or a mixture thereof. In one embodiment, said polynucleotide comprises encapsulated DNA, double stranded linear DNA, a plasmid DNA, small interfering RNA, small hairpin RNA, microRNA, or mixtures thereof. A storage stable composition comprising a population of lipid coated nanoparticles, in one embodiment, in an aqueous solution. In one embodiment, said aqueous solution comprises a saline solution. A pharmaceutical composition comprising a population of lipid coated nanoparticles, in one embodiment, and a pharmaceutically acceptable excipient.

In one embodiment, the nanoparticles comprise silica. In one embodiment, the nanoparticles are mesoporous. In one embodiment, the nanoparticles are monosized. And said lipid bilayer comprises more than about 50 mole percent of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000](18:1 PEG-2000 PE), 1,2-dipalmitoyl-sn-glycero-3-phosphoethanolamine-N-[methoxy (polyethylene glycol)-2000](16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid bilayer comprises 1,2-distearoyl-sn-glycero-3-phosphocholine (DSPC), 1,2-dioleoyl-sn-glycero-3-phosphoethanolamine (DOPE), or a mixture thereof; or wherein said lipid bilayer comprises cholesterol. In one embodiment, said lipid bilayer comprises about 0.1 mole percent to about 25 mole percent of at least one lipid comprising a functional group to which a functional moiety may be covalently attached.

In one embodiment, said lipid comprising a function group is a PEG-containing lipid, optionally wherein said PEG-containing lipid is selected from the group consisting of 1,2-dioleoyl-sn-glycero-3-phosphoethanolamine-N-[methoxy (polyethylene glycol)](ammonium salt) (DOPE-PEG), 1,2-distearoyl-sn-glycero-3-phosphoethanolamine-N-[methoxy (polyethylene glycol)](ammonium salt) (DSPE-PEG), 1,2-distearoyl-sn-glycero-3-phosphoethanolamine-N-[amino (polyethylene glycol)](DSPE-PEG-NH₂), or a mixture thereof. In one embodiment, said lipid coated nanoparticles comprise at least one component selected from the group consisting of: a cell targeting species; a fusogenic peptide; and a cargo, wherein said cargo is optionally conjugated to a nuclear localization sequence. In one embodiment, said cell targeting species is a peptide, an antibody, an affibody or a small molecule moiety which binds to a cell. In one embodiment, said lipid coated nanoparticles comprise a fusogenic peptide, and optionally wherein said fusogenic peptide is H5WYG peptide, 8 mer polyarginine, RALA peptide, KALA peptide, GALA peptide, INF7 peptide, or a mixture thereof, said cargo is an anti-cancer agent, anti-viral agent, an antibiotic, an antifungal agent, a polynucleotide, a peptide, a protein, an imaging agent, or a mixture thereof. In one embodiment, said polynucleotide comprises encapsulated DNA, double stranded linear DNA, a plasmid DNA, small interfering RNA, small hairpin RNA, microRNA, or mixtures thereof.

The multilamellar nanoparticle comprises a nanoporous silica or metal oxide core and at least an inner polyamine and an outer lipid bilayer and optionally an inner aqueous layer and/or an outer aqueous layer, said inner aqueous layer separating said core from said inner polyamine layer and said outer aqueous layer separating said inner polyamine layer from said outer lipid bilayer said outer lipid bilayer optionally comprising: at least one targeting ligand, e.g., a Toll-like receptor (TLR) agonist; a fusogenic peptide; and optionally at least one cell targeting species which selectively binds to a target, e.g., on antigen presenting cells (APCs).

A pharmaceutical composition comprising a population of the lipid coated nanoparticles in combination with a pharmaceutically acceptable carrier, additive or excipient is also provided. In one embodiment, the composition further comprises a drug, reporter or adjuvant in combination with said population of lipid coated nanoparticles. A vaccine comprising the composition optionally in combination with an adjuvant, is further provided. A method of inducing an immunogenic response in a subject is provided, wherein a subject is administered an effective amount of the composition. A method inducing immunity to a microbial infection in a subject is also provided comprising administering at least once, an effective amount of the composition to a subject. In one embodiment, said composition is administered as a booster subsequent to a first administration of said composition.

In one embodiment, a multi-layer coated nanoparticle is provided wherein the nanoparticle comprises a first coat comprising a positively charged polymer or a positively charged lipid bilayer and a second coat comprising an anionic lipid bilayer, wherein the multi-layer nanoparticle optionally comprises one or more distinct cargo molecules, one or more distinct targeting moieties, or any combination thereof. In one embodiment, the positively charged polymer comprises a polyamine. In one embodiment, the positively charged polymer comprises polyethyleneimine (PEI), polyamidoamine (PAMAM), or poly(β-amino ester). In one embodiment, the positively charged lipd bilayer comprises one or more of dipalmitylphosphatidylcholine (DPPC), dioleoyl-3-trimethylammonium propatre (DOTAP), 1, 2-dioleoyloxy-3-dimethylamino propane (DODMA), or cholesterol. In one embodiment, the nanoparticle comprises a molar ratio of DPPC of 70 to 90 or 30 to 90. In one embodiment, the nanoparticle comprises a molar ratio of DPPC of 75 to 85. In one embodiment, the nanoparticle comprises a molar ratio DOTAP or DODMA of 5 to 15 or 5 to 50. In one embodiment, the nanoparticle comprises a molar ratio of DOTAP or DODMA of 7 to 12. In one embodiment, the nanoparticle comprises a molar ratio of cholesterol of 5 to 15 or 0 to 40. In one embodiment, the nanoparticle comprises a molar ratio of cholesterol of 7 to 12. In one embodiment, the positively charged lipd bilayer does not include polyethylene glycol (PEG). In one embodiment, the anionic lipid bilayer comprises a plurality of distinct lipids. In one embodiment, the nanoparticle comprises one or more of DPPC, distearoylphosphatidylcholine (DSPC), 1,2-dimyristoyl-sn-glycero-3-phosphoglycerol (DMPG), 1,2-dimyristoyl-sn-glycero-3-phosphocholine (DMPC), 1,2-dioleoyl-sn-glycero-3-phosphatidylcholine (DOPC), cholesterol or 1,2-distearoyl-sn-glycero-3-phosphoethabnolaminie (DSPE)-PEG, or any combination thereof. In one embodiment, the nanoparticle comprises a molar ratio of DPPC, DMPC, DOPC, or DSPC of 55 to 75. In one embodiment, the nanoparticle comprises a molar ratio of DPPC, DMPC, DOPC, or DSPC 60 to 70. In one embodiment, the nanoparticle comprises a molar ratio of DMPG of 10 to 30. In one embodiment, the nanoparticle comprises a molar ratio of DMPG of 15 to 25. In one embodiment, the nanoparticle comprises a molar ratio of cholesterol of 5 to 15. In one embodiment, the nanoparticle comprises a molar ratio of cholesterol of 7 to 12. In one embodiment, the nanoparticle comprises a molar ratio of DSPE-PEG of 1 to 15. In one embodiment, the nanoparticle comprises a molar ratio of DSPE-PEG of 2 to 10. In one embodiment, the PEG has a molecular weight of about 1K to 3K or 2K to 4K. In one embodiment, the PEG has a molecular weight of at least 2K and less than 10K or at leasty 0.3K to 5K. In one embodiment, the nanoparticle is a mesoporous silica nanoparticle. In one embodiment, the positively charged lipd bilayer comprises DPPC, DOTAP and cholesterol optionally at a molar ratio of about 80:10:10. In one embodiment, the anionic lipid p;ayer comprises DPPC, DMPG, cholesterol and DSPE-PEG2K optionally at a molar ration of 65:20:10:5. In one embodiment, the nanoparticle comprises one or more cargo molecules which are optionally negatively charged. In one embodiment, the cargo comprises one or more prophylactic, diagnostic or therapeutic agents. In one embodiment, the cargo comprises RNA. In one embodiment, the RNA comprises siRNA or mRNA, e.g., having one or more modified nucleotides. In one embodiment, the cargo is between the nanoparticle and the first coat or in the first coat. In one embodiment, the nanoparticle comprises one or more distinct targeting moieties. In one embodiment, the targeting moiety is specific for PCA3, EGFR, folate or CD19, and is optionally an antibody or an antigen binding fragment thereof. In one embodiment, the polyamine has a molecular weight of less than 20 KDa or a molecular weight of 25K or less. In one embodiment, the polyamine has a molecular weight of less than 10 KDa. In one embodiment, the polyamine has a molecular weight of less than 5 KDa. In one embodiment, the polyamine has a molecular weight of about 1 kDa to about 2 KDa. In one embodiment, the polyamine comprises linear or branched polyethylenimine (PEI), polyacrylamide (PAM), poly-lysine, spermine, spermidine, chitosan, or a diamine. In one embodiment, the nanoparticle of has a diameter of about 100 nm to 400 nm. In one embodiment, the nanoparticle comprises pores of less than 25 nm in diameter. In one embodiment, the pores have a diameter of about 1 to 10 nm. In one embodiment, the weight ratio of polyamine to nanoparticle is 0.05 to 10. In one embodiment, the weight ratio of polyamine to nanoparticle is 0.05 to 2 or 0.05 to 0.5. In one embodiment, the weight ratio of polyamine to nanoparticle is 0.1 to 0.6. In one embodiment, the nanoparticle of any one of claims 2 to 41 which comprises Gemcitabine and an anti-CD19 molecule, Doxorubicin and an anti-EGFR molecule, Afatinib and folate or a PCA3 siRNA and PCA3.

Further provided is a method of making the nanoparticle, comprising: combining an amount of nanoparticles and a composition comprising an amount of a polyamine or positively charged lipids so as to form a polyamine or positively charged lipid bilayer coat on the nanoparticles; and combining the polyamine or positively changed lipid coated nanoparticles and a composition comprising an amount of anionic lipids so as to form an anionic lipid coat on the polyamine or positively charged lipid bilayer coated nanoparticles. In one embodiment, the method further comprises combining one or more prophylactic, diagnostic or therapeutic agents with the nanoparticles and the polyamine or positively charged lipids. In one embodiment, the method further comprises combining one or more prophylactic, diagnostic or therapeutic agents with the nanoparticles before the nanoparticles are combined with the polyamine or positively charged lipids. In one embodiment, the method further comprises combining one or more prophylactic, diagnostic or therapeutic agents with the polyamine or positively charged lipid bilayer coated nanoparticles before the polyamine or positively charged lipid bilayer coated nanoparticles are combined with the anionic lipids. In one embodiment, at least one of the distinct anionic lipids comprises a targeting moiety.

Also provided is a method comprising administering to a mammal a composition comprising the nanoparticle. In one embodiment, the mammal is a human. In one embodiment, the mammal has cancer. In one embodiment, the nanoparticle comprises a therapeutic, diagnostic or prophylactic cargo. In one embodiment, the anionic lipid bilyaer of the nanoparticles comprises a targeting moiety. In one embodiment, targeting moiety is specific for PCA3, EGFR, folate or CD19. In one embodiment, the mammal has leukemia, prostate cancer, lung cancer or colorectal cancer. In one embodiment, the nanoparticle comprises capecitabine, gemcitabine, irinotecan, cyclophosphamide, cytarabine, vincristine, dexamethanose, or thioguanine.

In one embodiment, the cargo comprises a negatively charged molecule and the lipid bi-layer comprises a targeting molecule, for example, anti-PCA3, anti EGF, anti CD19 or anti-folate, e.g., a folate receptor. In one embodiment, the nanoparticle comprises Gemcitabine and binds CD19, Doxorubicin and binds EGFR, Afatinib and binds folate, or PCA3 siRNA and binds PCA3.

The invention will be described by the following non-limiting examples.

### Example 1

To drive the loading of negatively charged cargo, e.g., siRNA, into negatively charged MSNs via ‘fusion’, a soft positive layer is employed to create a silica/soft shell triplex.

Choice of Triplex components: Two approaches were evaluated. In both approaches, negatively charged MSNs were used. Then a positive layer was fused before or after siRNA addition. The siRNA loaded system then encapsulated within an optimized targetable negatively charged lipid bilayer.

Approach #1 MSN/L+/L″: The use of a positive lipid to create an intermediate layer coated by a targetable negatively charged lipid layer.

Approach #2 MSN/PEI2w/L: The use of a small polyamine to create an intermediate polymer-based layer coated by a targetable negatively charged lipid layer.

Approach #1 results in only 2-25% siRNA loading (1-10 μg/mg MSN) depending on the lipid/silica ratio and the fusion order (whether before or after adding siRNA). While using a standard lipid/MSN ratio=2-5, the loading extent was less than 10%. This loading capacity was attributed to the undesirable binding of negatively charged siRNA to excess positively charged liposomes that are not fused on the MSN surface and subsequently removed.

Reducing the lipid/MSN ratio (to 0.25 and 0.5) tends to increase the loading to (13-24%) but was accompanied with an increase (20-55%) of the size of the final product along with its polydispersity index. The low to moderate siRNA loading using a positive lipid layer led to the use of a polyamine as an intermediate layer. The loading and release of siRNA was evaluated by using a small water-soluble branched PEI (MW=2 KDa). In approach #2, we different PEI/MSN weight ratios (0.1-5) were evaluated where it was determined that a loading extent of >75% was obtained in all the studied cases. The balance of loading extent with lipid coated nanoparticle size corresponded to a PEI/MSN ratio of 0.4, where the loading of siRNA was >90% (corresponding to up to 50 μg siRNA/mg MSN) and the size polydispersity index remained stable. The zeta potential results of the stepwise fabrication of the Triplex confirms the successful conjugation steps. MSN morphology, including pore size and surface area were characterized. Specifically, as the loading of siRNA is driven by an electrostatic interaction with PEI, the role of the pore size was investigated. PEI was incubated with nanoparticles (NPs) of three increasing pore sizes (dense Stöber nanoparticles, MSNs-2 nm and MSNs-8 nm) and with different PEI/NP ratios. The thermogravimetric results show that non-porous Stober nanoparticles hold less than 2% PEI for ratios PEI/NP=0.4 and 5. On the other hand, both MSN-2 nm and MSN-8 nm show a high (ca. 20%) PEI content that increases to 28% with increasing PEI/MSN ratio. By calculating the % PEI normalized to the surface area of nanoparticles, we show that MSN-8 nm has the highest loading extent although possesses a lower surface area (650 m²/g) compared to MSN-2 nm (950 m²/g) which is indicative of the role of the pore size in PEI attachment and suggests that PEI follows the surface roughness.

### Example 2

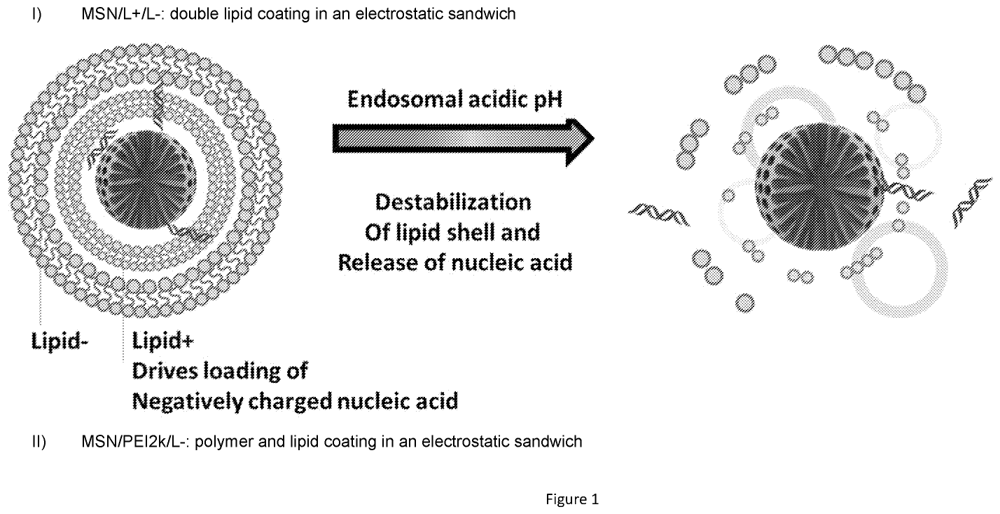

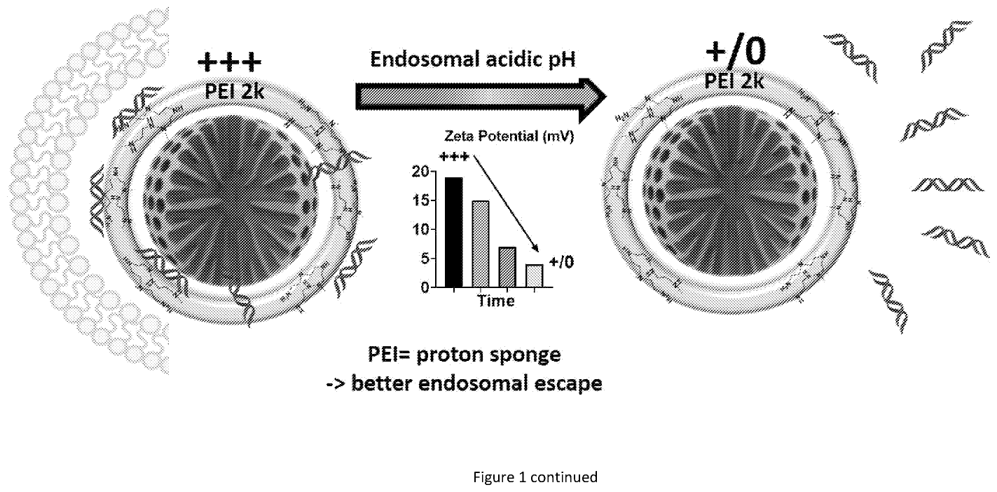

The loading of negatively charged RNA into negatively charged MSN was accomplished via ‘fusion’ of a soft positive layer to create a silica/soft shell triplex (see FIG. 1).

Choice of Triplex components: Two approaches were evaluated (FIG. 1). In both approaches, negatively charged MSNs were used. Then a positive layer was fused before or after siRNA addition. The siRNA loaded system then encapsulated within an optimized targetable negatively charged lipid bilayer.

Approach #1 MSN/L⁺/L: The use of a positive lipid to create an intermediate layer coated by a targetable negatively charged lipid layer.

Approach #2 MSN/PEI₂w/L: The use of a small polyamine to create an intermediate polymer-based layer coated by a targetable negatively charged lipid layer.

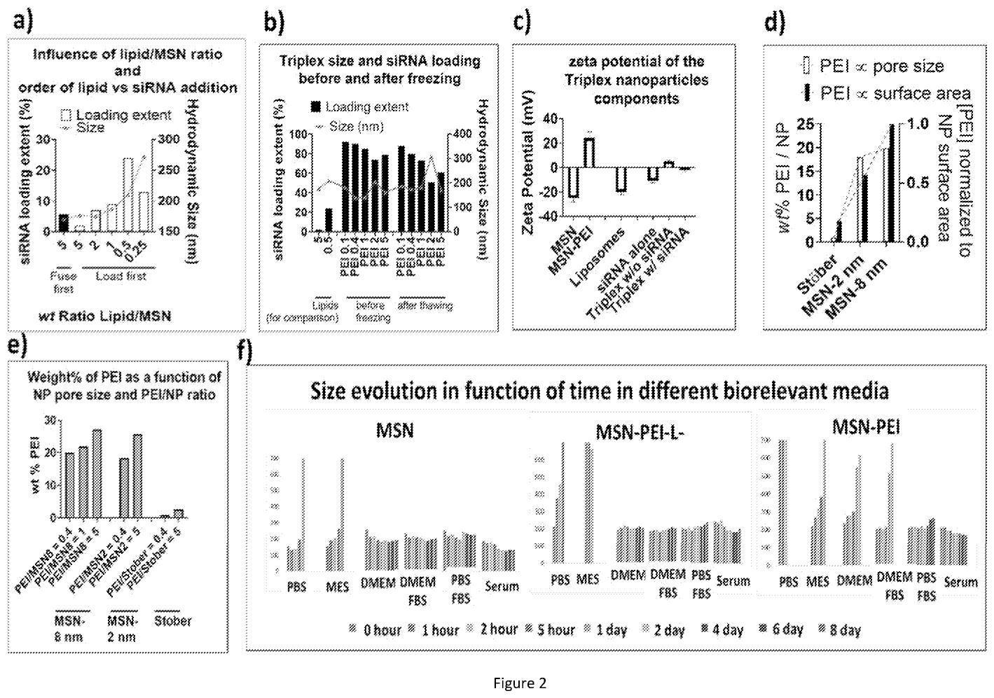

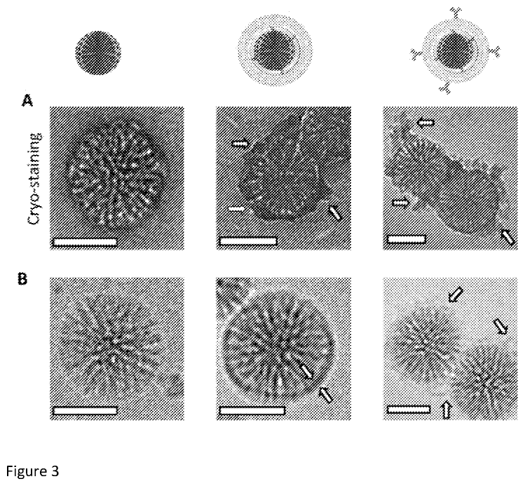

Approach #1 resulted in only 2-25% siRNA loading (1-10 μg/mg MSN) depending on the lipid/silica ratio and the fusion order (whether before or after adding SIRNA). While using a standard lipid/MSN ratio=2-5, the loading extent was less than 10%. This loading capacity was attributed to the undesirable binding of negatively charged siRNA to excess positively charged liposomes that are not fused on the MSN surface and subsequently removed. Reducing the lipid/MSN ratio (to 0.25 and 0.5) tends to increase the loading to (13-24%) but was accompanied with an increase (20-55%) of the size of the final product along with its polydispersity index (FIG. 2A). The low to moderate siRNA loading using a positive lipid layer led to a different approach that uses a polyamine as an intermediate layer (FIG. 2F). The loading and release of siRNA was evaluated by using a small water-soluble branched PEI (MW=2 KDa). In approach #2,different PEI/MSN weight ratios (0.1-5) were evaluated where it was determined that a loading extent of >75% was obtained in all the studied cases. The best balance of loading extent with lipid coated nanoparticle size corresponded to a PEI/MSN ratio of 0.4, where the loading of siRNA was >90% (corresponding to up to 50 μg siRNA/mg MSN) and the size polydispersity index remained stable (FIG. 2B). The zeta potential results of the stepwise fabrication of the Triplex are shown in FIG. 2C and confirmed the successful conjugation steps. MSN morphology, including pore size and surface area were evaluated. Specifically, as the loading of siRNA is driven by an electrostatic interaction with PEI, the role of the pore size was evaluated. Therefore, PEI was incubated with nanoparticles (NPs) of three increasing pore sizes (dense Stöber nanoparticles, MSNs-2 nm and MSNs-8 nm) and with different PEI/NP ratios. The thermogravimetric results (FIG. 2E) show that non-porous Stöber nanoparticles hold less than 2% PEI for ratios PEI/NP=0.4 and 5. On the other hand, both MSN-2 nm and MSN-8 nm show a high (ca. 20%) PEI content that increases to 28% with increasing PEI/MSN ratio. By calculating the % PEI normalized to the surface area of nanoparticles (FIG. 2D), MSN-8 nm was found to have the highest loading extent although possesses a lower surface area (650 m²/g) compared to MSN-2 nm (950 m²/g) which is indicative of the role of the pore size in PEI attachment and suggests that PEI follows the surface roughness.

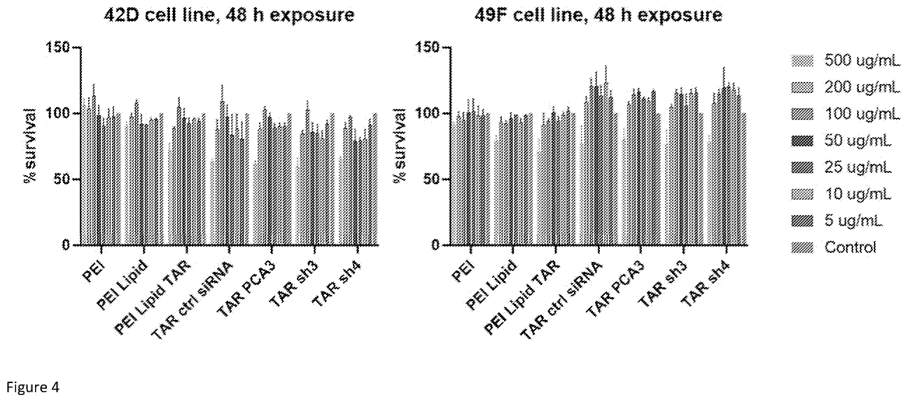

FIG. 4 shows that at an exemplary useful range of particles 10-200 μg/mL negligible cell death occurs indicating that the system is useful for targeting the cells without killing them. Control=particles no targeting agent incorporated (no anti PCA3 conjugated). PCA3=prostate cancer antigen 3 (characteristic and overxpressed on prostate cancer cells). TAR=Targeting. Means particles were conjugated with anti PCA3 targeting agent (minibody). The PCA3 peptide sequence is: 5FAM-C6-Lys (Biotin or azide or nothing)-Gly-Gly-Gly-Cys-Ser-Asn-Thr-Arg-Val-Ala-Pro-Cys-(Cys&Cys Bridge; SEQ ID NO: 34)=5FAM-C6-K (Biotin or azide or nothing)-GGCSNTRVAPC-(Cys&Cys Bridge; SEQ ID NO: 34). Biotin or azide or nothing=the chemistry of the PCA3 antibody to make attach to nanoparticles

Sh3:
(SEQ ID NO: 30)
GACCUUUCUACACUGUAGAAUAACA
(sense),
(SEQ ID NO: 31)
UGUUAUUCUACAGUGUAGAAAGGUC
(antisense)
Sh4:
(SEQ ID NO: 32)
GACCCUUCGUGUUGCUGCCUAAUAU
(sense)
(SEQ ID NO: 33)
AUAUUAGGCAGCAACACGAAGGGUC
(antisense)

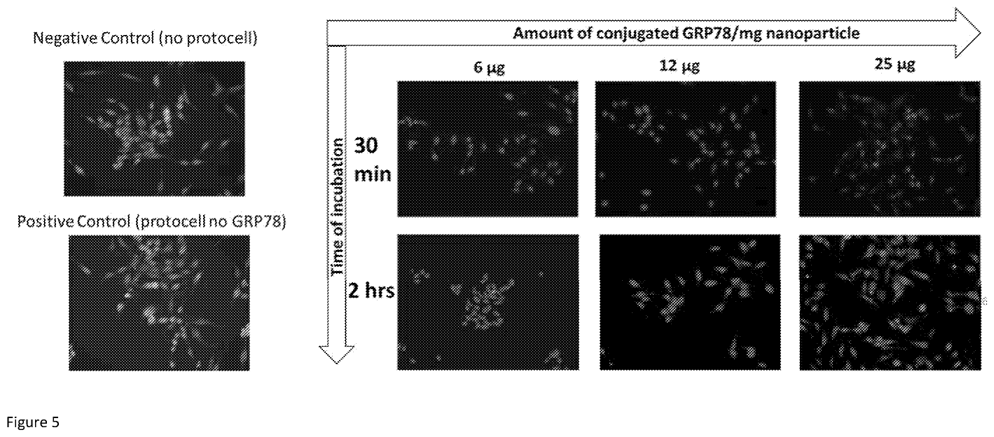

FIG. 5 shows that a targeting agent (GRP78) that targets prostate cancer cell line is conjugated at different ratio to the nanoparticle surface. The more targeting agent, the quicker and more pronounced is the uptake. Lipid coated nanoparticle=lipid-coated nanoparticle.

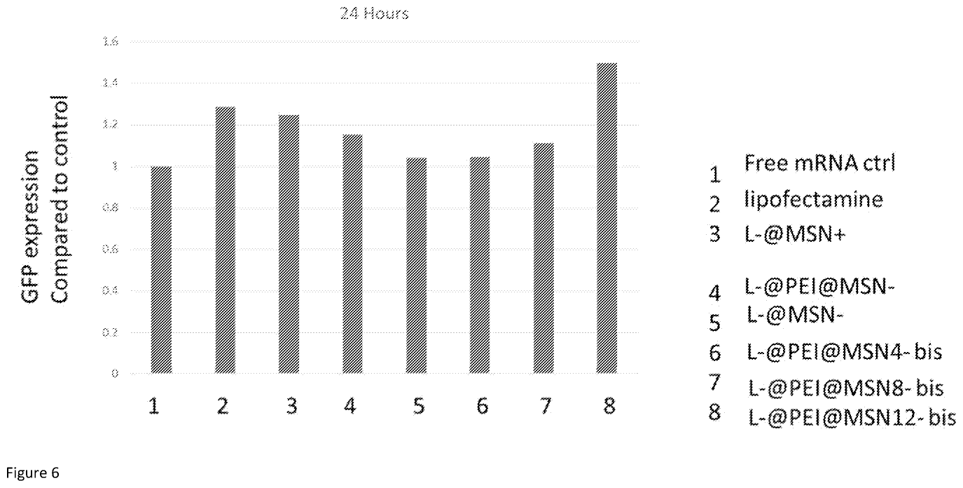

The delivery of mRNA may be accomplished using the lipid nanoparticles. FIG. 6 shows that some particle strategies increase the protein expression more than the gold standard lipofectamine messenger max. Specifically, while lipofectamine increases by 1.3x fold the GFP expression, the samples 8 reached 1.5x fold so at least 15% better than the gold standard used for transfection (https://www.thermofisher.com/order/catalog/product/LMRNA001 #: ˜: text=Lipofectamine % E2%84% A2%20MessengerMAX % E2%84% A2%20reagent %20 is %20designed %20 to % 20transfect %20a, to %20 other %20lipid %20based %20reagents.)

Other samples having aminated MSN with L—may also be employed, e.g., the amination is the positively charged layer.

Examples of nanoparticles are those having a positive lipid layer, e.g., DPPC/DOTAP/chol: 80/10/10 (molar ratio), optionally in the absence of PEG, and a negative lipid layer, e.g., DPPC/DMPG/cholesterol/DSPE-PEG2K: 65:20:10:5 (molar ratio), and those having a non-lipid polycation layer and a negative lipid layer (e.g., DPPC/DMPG/cholesterol/DSPE-PEG2K: 65:20:10:5 (molar ratio).

### Example 3

Exemplary nucleic acid cargoes are listed in Tables 1-3.

TABLE 1
Exemplary Antisense Olgonucleotides (ASO)

| Study id; year of | | |
|---|---|---|
| registration; country | | Target mRNA for |
| of conduct of study | Disease condition | ASO |
| NCT02627820; 2015; | Senile cardiac amyloidosis | Transthyretin |
| United States | | |
| NCT02406833; 2015; | Glaucoma patients undergoing | Transforming growth |
| Germany | trabeculectomy | factor-β. |
| NCT00780052; 2008; | Advanced haematological | Phase 1; c-myb ASO |
| United States of | malignancies (acute myeloid or | |
| America | lymphoid leukaemia; or | |
| | myeloproliferative disorder (MPD) | |
| | including chronic myelogenous | |
| | leukaemia (CML); or | |
| | myelodysplastic syndrome (MDS); | |
| | or non-Hodgkin's lymphoma | |
| | (including CLL); or multiple | |
| | myeloma) | |
| NCT00159250; 2005; | Duchenne muscular dystrophy | Phase 1/2 studies |
| United Kingdom | | against AVI-4658 |
| | | PMO |
| NCT02417753; 2015; | Malignant ascites | Phase 2; STAT3 |
| United States of | | |
| America | | |
| NCT02507583; 2015; | Malignant glioma | Phase 1; Insulin like |
| United States of | | growth factor-1 |
| America | | |
| NCT00466583; 2007; | Advanced solid tumours or | Phase 1; |
| United States of | lymphoma | Hypoxia-inducible |
| America | | factor-1α |
| NCT01550523; 2012; | Recurrent malignant glioma | Phase 1; Insulin like |
| United States of | | growth factor receptor |
| America | | |
| NCT00100672; 2005; | Advanced cancer | Phase 1; c-raf |
| United States of | | |
| America | | |
| NCT01120288; 2010; | Advanced solid tumours with | Phase 1; Hypoxia |
| United States of | metastasis | inducible factor-1α |
| America | | |
| NCT01159028; 2010; | Recurrent adult acute myeloid | Phase 1; Growth |
| United States of | leukaemia or acute lymphoblastic | factor receptor bound |
| America | leukaemia or myelodysplastic | protein-2 |
| | syndrome or Ph1 Positive chronic | |
| | myeloid leukaemia | |
| NCT00959868; 2009; | Superficial bladder tumour | Phase 1; Heat shock |
| Canada | | protein 27 |
| NCT00048321; 2002; | Rheumatoid arthritis | Phase 2; Tumor |
| United States of | | necrosis factor |
| America | | |
| NCT02423590; 2014; | Advanced squamous cell lung | Phase 2; Heat shock |
| United Kingdom | cancer | protein 27 |
| NCT00445913; 2007; | Type 1 diabetes mellitus | Phase 1; CD40, CD80 |
| United States of | | and CD86 |
| America | | |
| NCT01470911; 2011; | Bronchial asthma | Phase 1; GATA-3 |
| Germany | | |
| NCT01554319; 2012; | | |
| Germany | | |
| NCT01577953; 2012; | | |
| Germany | | |
| NCT01743768; 2012; | Bronchial asthma | Phase 2; GATA-3 |
| Germany | | |
| NCT02564354; 2015; | Cystic fibrosis | Phase 1; ΔF508 |
| United States of | | |
| America | | |
| NCT02532764; 2015; | Cystic fibrosis | Phase 1/2; ΔF508 |
| United States of | | |
| America | | |
| NCT00021749; | Chronic lymphocytic leukaemia | Phase 1/2; Bcl-2 |
| United States of | | |
| America | | |
| NCT00002592; 1999; | Chronic myelogenous leukaemia | Phase 2; c-myb |
| United States of | | |
| America | | |
| NCT00487786; 2007; | Prostate cancer, ovarian cancer, | Phase 1; Heat shock |
| United States of | non-small cell lung cancer, breast | protein 27 |
| America | cancer, bladder cancer | |
| NCT00005032; 2000; | Recurrent small cell lung cancer | Phase 1/2; Bcl-2 |
| United States of | | |
| America | | |
| NCT00059813; 2003; | Metastatic renal cell cancer | Phase 2; Bcl-2 |
| United States of | | |
| America | | |
| NCT00078234; 2004; | Chronic lymphocytic leukaemia | Phase 1/2; Bcl-2 |
| United States of | | |
| America | | |
| NCT00017251; 2001; | Extensive-Stage Small Cell Lung | Phase 1; Bcl-2 |
| United States of | Cancer | |
| America | | |
| NCT00471432: 2007; | Bladder cancer, breast cancer, | Phase 1; Clusterin |
| United States of | kidney cancer, lung cancer, ovarian | |
| America | cancer, prostate cancer | |
| NCT00063934; 2003; | Breast cancer | Phase 1/2; Bcl-2 |
| United States of | | |
| America | | |
| NCT01563302; 2012; | Lymphoma | Phase 1/2; STAT 3 |
| United States of | | |
| America | | |
| NCT00016263; 2001; | Malignant melanoma | Phase 3; Bcl-2 |
| United States of | | |
| America | | |
| NCT00258375; 2005; | Metastatic breast cancer | Phase 2; Clusterin |
| United States of | | |
| America | | |
| NCT00070083; 2003; | Diffuse large B cell lymphoma | Phase 1; Bcl-2 |
| United Kingdom | | |
| NCT00017602; 2001; | Multiple myeloma | Phase 3; Bcl-2 |
| United States of | | |
| America | | |
| NCT00365781; 2006; | Type 2 diabetes mellitus | Phase 1; Tyrosine |
| United States of | | phosphatase 1B |
| America | | |
| NCT00030641; 2002; | Non small cell lung cancer | Phase 2/3; Bcl-2 |
| United States of | | |
| America | | |
| NCT00070343; 2003; | Malignant melanoma | Bcl-2 |
| United States of | | |
| America | | |
| NCT00024440; 2001; | Chronic lymphocytic leukaemia | Phase 3; Bcl-2 |
| United States of | | |
| America | | |
| NCT00085228; 2004; | Adenocarcinoma prostate | Phase 2; Bcl-2 |
| Belgium | | |
| NCT00543205; 2007; | Melanoma | Phase 2/3; Bcl-2 |
| United States of | | |
| America | | |
| NCT00054106; 2003; | Prostate cancer | Phase 1; Bcl-2 |
| United States of | | |
| America | | |
| NCT01710852; 2012; | Paroxysmal atrial fibrillation | Phase 2; CRP |
| United States of | | |
| America | | |
| NCT00004870; 2000; | Colorectal cancer | Phase 1/2; Bcl-2 |
| United States of | | |
| America | | |
| NCT00003236; 1999; | Metastatic breast cancer | Phase 2; Protein |
| United States of | | kinase-alpha |
| America | | |
| NCT01120470; 2010; | Castration resistant prostate cancer | Phase 2; Heat shock |
| United Kingdom | | protein-27 |
| NCT00543231; 2007; | Solid tumours | Phase 1; Bcl-2 |
| United States of | | |
| America | | |
| NCT00636545 ; | Solid tumours | Phase 1; Bcl-2 |
| 2007; United States | | |
| of America | | |
| NCT00896857; 2009; | Breast cancer | Bcl-2 |
| United States of | | |
| America | | |
| NCT00024648; 2001; | Advanced malignancies | Phase 1; Raf-1 |
| United States of | | |
| America | | |
| NCT00024661; 2001; | Advanced solid tumours | Phase 1; Raf-1 |
| United States of | | |
| America | | |
| NCT02079688; 2014; | Atopic eczema | Phase 2; GATA-3 |
| United States of | | |
| America | | |
| NCT01839604; 2013; | Metastatic hepatocellular carcinoma | Phase 1; STAT-3 |
| Hong Kong | | |
| NCT00264966; 2005; | Asthma | Phase 1/2; CCR-3 |
| Canada | | |
| NCT00056173; 2003; | Renal cell carcinoma | Phase 1/2; |
| United States of | | ribonucleotide |
| America | | reductase |
| NCT00048113; 2002; | Crohn's disease | Phase 3; intercellular |
| United States of | | adhesion molecule |
| America | | |
| NCT00048295; 2002; | Crohn's disease | Phase 3; intercellular |
| United States of | | adhesion molecule |
| America | | |
| NCT01780545; 2013; | Metastatic bladder cancer | Phase 2; Heat shock |
| United States of | | protein 27 |
| America | | |
| NCT01598948; 2012; | Atherosclerosis | Phase 3; |
| Germany | | apolipoprotein B |
| NCT01083615; 2010; | Prostate cancer | Phase 3; Clusterin |
| United States of | | |
| America | | |
| NCT02549651; 2015; | Diffuse large B-cell lymphoma | Phase 1; STAT 3 |
| United States of | | |
| America | | |
| NCT02243124; 2014; | Myelodysplastic syndrome | Phase 1; P 53 |
| United States of | | |
| America | | |
| NCT02144051; 2014; | Advanced solid tumours with | Phase 1; Androgen |
| United States of | androgen receptor pathway as a | receptor |
| America | potential factor | |
| NCT00363974; 2006; | Acute myelomonocytic leukaemia | Phase 1; X-linked |
| United States of | | inhibitor of apoptosis |
| America | | protein |
| NCT00054548; 2003; | Advanced solid tumours | Phase 1; Bcl-2 |
| United States of | | |
| America | | |
| NCT00557596; 2007; | Advance pancreatic cancer | Phase 1/2; X-linked |
| United States of | | inhibitor of apoptosis |
| America | | protein |
| NCT00558922; 2007; | Non-small cell lung cancer | Phase 1/2; X-linked |
| United States of | | inhibitor of apoptosis |
| America | | protein |
| NCT00080847; 2004; | Advanced diffuse large B-cell | Phase 2; Bcl-2 |
| United States of | non-Hodgkin's lymphoma | |
| America | | |
| NCT00385775; 2006; | Advanced tumours | Phase 1; X-linked |
| United States of | | inhibitor of apoptosis |
| America | | protein |
| NCT00967512; 2009; | Acute myelogenous leukaemia | Phase 2; P 53 |
| United States of | | |
| America | | |
| NCT00558545; 2007; | Advanced breast cancer | Phase 1/2; X-linked |
| United States of | | inhibitor of apoptosis |
| America | | protein |

TABLE 2
siRNA

| Study id; year of | | |
|---|---|---|
| registration; | | |
| country of conduct | Disease | Phase of clinical trial; target |
| of study | condition | mRNA for ASO |
| NCT00716014; 2008; | Pachyonychia | Phase 1; PC keratins - K6a |
| United States of | congenital (PC) | |
| America | | |
| NCT00363714; 2006; | Age-related macular | Phase 1/2; Vascular endothelial |
| United States of | degeneration | growth factor |
| America | | |
| NCT00672542; 2008; | Metastatic melanoma | Phase 1; immunoproteasome beta |
| United States of | | subunits LMP2, LMP7, and MECL1 |
| America | | |
| NCT02166255; 2014; | Melanoma, renal or | Phase 1; E3 ubiquitin ligase Cbl-b |
| United States of | pancreatic cancer | |
| America | | |
| NCT00395057; 2006; | Age-related macular | Phase 2; Vascular endothelial |
| United States of | degeneration | growth factor |
| America | | |
| NCT00938574; 2009; | Advanced solid | Phase 1; Protein kinase 3 |
| Germany | tumours | |
| NCT00689065; 2008; | Solid tumours | Phase 1; ribonucleotide reductase |
| United States of | | |
| America | | |
| NCT01188785; 2010; | Pancreatic | Phase 1; KRAS (G12D) oncogene |
| United States of | adenocarcinoma | |
| America | | |
| NCT01591356; 2012; | Advanced cancers | Phase 1; EphA2 |
| United States of | | |
| America | | |
| NCT01064505; 2010; | Optic atrophy | Phase 1; pro-apoptotic protein, |
| United States of | | Caspase 2 |
| America | | |
| NCT00927459; 2009; | Hypercholesterolemia | Phase 1; polo-like kinase I, kinesin |
| United States of | | spindle protein |
| America | | |
| NCT01437007; 2011; | Hepatocellular | Phase 1; polo-like kinase 1 |
| United States of | carcinoma | |
| America | | |
| NCT01858935; 2013; | Healthy individuals | Phase 1; Heat shock protein 47 |
| United States of | | |
| America | | |
| NCT02314052; 2014; | Hepatocellular | Phase 1/2; MYC oncoprotein |
| United States of | carcinoma | |
| America | | |
| NCT02227459; 2014; | Hepatic fibrosis | Phase 1; Heat shock proteins |
| United States of | | |
| America | | |
| NCT02110563; 2014; | Multiple Myeloma | Phase 1; MYC oncoprotein |
| United States of | Non-Hodgkins | |
| America | Lymphoma | |
| NCT00306904; 2006; | Diabetic macular | Phase 2; Vascular endothelial |
| United States of | oedema | growth factor |
| America | | |
| NCT02250612; 2014; | Open angle | Phase 1; Beta-2 adrenergic |
| United States of | glaucoma | receptor |
| America | | |
| NCT01776658; 2012; | Dry eye syndrome | Phase 1/2; Vascular endothelial |
| United States of | | growth factor |
| America | | |
| NCT01739244; 2012; | Open angle | Phase 2; Vascular endothelial |
| United States of | glaucoma | growth factor |
| America | | |
| NCT01676259; 2012; | Advanced pancreatic | Phase 2; KRAS (G12D) oncogene |
| Israel | cancer | |
| NCT01438281; 2011; | Dry eye syndrome | Phase 1; transient receptor |
| Spain | | potential cation channel subfamily |
| | | V member 1 (TRPV1) |
| NCT00557791; 2007; | Age related macular | Phase 3; Vascular endothelial |
| United States of | degeneration | growth factor |
| America | | |
| NCT00802347; 2008; | Renal transplantation | Phase 1/2; P 53 |
| Canada | | |
| NCT01808638; 2013; | Pancreatic ductal | Phase 1/2; Protein kinase 3 |
| Germany | carcinoma | |
| NCT02596347; 2015; | Chronic beryllium | JAM2 |
| United States of | disease | |
| America | | |
| NCT00154934; 2005; | Pre-eclampsia | IL-10 |
| Taiwan | | |
| NCT01075360; 2010; | Neuroblastoma | E2F1 |
| Taiwan | | |
| NCT01834105; 2013; | Post menopausal | Phase 2; cardiotrophin-like |
| China | osteoporosis | cytokine factor 1 |
| NCT01227291; 2010; | Glaucoma | Phase 1/2; Beta-2 adrenergic |
| Spain | | receptor |
| NCT01382511; 2011; | Pachyonychia | STAT 1 |
| Israel | congenita | |
| NCT02528682; 2015; | Hematological | Phase 1/2; PD-1/PD-L1 |
| Netherlands | malignancies | co-inhibitory pathway |
| NCT02055846; 2012; | Prostate cancer | Heat shock protein 27 |
| France | | |
| NCT00554359; 2007; | Acute renal failure | Phase 1; P53 |
| United States of | | |
| America | | |
| NCT01058798; 2010; | Neuroblastoma | β1,4-N-acetylgalactosaminyltransferase |
| Taiwan | | III |
| NCT01445899; 2011; | Diabetic macular | Phase 2; hypoxia-inductible gene |
| Israel | oedema | |
| NCT00259753; 2005; | Age related macular | Phase 2; Vascular endothelial |
| United States of | degeneration | growth factor |
| America | | |

TABLE 3
Antisense oligonucleotides

| | Type of | | Route of | | Condition or | Clinical |
|---|---|---|---|---|---|---|
| Candidate | RNA | Vehicle | administration | Biological target | disease | trial number |
| QR-010 | ASO | NA | Inhalation | Cystic fibrosis | Cystic fibrosis | NCT02532764 |
| | | | | transmembrane | | |
| | | | | conductance | | |
| | | | | regulator (ΔF508 | | |
| | | | | mutation) | | |
| QR-421a | ASO | NA | Intravitreal | Usherin (exon | Retinitis | NCT03780257 |
| | | | injection | 13 of USH2A) | pigmentosa | |
| QR-110 | ASO | NA | Intravitreal | Centrosomal | Leber's | NCT03913143 |
| | | | injection | protein 290 | Congenital | |
| | | | | (p.Cys998X | amaurosis | |
| | | | | mutation) | | |
| QR-1123 | ASO | NA | Intravitreal | Mutant P23H of | Retinitis | NCT04123626 |
| | | | injection | the rhodopsin gene | pigmentosa | |

siRNA and miRNA

| | Type of | | Route of | | | |
|---|---|---|---|---|---|---|
| Candidate | RNA | Vehicle | administration | Biological target | Condition or disease | CT identifier |
| TD101 | siRNA | NA | Intralesional | Keratin 6A | Pachyonychia congenita | NCT00716014 |
| | | | injection | | | |
| PF-04523655 | siRNA | NA | Intravitreal | RTP801 | Age-related macular | NCT00713518 |
| | | | injection | | degeneration | |
| | | | | | Choroidal | NCT01445899 |
| | | | | | neovascularization | |
| | | | | | Diabetic retinopathy | |
| | | | | | Diabetic macular edema | |
| ALN-RSV01 | siRNA | NA | Inhalation of | Nucleocapsid protein of | Lung transplant patients | NCT01065935 |
| | | | nebulized | respiratory syncytial virus | infected with respiratory | |
| | | | solution | | syncytial virus | |
| ALN-VSP02 | siRNA | siRNA-LNP^{b} | i.v. infusion | Kinesin spindle protein and | Solid tumor | NCT01158079 |
| | | (2 siRNAs) | | vascular endothelial growth | | NCT00882180 |
| | | | | factor | | |
| TKM-080301 | siRNA | Stable nucleic | i.v. infusion | Polo-like kinase 1 | Neuroendocrine tumors | NCT01262235 |
| | | acid-lipid | | | | |
| | | particles | | | | |
| | | (SNALPs) | | | | |
| | | | | | Adrenocortical | |
| | | | | | carcinoma | |
| | | | | | Primary or secondary | NCT01437007 |
| | | | | | liver cancer | |
| | | | | | Advanced | NCT02191878 |
| | | | | | hepatocellular | |
| | | | | | carcinoma | |
| QPI-1007 | siRNA | NA | Intravitreal | Caspase 2 | Nonarteritic anterior | NCT02341560 |
| | | | injection | | ischemic optic | |
| | | | | | neuropathy | |
| | | | | | Acute primary angle | NCT01965106 |
| | | | | | closure glaucoma | |
| ALN-PCS02 | siRNA | siRNA-LNP | i.v. infusion | Proprotein convertase | Elevated | NCT01437059 |
| | | | | subtilisin/kexin type 9 | LDL-cholesterol | |
| siG12D- | siRNA | Miniature | Intratumoral | KRAS | Pancreatic cancer | NCT01676259 |
| LODER | | biodegradable | using | | | |
| | | polymeric matrix | endoscopic | | | |
| | | | ultrasound | | | |
| | | | biopsy needle | | | |
| DOPC- | siRNA | Neutral liposome | i.v. infusion | EphA2 | Advanced malignant | NCT01591356 |
| encapsulated | | | | | solid neoplasm | |
| siRNA | | | | | | |
| Atu027 | siRNA | Liposomal siRNA | i.v. infusion | Protein kinase N3 | Pancreatic cancer | NCT01808638 |
| SYL040012 | siRNA | NA | Ophthalmic | β2-Adrenergic receptor | Open angle glaucoma | NCT02250612 |
| | | | administration | | Ocular hypertension | |
| ND-L02-s0201 | siRNA | LNP | i.v. infusion | Heat shock protein 47 | Hepatic fibrosis | NCT02227459 |
| | | | | | Idiopathic pulmonary | NCT03538301 |
| | | | | | fibrosis | |
| QPI-1002 | siRNA | NA | i.v. injection | P53 | Delayed graft function | NCT02610296 |
| | | | | | Risk for AKI following | NCT03510897 |
| | | | | | cardiac surgery | |
| ALN-PCSSC | siRNA | GalNAc-siRNA | s.c. injection | Proprotein convertase | Elevated | NCT02314442 |
| (Inclisiran) | | | | subtilisin/kexin type 9 | LDL-cholesterol | |
| | | | | | Atherosclerotic | NCT02597127 |
| | | | | | cardiovascular disease | |
| | | | | | (or risk equivalents) | |
| | | | | | Renal impairment | NCT03159416 |
| | | | | | High cardiovascular | NCT03060577 |
| | | | | | risk and elevated | |
| | | | | | LDL-cholesterol | |
| | | | | | Atherosclerotic | NCT03399370 |
| | | | | | cardiovascular disease | NCT03400800 |
| | | | | | Elevated | |
| | | | | | LDL-cholesterol | |
| | | | | | Homozygous Familial | NCT03851705 |
| | | | | | Hypercholesterolemia | |
| | | | | | Atherosclerotic | NCT03814187 |
| | | | | | cardiovascular disease | |
| | | | | | (or risk equivalents) | |
| | | | | | Hypercholesterolemia | |
| | | | | | Elevated | |
| | | | | | LDL cholesterol | |
| SYL1001 | siRNA | NA | Ophthalmic | Transient receptor potential | Dry eye disease | NCT03108664 |
| | | | administration | cation channel subfamily V | | |
| | | | | Member 1 | | |
| ALN-GO1 | siRNA | GalNAc-siRNA | s.c. injection | hydroxyacid oxidase 1 | Primary hyperoxaluria | NCT04152200 |
| (Lumasiran) | | | | | type 1 | NCT03905694 |
| | | | | | | NCT03681184 |
| ALN-AT3 | siRNA | GalNAc-siRNA | s.c. injection | Antithrombin | Hemophilia | NCT03549871 |
| (Fitusiran) | | | | | | NCT03974113 |
| | | | | | | NCT03754790 |
| | | | | | | NCT03417102 |
| | | | | | | NCT03417245 |
| ALN-AAT02 | siRNA | GalNAc-siRNA | s.c. injection | Mutant of α-1 antitrypsin | ZZ Type α-1 Antitrypsin | NCT03767829 |
| | | | | | deficiency liver disease | |
| Patisiran (label | siRNA | LNP | i.v. infusion | transthyretin | hATTR with | NCT03997383 |
| expansion | | | | | cardiomyopathy hATTR | NCT03862807 |
| studies) | | | | | progression post liver | NCT02510261 |
| | | | | | transplant | NCT04201418 |
| | | | | | Polyneuropathy | (Phase IV) |
| ALN-CC5 | siRNA | GalNAc-siRNA | s.c. injection | Complement | IgA nephropathy Berger | NCT03841448 |
| (Cemdisiran) | | | | component C5 | disease | |
| ALN-AGT01 | siRNA | GalNAc-siRNA | s.c. injection | Angiotensinogen | Hypertension | NCT03934307 |
| ALN- | siRNA | GalNAc-siRNA | s.c. injection | Transthyretin | Amyloidosis, hereditary | NCT03759379 |
| TTRsc02 | | | | | transthyretin amyloidosis | |
| (Vutrisiran) | | | | | Transthyretin | NCT04153149 |
| | | | | | amyloidosis (ATTR) | |
| | | | | | with cardiomyopathy | |
| ARO-APOC3 | siRNA | TRiM ™ | s.c. injection | Apolipoprotein C3 | Hypertriglyceridemia | NCT03783377 |
| | | formulation | | | Familial | |
| | | | | | chylomicronemia | |
| ARO-ANG3 | siRNA | TRiM ™ | s.c. injection | Angiopoietin like protein 3 | Dyslipidemia | NCT03747224 |
| | | formulation | | | | |
| RG6346 | siRNA | GalNAc-siRNA | s.c. injection | Hepatitis B surface antigen | Chronic hepatitis B viral | NCT03772249 |
| (DCR-HBVS) | | | | | infection | |
| KrasG12D | siRNA | Mesenchymal | i.v. infusion | KRAS | Pancreatic cancer | NCT03608631 |
| siRNA | | Stromal cell | | | patients with | |
| (iExosomes) | | derived Exosomes | | | KrasG12D mutation | |
| OLX10010 | siRNA | cp-asiRNA | i.d./s.c. | Connective tissue growth | Hypertrophic scar | NCT03569267 |
| | | | injection | factor | | |
| ARO-AAT | siRNA | TRiM ™ | s.c. injection | Mutant of α1-antitrypsin | α1-Antitrypsin | NCT03946449 |
| | | formulation | | (Z-AAT) | deficiency | |
| ARO-HSD | siRNA | TRiM ™ | s.c. injection | Hydroxysteroid | Nonalcoholic | NCT04202354 |
| | | formulation | | 17β-dehydrogenase 13 | steatohepatitis | |
| ARO-HIF2 | siRNA | TRiM ™ | i.v. infusion | Hypoxia-inducible | Clear cell renal cell | NCT04169711 |
| | | formulation | | factor 2α | carcinoma | |
| DCR-PHXC | siRNA | GalXC-siRNA | s.c. injection | Lactate dehydrogenase A | Primary hyperoxaluria | NCT03847909 |
| | | | | | type 1 and type 2 | NCT04042402 |
| | | | | | | (Phase 3) |
| JNJ-3989 | siRNA | TRiM ™ | s.c. injection | Hepatitis B surface antigen | Chronic hepatitis B viral | NCT04208386 |
| | | formulation | | | infection | |
| | | | | | | NCT04439539 |
| BMT101 | siRNA | cp-asiRNA | i.d. injection | Connective tissue growth | Hypertrophic scar | NCT04012099 |
| | | | | factor | | |
| DCR-A1AT | siRNA | GalXC-siRNA | s.c. injection | α1- Antitrypsin | α1-Antitrypsin | NCT04174118 |
| | | | | | deficiency | |
| ARO-ENaC | siRNA | NA | Inhalation of | Epithelial sodium channel | Cystic fibrosis | NCT04375514 |
| | | | nebulized | α subunit | | |
| | | | solution | | | |
| AMG 890 | siRNA | GalNAc-siRNA | s.c. injection | Lipoprotein(a) | Elevated plasma | NCT04270760 |
| | | | | | lipoprotein(a) | |
| STP705 | siRNA | Histidine-lysine | Intralesional | TGF-β, Cox-2 | Cutaneous squamous | NCT04293679 |
| | | copolymer | injection | | cell carcinoma in situ | |
| | | peptide | | | | |
| Miravirsen | LNA- | NA | s.c. injection | miR-122 | Hepatitis C | NCT01200420 |
| | modified | | | | | NCT02508090 |
| | anti-miR | | | | | NCT02452814 |
| TargomiRs | miR-mimic | EDV (nonliving | i.v. infusion | miR-16 | Malignant pleural | NCT02369198 |
| | | bacterial | | | mesothelioma Non-small | |
| | | minicells) | | | cell lung cancer | |
| RG-125 | Anti-miR | GalNac | s.c. injection | miR-103/107 | Nonalcoholic | NCT02612662 |
| (AZD4076) | | | | | steatohepatitis | |
| SAR339375 | Anti-miR | NA | s.c. injection | miR-21 | Alport's syndrome | NCT02855268 |
| MRG-106 | LNA- | NA | i.v. infusion | miR-155 | Cutaneous T cell | NCT03713320 |
| (Cobomarsen) | modified | | | | lymphoma | |
| | anti-miR | | | | Mycosis fungoides | |
| | | | s.c. and | miR-155 | Lymphoma/leukemia | NCT02580552 |
| | | | intratumoral | | | |
| | | | injection | | | |
| | | | i.v. infusion | | | |
| MRG-110 | Anti-miR | NA | i.d. injection | miR-92a | Wound healing | NCT03603431 |
| MRG-201 | miR-mimic | Cholesterol | i.d. injection | miR-29 | Keloid | NCT03601052 |
| mRNA | | | | | | |
| Melanoma | mRNA | NA | i.d. injection | Melan-A Mage-A1 | Melanoma | NCT00204516 |
| mRNA | | | | Mage-A3 Survivin | | |
| vaccine | | | | GP100 Tyrosinase | | |
| Melanoma | mRNA | Protamine- | i.d. injection | Melan-A Mage-A1 | Melanoma | NCT00204607 |
| mRNA | | complexed | | Mage-A3 Survivin | | |
| vaccine | | mRNA | | GP100 Tyrosinase | | |
| CV9201 | mRNA | RNActive ® | i.d. injection | Tumor-associated antigens | Non-small cell | NCT00923312 |
| | | technology | | | lung cancer | |
| RBL001/ | mRNA | NA | i.nod. | Melanoma antigens | Melanoma | NCT01684241 |
| RBL002 | | | injection | | | |
| BNT114 | mRNA | Proprietary size- | i.v. infusion | Shared TAA for TNBC | TNBC | NCT02316457 |
| | | and charge-based | | and neoantigens identified | | |
| | | RNA-LPX | | by NGS | | |
| CV7201 | mRNA | RNActive ® | i.m. injection | Rabies virus glycoprotein | Rabies | NCT02241135 |
| | | technology | i.d. injection | | | |
| Lipo-MERIT | mRNA | Liposome- | i.v. infusion | RBL001.1 RBL002.2 | Melanoma | NCT02410733 |
| | | complexed | | RBL003.1 RBL004.1 | | |
| | | mRNA | | | | |
| mRNA-4157 | mRNA | Lipid | i.m. injection | Tumor-associated antigens | Solid tumors | NCT03313778 |
| | | encapsulated | | (personalized) | | |
| | | mRNA | | | | |
| | | | | | Melanoma | NCT03897881 |
| BI 1361849 | mRNA | RNActive ® | i.d. injection | Tumor-associated antigens | Non-small cell | NCT03164772 |
| | | technology | | | lung cancer | |
| mRNA-1647 | mRNA | Undisclosed | i.m. injection | Viral antigens | Cytomegalovirus | NCT03382405 |
| mRNA-1443 | | | | | infection | |
| mRNA1325 | mRNA | LNP | i.d. injection | Viral antigen | Zika virus infection | NCT03014089 |
| VAL506440 | mRNA | LNP | i.d. injection | H10N8 antigen influenza | Influenza | NCT03076385 |
| VAL339851 | mRNA | LNP | i.d. injection | H7N9 Antigen influenza | Influenza | NCT03345043 |
| mRNA-2416 | mRNA | LNP | Intratumoral | OX40 ligand | Advanced malignancies | NCT03323398 |
| | | | injection | | | |
| CV9202 | mRNA | RNActive ® | i.d. injection | Six non-small cell lung | Non-small cell | NCT03164772 |
| (BI 1361849) | | technology | | cancer antigens | lung cancer | |
| | | | | (NY-ESO-1, MAGE-C1, | | |
| | | | | MAGE-C2, survivin, | | |
| | | | | 5T4, and MUC-1) | | |
| Neo-antigen | mRNA | NA | s.c. injection | Tumor antigen | Digestive tract | NCT03468244 |
| mRNA | | | | | adenocarcinomas | |
| CV7202 | mRNA | LNP | i.m. injection | Rabies virus glycoprotein | Rabies | NCT03713086 |
| mRNA1653 | mRNA | LNP | i.d. injection | Viral antigen | Human | NCT03392389 |
| | | | | | metapneumovirus and | |
| | | | | | human parainfluenza | |
| | | | | | infection | |
| mRNA-2752 | mRNA | LNP | Intratumoral | OX40L IL-23 IL-36γ | Advanced malignancies | NCT03739931 |
| | | | injection | | | |
| mRNA-1944 | mRNA | LNP | i.v. infusion | Anti-Chikungunya | Chikungunya virus | NCT03829384 |
| | | | | virus mAb | infection | |
| BNT131 | mRNA | Proprietary size- | Intratumoral | IL-12sc IL-15sushi IFNα | Metastatic neoplasm | NCT03871348 |
| (SAR441000) | | and charge-based | injection | GM-CSF | | |
| | | RNA-LPX | | | | |
| mRNA-1893 | mRNA | LNP | i.m. injection | Undisclosed | Zika virus | NCT04064905 |
| mRNA-3704 | mRNA | LNP | i.v. infusion | Methylmalonyl-coenzyme | Methylmalonic acidemia | NCT03810690 |
| | | | | A mutase | | |
| mRNA-1273 | mRNA | LNP | i.m. injection | SARS-CoV-2 | COVID-19 | NCT04470427 |
| | | | | glycoproteins | | |
| BNT162 | mRNA | LNP | i.m. injection | Undisclosed SARS-CoV-2 | COVID-19 | NCT04380701 |
| | | | | proteins | | NCT04368728 |
| BNT122 | mRNA | Proprietary size- | i.v. infusion | Tumor-associated antigens | Colorectal cancer stage | NCT04486378 |
| (RO7198457) | | and charge-based | | | II/III Advanced | NCT03815058 |
| | | RNA-LPX | | | melanoma | |
| | | | | | Locally advanced or | NCT03289962 |
| | | | | | metastatic solid tumors | |
| CVnCoV | mRNA | LNP | i.m. injection | SARS-CoV-2 spike protein | COVID-19 | NCT04449276 |
| RNA | | | | | | |
| Aptamers | | | | | | |
| NOX-E36 | Aptamer | L-RNA, | i.v. injection | Chemokine | Chronic inflammatory | NCT00976729 |
| | | PEGylated | ms.c. injection | (cysteine-cysteine motif) | diseases Type 2 diabetes | |
| | | | | ligand 2 | mellitus Systemic lupus | |
| | | | | | erythematosus | |
| | | | | | Renal impairment | NCT01372124 |
| | | | | | Type 2 diabetes mellitus | NCT01547897 |
| | | | | | albuminuria | |
| NOX-A12 | Aptamer | L-RNA, | i.v. injection | Chemokine (C-X-C motif) | Autologous stem cell | NCT00976378 |
| | | PEGylated | | ligand 12 | transplantation | |
| | | | | Stromal-derived factor 1 | Chronic lymphocytic | NCT01486797 |
| | | | | | leukemia | |
| | | | | | Multiple myeloma | NCT01521533 |
| | | | | | Colorectal cancer | NCT03168139 |
| | | | | | Pancreatic cancer | |
| | | | | | Glioblastoma | NCT04121455 |
| NOX-H94 | Aptamer | L-RNA, | Undisclosed | Hepcidin | Anemia of chronic | NCT01691040 |
| | | PEGylated | | | disease | |
| | | | | | Anemia End-stage renal | NCT02079896 |
| | | | | | disease | |
| Zimura | Aptamer | NA | Intravitreal | Complement component 5 | Age-related macular | NCT03362190 |
| (ARC1905) | | | injection | | degeneration | |
| | | | | | Stargardt disease 1 | NCT03364153 |
| saRNA | saRNA | SMARTICLES ® | i.v. infusion | CCAAT enhancer binding | Hepatocellular | NCT02716012 |
| MTL- | | liposomal | | protein | carcinoma | |
| CEBPA | | nanoparticle | | | Liver cancer | |
| Other | Noncoding | NA | Intratumoral | Activation of | Melanoma Squamous | NCT03291002 |
| CV8102 | ssRNA | | injection | TLR7/8/RIG-1 | cell carcinoma of the | |
| | | | | | skin or head/neck | |
| | | | | | Adenoid cystic | |
| | | | | | carcinoma | |

aAn advanced clinical stage for each RNA is listed.
^{b}Abbreviations: AKI, acute kidney injury; cp-asiRNA, proprietary cell-penetrating asymmetric interfering RNA by OliX Pharmaceuticals; GalXCL, proprietary siRNA formulation by Dicerna Pharmaceuticals; GM-CSF, granulocyte-macrophage colony-stimulating factor; i.d., intradermal; IFN, interferon; II, interleukin; i.m., intramuscular; i.nod., intranodal; i.v., intravenous; LNA, locked nucleic acid; LNP, lipid nanoparticle; NA, not applicable; NGS, next-generation sequencing; s.c., subcutaneous; TRiM ™, targeted RNAi molecule, platform for ligand-mediated targeted RNA delivery by Arrowhead Pharmaceuticals; TAA, tumor-associated antigen; TNBC, triple-negative breast cancer.

### REFERENCES

Adamson, Cancer J. Clin., 65:212 (2015).
Akbarzadeh et al., Nanoscale Res. Lett., 8:102 (2013).
Allen and Cullis, Science, 303:1818 (2004).
Asefa and Tao, Chem. Res. Toxicol., 25:2265 (2012).
Ashley etExample al. ACS Nano, 6:2174 (2012).
Ashley et al., Nat. Mater., 10:389 (2011).
Attwood et al., Int. J. Mol. Sci., 14:3514 (2013).
Bae, J. Controlled Release, 133:2 (2009).
Bartlett et al., Proc. Natl. Acad. Sci. U.S.A 2007, 104, 15549-15554.
Bayerl and Bloom, Biophys. J., 58:357 (1990).
Bayu et al., Microporous Mesoporous Mater., 120:447 (2009).
Bertrand et al. Adv. Drug Deliv. Rev., 66:2 (2014).
Blanco et al., Nat. Biotechnol., 33:941 (2015).
Breus et al., ACS Nano, 3:2573 (2009).
Brinker and Scherer, Sol-Gel Science: The Physics and Chemistry of Sol-Gel Processing. Academic press: 2013.
Buranda et al., Langmuir, 19:1654 (2003).
Butler et al., Lipid coated nanoparticles: Modular Mesoporous Silica Nanoparticle-Supported Lipid Bilayers for Drug Delivery. Small 2016.
çağdaş et al., S. Liposomes as Potential Drug Carrier Systems for Drug Delivery. INTECH: 2014.
Carrol et al., Langmuir, 25:13540 (2009).
Cauda et al., Nano Lett., 10:2484 (2010).
Chauhan et al., Nat. Nanotechnol., 7:383 (2012).
Chen et al., Chem. Mater., 25:4269 (2013).
Choi et al., Nat. Biotechnol., 25:1165 (2007).
Cremer and Boxer, J. Phys. Chem. B, 103:2554 (1999).
Crist et al., Integr. Biol., 5:66 (2013).
Davis et al., Nat Rev Drug Discov., 7:771 (2008).
de Sousa Cavalcante et al., Eur. J. Pharmacol., 741:8 (2014).
Dengler et al., J. Controlled Release, 168:209 (2013).
Deshpande et al., Nanomedicine (London, U. K.), 8:10.2217 (2013).
Dobrovolskaia and McNeil, J. Controlled Release, 172:456 (2013).
Draz et al., Theranostics, 4:872 (2014).
Egusquiaguirre et al., Clin. Transl. Oncol., 14:83 (2012).
Elsabahy and Wooley, Chem. Soc. Rev., 41:2545 (2012).
Epler et al., Adv. Healthcare Mater., 1:348 (2012).
Estephan et al., Langmuir, 26:16884 (2010).
Farokhzad and Langer, ACS Nano, 3:16. (2009).
Federico et al., Int. J. Nanomed., 7:5423 (2012).
Ferrari, Nat. Nanotechnol., 3:131 (2008).
Garcia-Manyes et al., Biophys. J., 89:1812 (2005).
Han et al., ACS Appl. Mater. Interfaces, 7:3342 (2015).
He et al., Small, 7:271 (2011).
Henriksen et al., (2006).
Hrkach et al., Sci. Transl. Med., 4: 128ra39 (2012).
Huang et al., ACS Nano, 10:648 (2016).
Huang et al., ACS Nano, 5:5390 (2011).
li et al., Mol. Cell. Biol., 15:5770 (1995).
Iwamoto, Biol. Pharm. Bull., 36:715 (2013).
Iyer et al., Adv. Drug Delivery Rev., 65:1784 (2013).
Johnson et al., Biophys. J., 59:289 (1991).
Johnson et al., Biophys. J., 83:3371 (2002).
Keller et al., Phys. Rev. Lett., 84:5443 (2000).
Kirk-Othmer, Encyclopedia of Chemical Technology, Vol. 20, 3rd Ed., J. Wiley, N.Y.
Kohli et al., J. Control. Release, 190:274 (2014).
Kresge et al., Nature, 359:710 (1992).
Krishnan and Rajasekaran, Clin. Pharmacol. Ther., 95:168 (2014).
LaCasse et al., Nucl. Acids Res., 23:1647 (1995).
Lammers et al., J. Controlled Release, 161:175 (2012).
Lee et al., Acc. Chem. Res., 44:893 (2011).
Lee et al., Chem. Soc. Rev., 41:2656 (2012).
Leong et al., Nat. Protoc., 5:1406 (2010).
Li et al., Chem. Soc. Rev., 41:2590 (2012).
Liao et al., Interfaces, 3:2607 (2011).
Lin and Haynes, J. Am. Chem. Soc., 132:4834 (2010).
Lin and Haynes, J. Am. Chem. Soc., 4834 (2010)
Lin et al., Chem. Commun., 47:532 (2011).
Lin et al., Chem. Mater., 17:4570 (2005).
Lin et al., J. Am. Chem. Soc., 133:20444 (2011).
Lin et al., J. Phys. Chem. Lett., 3:364 (2012).
Lis et al., Physical Chemistry Chemical Physics, 13:17555 (2011).
Liu et al., ACS Nano, 10:2702 (2016).
Liu et al., Chem. Comm. 5100 (2009).
Liu et al., J. Am. Chem. Soc., 131:1354 (2009a).
Liu et al., J. Am. Chem. Soc., 131:7567 (2009b).
Lu et al., Nature, 398:223 (1999).
Lynch et al. Nano Today, 3:40 (2008).
Mackowiak et al., Nano Lett., 13:2576 (2013).
Markman et al., Adv. Drug Delivery Rev., 65:1866 (2013).
Marsh, CRC Press, (2013).
Meng et al., ACS Nano, 5:4434 (2011).
Meng et al., ACS Nano, 9:3540 (2015).
Moon et al., Nat. Mater., 10:243 (2011).
Mornet et al., Nano Lett., 5:281 (2005).
Murat Cokol, Raj Nair & Burkhard Rost, at the website ubic.bioc.columbia.edu/papers/2000 nls/paper.html #tab2.
Nag and Awasthi, Pharmaceutics, 5:542 (2013).
Nandiyanto et al., Microporous Mesoporous Mater., 120:447 (2009).
Nollert et al., Biophys. J., 69:1447 (1995).
Padera et al., Nature, 427:695 (2004).
Pattni et al., Chem. Rev. Chemical Reviews, 115:10938 (2015).
Peer et al., Nat. Nanotechnol., 2:751 (2007).
Perry et al., Nano Lett., 12:5304 (2012).
Petros and DeSimone, Nat. Rev. Drug Discov., 9:615 (2010).
Pluedemann, Silane Coupling Agents, Plenum Press, N.Y. 1982.
Porotto et al., PloS one, 6: e16874 (2011).
Reviakine and Brisson, Langmuir, 16:1806 (2000).
Reynolds et al., Toxicol. Appl. Pharmacol., 262:1 (2012).
Richter and Brisson, Biophys. J., 88:3422 (2005).
Riese et al., Mol. Cell. Biol., 15, 5770-5776.
Roggers et al., Mol. Pharm., 9:2770 (2012).
Sackmann, FEBS Lett., 346:3:16 (1994).
Sackmann, Handbook of Biological Physics, 1:213 (1995).
Savarala et al., Langmuir, 26:12081 (2010).
Schulze et al., Biochimica et Biophysica Acta (BBA)-Molecular Cell Research, 1793:674 (2009).
Seantier and Kasemo, Langmuir, 25:5767 (2009).
Shen et al., Nano Lett., 14:923 (2014).
Shi et al., Acc. Chem. Res., 44:1123 (2011).
Steichen et al., Eur. J. Pharm. Sci., 48:416 (2013).
Sun et al., Angew. Chem., Int. Ed., 53:12320 (2014).
Tarn et al., Acc. Chem. Res., 46:792 (2013).
Torchilin, Nat. Rev. Drug Discovery, 4:145 (2005).
Townson et al., J. Am. Chem. Soc., 135:16030 (2013).
Vargas et al., Adv. Drug Deliv. Rev., 59:1162 (2007).
Vivero-Escoto et al., Small, 6:1952 (2010).
Wang et al., ACS Nano, 4:4371 (2010).
Wang et al., Biomaterials, 34:7662 (2013).
Wang et al., J. Colloid Interface Sci., 385:41 (2012).
Wang et al., RSC Adv., 2:11336 (2012).
Weis, TIBS, 23:185 (1998).
Weis, Trends Biochem. Sci., 23:235 (1998).
Williford et al., Annu. Rev. Biomed. Eng., 16:347 (2014).
Yeagle, CRC press: (2004).
Yu et al., ACS Nano, 5:5717 (2011).
Zarschler et al., Nanoscale, 6:6046 (2014).
Zhang et al., Adv. Funct. Mater., 24:2352 (2014).
Zhang et al., Biomaterials, 35:3650 (2014).
Zhang et al., J. Am. Chem. Soc., 134:15790 (2012).
Zhu et al., Biomacromolecules, 15:1845 (2014).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A multi-layer coated nanoparticle wherein the nanoparticle comprises a first coat comprising a positively charged polymer or a positively charged lipid bilayer and a second coat comprising an anionic lipid bilayer, wherein the multi-layer nanoparticle optionally comprises one or more distinct cargo molecules, one or more distinct targeting moieties, or any combination thereof.

2. The nanoparticle of claim 1, wherein the positively charged polymer comprises a polyamine.

3. (canceled)

4. The nanoparticle of claim 1, wherein the positively charged lipid bilayer comprises one or more of dipalmitylphosphatidylcholine (DPPC), dioleoyl-3-trimethylammonium propatre (DOTAP), 1, 2-dioleoyloxy-3-dimethylamino propane (DODMA), or cholesterol.

5. The nanoparticle of claim 4, which comprises a molar ratio of DPPC of 70 to 90 or 30 to 90.

6. (canceled)

7. The nanoparticle of claim 4 which comprises a molar ratio DOTAP or DODMA of 5 to 15 or 5 to 50.

8. (canceled)

9. The nanoparticle of claim 4 which comprises a molar ratio of cholesterol of 5 to 15 or 0 to 40.

10. (canceled)

11. (canceled)

12. The nanoparticle of claim 1, wherein the anionic lipid bilayer comprises a plurality of distinct lipids.

13. The nanoparticle of claim 12 which comprises one or more of DPPC, distearoylphosphatidylcholine (DSPC), 1,2-dimyristoyl-sn-glycero-3-phosphoglycerol (DMPG), 1,2-dimyristoyl-sn-glycero-3-phosphocholine (DMPC), 1,2-dioleoyl-sn-glycero-3-phosphatidylcholine (DOPC), cholesterol or 1,2-distearoyl-sn-glycero-3-phosphoethabnolaminie (DSPE)-PEG, or any combination thereof.

14. The nanoparticle of claim 13 which comprises a molar ratio of DPPC, DMPC, DOPC, or DSPC of 55 to 75.

15. (canceled)

16. The nanoparticle of claim 13 which comprises a molar ratio of DMPG of 10 to 30.

17. (canceled)

18. The nanoparticle of claim 13 which comprises a molar ratio of cholesterol of 5 to 15.

19. (canceled)

20. The nanoparticle of claim 13 which comprises a molar ratio of DSPE-PEG of 1 to 15.

21. (canceled)

22. (canceled)

23. (canceled)

24. (canceled)

25. The nanoparticle of claim 1, wherein the positively charged lipid lipd bilayer comprises DPPC, DOTAP and cholesterol optionally at a molar ratio of about 80:10:10.

26. The nanoparticle of claim 1, wherein the anionic lipid bilaver comprises DPPC, DMPG, cholesterol and DSPE-PEG2K optionally at a molar ration of 65:20:10:5.

27. The nanoparticle of claim 1 which comprises one or more cargo molecules which are optionally negatively charged.

28. The nanoparticle of claim 27 wherein the cargo comprises one or more prophylactic, diagnostic or therapeutic agents.

29. (canceled)

30. (canceled)

31. (canceled)

32. The nanoparticle of claim 1 which comprises one or more distinct targeting moieties.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method of making the nanoparticle of claim 1, comprising:
combining an amount of nanoparticles and a composition comprising an amount of a polyamine or positively charged lipids so as to form a polyamine or positively charged lipid bilayer coat on the nanoparticles; and
combining the polyamine or positively changed lipid coated nanoparticles and a composition comprising an amount of anionic lipids so as to form an anionic lipid coat on the polyamine or positively charged lipid bilayer coated nanoparticles.

47. (canceled)

48. (canceled)

49. (canceled)

50. (canceled)

51. A method comprising administering to a mammal a composition comprising the nanoparticle of claim 1.

52. (canceled)

53. The method of claim 51 wherein the mammal has cancer.

54. The method of claim 51, wherein the nanoparticle comprises a therapeutic, diagnostic or prophylactic cargo.

55. The method of claim 51, wherein the anionic lipid bilayer of the nanoparticles comprises a targeting moiety.

56. (canceled)

57. (canceled)

58. The method of claim 51, wherein the nanoparticle comprises capecitabine, gemcitabine, irinotecan, cyclophosphamide, cytarabine, vincristine, dexamethasone, or thioguanine.


SEQUENCE LISTING (34 sequences)

SEQ ID NO: 1 - protein, 8 aa, synthetic construct
RRRRRRRR

SEQ ID NO: 2 - protein, 25 aa, synthetic construct
GLFHAIAHFIHGGWHGLIHGWYGGC

SEQ ID NO: 3 - protein, 12 aa, synthetic construct
SFSIILTPILPL

SEQ ID NO: 4 - protein, 25 aa, synthetic construct
GLFHAIAHFIHGGWHGLIHGWYGGC

SEQ ID NO: 5 - protein, 8 aa, synthetic construct
RRRRRRRR

SEQ ID NO: 6 - protein, 18 aa, synthetic construct
SFSIILTPILPLEEEGGC

SEQ ID NO: 7 - protein, 7 aa, synthetic construct
ASVHFPP

SEQ ID NO: 8 - protein, 7 aa, synthetic construct
TATFWFQ

SEQ ID NO: 9 - protein, 7 aa, synthetic construct
TSPVALL

SEQ ID NO: 10 - protein, 7 aa, synthetic construct
IPLKVHP

SEQ ID NO: 11 - protein, 7 aa, synthetic construct
WPRLTNM

SEQ ID NO: 12 - protein, 25 aa, synthetic construct
GLFHAIAHFIHGGWHGLIHGWYGGC

SEQ ID NO: 13 - protein, 8 aa, synthetic construct
RRRRRRRR

SEQ ID NO: 14 - protein, 30 aa, synthetic construct
WEARLARALARALARHLARALARALRAGEA

SEQ ID NO: 15 - protein, 30 aa, synthetic construct
WEAKLAKALAKALAKHLAKALAKALKAGEA

SEQ ID NO: 16 - protein, 30 aa, synthetic construct
WEAALAEALAEALAEHLAEALAEALEALAA

SEQ ID NO: 17 - protein, 23 aa, synthetic construct
GLFEAIEGFIENGWEGMIDGWYG

SEQ ID NO: 18 - protein, 30 aa, synthetic construct; identical to SEQ ID NO: 14

SEQ ID NO: 19 - protein, 30 aa, synthetic construct; identical to SEQ ID NO: 15

SEQ ID NO: 20 - protein, 30 aa, synthetic construct; identical to SEQ ID NO: 16

SEQ ID NO: 21 - protein, 23 aa, synthetic construct
GLFEAIEGFIENGWEGMIDGWYG

SEQ ID NO: 22 - protein, 42 aa, synthetic construct
GNQSSNFGPMKGGNFGGRSSGPYGGGGQYFAKPRNQGGYGGC

SEQ ID NO: 23 - protein, 7 aa, synthetic construct
RRMKWKK

SEQ ID NO: 24 - protein, 7 aa, synthetic construct
PKKKRKV

SEQ ID NO: 25 - protein, 16 aa, synthetic construct
KRPAATKKAGQAKKKK

SEQ ID NO: 26-29 - skipped (4 entries)

SEQ ID NO: 30 - RNA, 25 nt, synthetic construct
  source 1..25: mol_type = other RNA
gacctttctacactgtagaataaca

SEQ ID NO: 31 - RNA, 25 nt, synthetic construct
  source 1..25: mol_type = other RNA
tgttattctacagtgtagaaaggtc

SEQ ID NO: 32 - RNA, 25 nt, synthetic construct
  source 1..25: mol_type = other RNA
gacccttcgtgttgctgcctaatat

SEQ ID NO: 33 - RNA, 25 nt, synthetic construct
  source 1..25: mol_type = other RNA
atattaggcagcaacacgaagggtc

SEQ ID NO: 34 - protein, 11 aa, synthetic construct
GGCSNTRVAPC